(19) United States
(12) Patent Application Publication
(10) Pub. No.: US 2026/0085362 A1
(43) Pub. Date: Mar. 26, 2026

(54) NEUROENDOCRINE CARCINOMA DETECTION AND TREATMENT

(71) Applicant: Case Western Reserve University, Cleveland, OH (US)

(72) Inventors: Minh Lam, Highland Heights, OH (US); Afshin Dowlati, Solon, OH (US); Hung-Ying Kao, Shaker Heights, OH (US); Masaru Miyagi, Cleveland, OH (US)

(21) Appl. No.: 19/335,755

(22) Filed: Sep. 22, 2025

Related U.S. Application Data
(60) Provisional application No. 63/696,935, filed on Sep. 20, 2024.

(51) Int. Cl. C12Q 1/6886; C12Q 1/6851; G01N 21/64; G01N 33/574; G01N 33/68

(52) U.S. Cl. CPC C12Q 1/6886; C12Q 1/6851; G01N 21/6458; G01N 33/5759; G01N 33/6893; C12Q 2600/106; C12Q 2600/112; C12Q 2600/118; G01N 2333/70503

(57) ABSTRACT

The present disclosure relates generally to the field of medical oncology and, in particular, to methods and kits for identifying and treating neuroendocrine carcinomas, such as small cell neuroendocrine (SCN) tumors. Certain aspects of the present disclosure can include methods of diagnosing a patient with a neuroendocrine carcinoma, methods of determining a survival prognosis in a patient diagnosed with a neuroendocrine carcinoma, compositions that effectively inhibits or decreases an expression level of a hepatocyte cell adhesion molecule 2 (HEPACAM2) biomarker in vivo or ex vivo, and methods of treating a patient having, or suspected of having, a neuroendocrine carcinoma.

### RELATED APPLICATIONS

This application claims the benefit of U.S. Provisional Patent Application Ser. No. 63/696,935 (filed Sep. 20, 2024), the entirety of which is hereby incorporated by reference for all purposes.

### GOVERNMENT SUPPORT

This invention was made with government support under W81XWH-17-1-0195 awarded by the Department of Defense. The government has certain rights in the invention.

### SEQUENCE LISTING

The instant application contains a Sequence Listing which has been submitted electronically in XML format and is hereby incorporated by reference in its entirety. Said XML copy, created Sep. 17, 2025, is named CWR-033840_US_ORD_SL.xml and is 49,233 bytes in size.

### TECHNICAL FIELD

The present disclosure relates generally to the field of medical oncology and, in particular, to methods and kits for identifying and treating neuroendocrine carcinomas, such as small cell neuroendocrine (SCN) tumors.

### BACKGROUND

Although small cell lung cancer (SCLC) represents only a subgroup (15%) of all lung cancers, its annual mortality rate surpasses breast or prostate cancer in the United States. Patients who present with metastatic disease, i.e., extensive-stage SCLC, represent the majority of patients at diagnosis (˜75%) and have a 5-year survival of <2%. Poor survival remains directly attributable to a lack of progress in the early detection and treatment of this disease; chemotherapy remains the backbone of SCLC treatment and has not changed over the last three decades. Compared to non-small cell lung cancer (NSCLC), the application of genomics in SCLC has been futile in identifying actionable gene mutations. Most targetable genes in NSCLC, such as EGFR, ALK, ROS, or BRAF, are rarely altered in SCLC, which partly explains the repeated failure of past attempts to find effective targeted therapies for this cancer. Even currently approved immunotherapies have had limited success in SCLC compared to NSCLC.

### SUMMARY

The present disclosure relates generally to the field of medical oncology and, in particular, to methods and kits for identifying and treating neuroendocrine carcinomas, such as small cell neuroendocrine (SCN) tumors.

One aspect of the present disclosure can include a method of diagnosing a patient with a neuroendocrine carcinoma. One step of the method can comprise assaying for an expression level of a hepatocyte cell adhesion molecule 2 (HEPACAM2) biomarker in a biological sample previously obtained from the patient. A detected expression level of the HEPACAM2 biomarker that is higher than a control level of a HEPACAM2 biomarker can be indicative of a neuroendocrine carcinoma in the patient.

Another aspect of the present disclosure can include a method of determining a survival prognosis in a patient diagnosed with a neuroendocrine carcinoma. The method can comprise the steps of: assaying an expression level of a hepatocyte cell adhesion molecule 2 (HEPACAM2) biomarker in a biological sample previously obtained from the patient; and calculating a survival prognosis, based on the assayed expression level of the HEPACAM2 biomarker, comprising an overall survival (OS) and/or a progression-free survival (PFS). A detected expression level of the HEPACAM2 biomarker that is higher than a control level of a HEPACAM2 biomarker can be indicative of a lesser OS and/or a lesser PFS as compared to a control patient.

Another aspect of the present disclosure can include a method of treating a patient having, or suspected of having, a neuroendocrine carcinoma. The method can include administering a therapeutically effective amount of a therapeutic regimen to the patient. The neuroendocrine carcinoma can be characterized by an expression level of a hepatocyte cell adhesion molecule 2 (HEPACAM2) biomarker that is higher than a control level of a HEPACAM2 biomarker.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIGS. 1A-D show HEPACAM2 expression in SCLC cells and tumors. (FIG. 1A) HEPACAM2 mRNA levels in cancer cell lines are based on RNA-seq data from the CCLE. (FIG. 1B) HEPACAM2 mRNA levels were validated by qPCR and presented as log 10 of fold change related to β-actin mRNA expression level (symbols show an average of three replicates per cell line). Note: Downloaded from the CCLE dataset found on cBioPortal.org, we used RNA-seq value for each HEPACAM2 level, log 2 (RPKM), to compare our qPCR data. (FIG. 1C) HEPACAM2 mRNA levels in tumor samples were detected by RNA-seq comparing SCLC (n=29) with other cancers in TCGA (BRCA: breast cancer, CR: colorectal cancer, GBM: glioblastoma, LUAD: lung adenoma NSCLC, LUSC: lung squamous NSCLC, PRAD: prostate adenoma cancer, SKCM: skin melanoma cancer). (FIG. 1D) HEPACAM2 mRNA levels in tumor samples were detected by RNA-seq comparing SCLC (n=29) to normal lung (n=25) and both typical (n=17) and atypical (n=13) pulmonary carcinoid tumors.

FIGS. 2A-E show localization protein expression of HEPACAM2. (FIG. 2A) Immunofluorescence staining of HEPACAM2 in SCLC cell lines revealed that most protein expression localizes to the plasma membrane. ProSci Ab was used to detect HEPACAM2 (pseudo green) and counterstained with Hoechst (pseudo blue). (FIG. 2B) Immunofluorescence staining of overexpressed HEPACAM2 (pseudo green) in an NSCLC cell line, H1299, revealed most protein expression localizes to the plasma membrane, as evidenced by the counterstained (pseudo pink) with a plasma membrane (CELLBRITE) marker (far right panel); scale bar, 10 μm. (FIG. 2C) Western blots of identical protein lysates of control (Con) and HEPACAM2 overexpressing (OE) H1299 cells were detected using both FLAG and ProSci antibodies. Black arrows indicate the major ˜70 kDa band. (FIG. 2D) Western blots of HEPACAM2 expression in identical protein lysates of control (C), vehicle-treated (V), and tunicamycin-treated (T) HEPACAM2 overexpressing H1299 cells detected by both FLAG and ProSci antibodies. Black arrows indicate the major ˜70 kDa band, red arrow ˜50 kDa band. (FIG. 2E) Western blots of HEPACAM2 expression in biotin-pulldown control (C) and HEPACAM2 overexpressing (OE) H1299 cells were detected with ProSci antibody after streptavidin pulldown. Black arrow indicates the major ˜70 kDa band;

FIGS. 3A-B show HEPACAM2 expression detected in EVs of SCLC cell lines. (FIG. 3A) mRNAs of HEPACAM2 could be detected in EVs of SCLC cell lines (H526, H209, and H510A) but not NSCLC cell lines (PC9, H1299, HCC15, and A427) and a normal (BEAS2B) cell line. For better visualization, the HEPACAM2 mRNA is the ratio of H2:β-actin log 2 transformed. An Unpaired t-test using Welch's correction was performed using GraphPad Prism version 10.1.0. (FIG. 3B) HEPACAM2 protein was also found via LC-MS/MS in secretomes of NE-specific SCLC cell lines (ASCL1SCLC), more than the current ADC targets for SCLC, such as DLL3 and SEZ6; HBEC-34KT, a normal epithelial cell line of the lung;

FIGS. 4A-C show high HEPACAM2 expression correlates with NE carcinoma in patient tumors. (FIG. 4A) Box plots showing mRNA levels of HEPACAM2 in NSCLC and SCLC. Two-tailed ANOVA test. (FIG. 4B) Spatially-resolved expression of HEPACAM2 (top left) is specific to the region of SCLC histology, expressing high NE genes, i.e., SYP and ASCL1 (bottom panels), but not the NSCLC region with features of adenocarcinoma (i.e., NAPSA top right) in a combined NSCLC/SCLC patient tissue sample; performed by 10× Genomics and image generated using LoupeBrowser 6.4.1. (FIG. 4C) Heatmap of TFs ordered by HEPACAM2 and z-scaled by row, allowing comparison between samples;

FIGS. 5A-D show high HEPACAM2 expression and association with NE carcinoma in patient tumors. In patient samples, no change in HEPACAM2 level of expression was found associated with different stages of disease (FIG. 5A), biopsy locations (FIG. 5B), and patient response (FIG. 5C). High levels of HEPACAM2 expression negatively correlated with PFS and OS in our SCLC cohort (FIG. 5D);

FIG. 6 shows high HEPACAM2 expression in treatment-emergent prostatic small cell carcinoma. A positive trend in HEPACAM2 (left panel), SEZ6 (middle panel), and INSM1 (right panel) mRNA expression as cancer transitions from adenocarcinoma to adenocarcinoma with NE features to small cell carcinoma of the prostate;

FIG. 7 shows high HEPACAM2 expression is not associated with other targets for antibody-drug conjugate therapies. Heatmaps showing the relative expression of some common antibody-drug conjugate targets vs. HEAPACAM2 in two examined SCLC cohorts. Note: The left panel represents late-stage SCLC, vs. early-stage SCLC (right panel);

FIGS. 8A-B show functional effects of HEPACAM2 knockdown in vitro. (FIG. 8A) Western blots of identical protein lysates of control (Con) and HEPACAM2 knockdown (H2-1) H69 cells were detected using the ProSci antibody. Black arrows indicate the major ˜70 kDa band. (FIG. 8B) HEPACAM2 knockdown in H69 cells attenuated the mRNA expression of ASCL1 and Myc;

FIGS. 9A-B show positive correlation between ASCL1 and HEPACAM2. (FIG. 9A) ASCLC1 is positively associated with HEPACAM2 based on Pearson analysis. (FIG. 9B) ENCODE database indicated that ASCL1 binds the HEPACAM2 promoter in several SCLC cell lines;

FIGS. 10A-C show functional effects of HEPACAM2 knockdown in SCLC cells. HEPACAM2 knockdown in H1694 cells resulted in a decrease in cell growth as measured by CCK8 proliferation assay (FIG. 10A), a decrease in migration rate as measured by transwell assay (FIG. 10B), and an increase in cell-cell interaction as indicated by cell attachment counts (FIG. 10C);

FIGS. 11A-B show homology between human HEPACAM2 and HEPACAM. (FIG. 11A) The primary amino acid sequence of the human HEPACAM2 sequence (UniProt A8MVW5) aligned with human HEPACAM (UnitProt Q14CZ8). Regions of homology are highlighted. (FIG. 11B) Cartoon of HEPCAM2 and its regions of homology with HEPACAM is high highlighted;

FIGS. 12A-D show transcriptomic changes caused by HEPACAM2 overexpression. (FIG. 12A) Enrichment pathway analysis revealed shared genes significantly up/down regulated by HEPACAM2 overexpression in A549 and H1299 cells. (FIG. 12B) Venn diagram showing the number of genes in which mRNA expression was significantly (p<0.05) increased (UP) or decreased (DN) by at least two-fold in A549 and H1299 cells by HEPACAM2 overexpression. (FIG. 12C) qPCR validation of MMP2, MMP9 and SPARC expression showing fold-change induced by HEPACAM2 overexpression vs controls (n=3 replicates per cell line). (FIG. 12D) ELISA demonstrates the altered MMP 2 and 9 expressions in control and HEPACAM2 overexpressing (OE) cells (n=1 concentrated supernatant per cell line);

FIG. 13 shows HEPACAM2 expression in Large Cell NE Carcinoma. HEPACAM2 expression is significantly higher in SCLC cell lines than in LCNEC cell lines;

FIG. 14 shows HEPACAM2 expression in normal tissue. Data obtained from the GTEx Portal (https://gtexportal.org/home/) show that compared to other SCLC-specific markers, such as SEZ6, CD276, DLL3 and TACSTD2, HEPACAM2 expression is relatively lower in normal tissue;

FIG. 15 shows combined SCLC tissue. H&E image of combined SCLC tissue depicting multiple entities, including SCLC, LCNEC, and adenocarcinoma;

FIG. 16 shows SCLC cohort with high and low HEPACAM2 expression. Histogram of a SCLC cohort with high vs. low (outliers) HEPACAM2 expression distribution within our cohort of 50 patients. Most samples have an HEPACAM2 expression between 0 and 100. However, we found six outlying samples with “extremely high” HEPACAM2. We used this demarcation to compare the high HEPACAM2 samples to the other 44 samples in the survival analysis;

FIG. 17 shows functional effects of HEPACAM2 SCLC cells. HEPACAM2 knockdown in H69 cells decreased cell growth as measured by CCK8 proliferation assay, P<0.005;

FIGS. 18A-B show NE gene expression in CCLE dataset. Plots of ASCL1 (FIG. 18A), INSM1 (FIG. 18B), SCG3 (FIG. 18B), and DLL3 (FIG. 18A) mRNA expression levels in cancer cell lines were downloaded from the CCLE. Different cancer cell types are indicated below the x-axis along with the number of cell lines tested in parentheses. RNA-seq data is expressed as FPKM (Log2) along the y-axis; and

FIG. 19 shows HEPACAM2 expression in xenograft. H69 cells were inoculated into a dorsal hindlimb of an NRG mouse. Immunofluorescence stained a section of an extracted tumor. ProSci Ab was used to detect HEPACAM2 (pseudo red) and counterstained with DAPI (blue). Yellow arrows indicate plasma membrane signals; scaled bar, 10 μm.

### DETAILED DESCRIPTION

### Definitions

Unless defined otherwise, all technical and scientific terms used herein have the same meaning as is commonly understood by one of skill in the art to which the present disclosure pertains.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

In the context of the present disclosure, the term “about”, when expressed as from “about” one particular value and/or “about” another particular value, also specifically contemplated and disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these aspects are explicitly disclosed.

Optionally, in some aspects, when values or characteristics are approximated by use of the antecedents “about,” “substantially,” or “generally,” it is contemplated that values within up to 15%, up to 10%, up to 5%, or up to 1% (above or below) of the particularly stated value or characteristic can be included within the scope of those aspects.

As used herein, the terms “first,” “second,” etc. should not limit the elements being described by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the terms “optionally” and “optional” can mean that the subsequently described event, circumstance, or material may or may not occur or be present, and that the description includes instances where the event, circumstance, or material occurs or is present and instances where it does not occur or is not present.

As used herein, the terms “subject” and “patient” can be used interchangeably and refer to a vertebrate, such as a mammal (e.g., a human). Mammals can include, but are not limited to, humans, dogs, cats, horses, cows, and pigs.

As used herein, the term “operably linked” can refer to a juxtaposition of components (e.g., polynucleotide or polypeptide domains) such that they are in a relationship permitting them to function in their intended manner. For example, a regulatory sequence is “operably linked” to a coding region when it is joined in such a way that expression of the coding region is achieved under conditions compatible with the regulatory sequence.

As used herein, the term “neuroendocrine carcinoma” can refer to a heterogeneous group of malignant neoplasms that exhibit divergent differentiation along epithelial and neuroendocrine cell lines. Neuroendocrine carcinomas include several malignant neoplasms of varying grade and aggressiveness, including typical (low grade) and atypical carcinoids (intermediate grade), large cell neuroendocrine carcinoma (intermediate grade), and SCLC (high grade). Neuroendocrine carcinomas can start in many different parts of the body, including the lungs, gastrointestinal tract, pancreas, prostate, and head and neck region. There are two main types of neuroendocrine carcinoma based on how the tumor cells look under the microscope: small cell neuroendocrine carcinoma (the cancer cells are small with dark round nuclei and little visible cytoplasm; this type is most commonly found in the lungs, but it can also occur in other organs); and large cell neuroendocrine carcinoma (the cancer cells are larger and more irregular in shape; this type can also start in the lungs or other organs, such as the gastrointestinal tract or bladder). The diagnosis of neuroendocrine carcinoma is usually made by examining a biopsy or surgical specimen under a microscope. A pathologist examines the shape, size, and arrangement of the cells, and checks for features such as the number of cells dividing (mitotic activity) and areas of necrosis. These features help confirm that the tumor is high grade. To support the diagnosis, the pathologist will often perform various immunohistochemistry assays, e.g., to detect specific proteins produced by neuroendocrine cells, such as synaptophysin, chromogranin A, and CD56. Neuroendocrine carcinoma is classified as a high-grade cancer. Neuroendocrine carcinomas can be clinically staged. The stage of a neuroendocrine carcinoma describes how far the cancer has spread in the body. Staging usually takes into account: the size of the tumor; whether cancer cells have spread to nearby lymph nodes; and whether the cancer has spread to other organs. Imaging tests such as CT scans, MRI, or PET scans may be used to determine the stage. A biopsy of nearby lymph nodes or distant sites may also be performed. Neuroendocrine carcinomas can give rise to different cancers, such as small cell lung carcinoma and prostate cancer.

As used herein, the term “small cell lung carcinoma” or “SCLC” can refer to a type of highly malignant cancer that most commonly arises within the lung; although, it can occasionally arise in other body sites, such as the cervix, prostate, and gastrointestinal tract. SCLC is a highly aggressive form of neuroendocrine carcinoma. These cancers are typically described as poorly differentiated cells with a high proliferation rate. While most commonly found in the lungs, neuroendocrine carcinomas such as SCLC can also develop in other areas of the body, including the digestive tract and skin. They may cause symptoms related to abnormal hormone production. SCLC has been divided into two clinicopathological stages, termed limited stage (LS) and extensive stage (ES). The stage is generally determined by the presence or absence of metastases, whether or not the tumor appears limited to the thorax, and whether or not the entire tumor burden within the chest can feasibly be encompassed within a single radiotherapy portal. In general, if the tumor is confined to one lung and the lymph nodes close to that lung, the cancer is said to be LS. If cancer has spread beyond that, it is said to be ES. When SCLC is found with one or more differentiated forms of lung cancer, such as squamous cell carcinoma or adenocarcinoma, the malignant tumor is then diagnosed and classified as a combined small cell lung carcinoma (c-SCLC). Small-cell lung carcinoma can occur in combination with a wide variety of other histological variants of lung cancer, including extremely complex malignant tissue admixtures. See, also, Rudin et al., Nat Rev Dis Primers, 14 Jan. 2021, 71(3), Pgs. 1-43.

As used herein, the term “hepatocyte cell adhesion molecule 2” or “HEPACAM2” can refer to a type I transmembrane glycoprotein in the immunoglobulin superfamily that functions as a mitotic regulator. It is involved in cell adhesion and plays a crucial role in centrosome maturation during prometaphase by translocating to centrosomes after poly(ADP-ribosylation). In one aspect, HEPACAM2 can be encoded by the nucleic acid having SEQ ID NO: 7. In another aspect, HEPACAM2 can have the amino acid sequence of SEQ ID NO: 9. HEPACAM2 is discussed in McColl et al., Cancer, 1 Jan. 2025, Vol. 131, No. 1, e35557.

As used herein, the term “HEPACAM2 biomarker” can refer to a nucleic acid molecule (e.g., a gene or gene fragment) or an expression product thereof (e.g., a polypeptide or peptide fragment or variant thereof) whose differential expression (presence, absence, over-expression or under-expression relative to a reference or control) within a cell or tissue indicates the presence or absence of a neuroendocrine carcinoma, such as SCLC.

As used herein, an “expression product” can refer to a transcribed sense or antisense RNA molecule (e.g., an mRNA), or a translated polypeptide corresponding to or derived from a polynucleotide sequence. In some embodiments, an expression product can refer to an amplification product (amplicon) or cDNA corresponding to the RNA expression product transcribed from the polynucleotide sequence.

As used herein, the terms “differential expression” or “differentially expressed” can refer to a difference in the frequency or quantity, or both, of a HEPACAM2 biomarker in a cell or tissue or sample derived from a subject having a neuroendocrine carcinoma compared to a reference or control cell or tissue or sample (e.g., a cell derived from a subject without cancer or with undetectable cancer or a normal cell derived from a subject who has undergone successful resection of a neuroendocrine carcinoma). In some embodiments, the control or reference cell may be a non-small cell lung carcinoma (NSCLC). In some embodiments, differential expression can refer to a difference in the frequency or quantity, or both, of a HEPACAM2 biomarker in a neuroendocrine carcinoma cell compared to the reference or control cell. For example, differential expression of a HEPACAM2 biomarker can refer to an elevated level or a decreased level of expression of the biomarker in samples of patients diagnosed with a neuroendocrine carcinoma compared to samples of reference or control subjects, e.g., measurement of protein level or antibody titer in blood, urine, saliva, serum, pleural effusions or bronchioalveolar lavages samples taken from neuroendocrine carcinoma patients compared to the measurement of protein level or antibody titer in blood, urine, saliva, serum, pleural effusions or bronchioalveolar lavages samples taken from references or controls, including healthy subjects and subjects with respiratory airway infections like bronchitis and bronchiolitis. Alternatively or additionally, differential expression of a HEPACAM2 biomarker can refer to detection at a higher frequency or at a lower frequency of the biomarker in samples of neuroendocrine carcinoma patients compared to samples of reference or control subjects. A HEPACAM2 biomarker can be differentially present in terms of quantity, frequency or both. In some embodiments, differential expression of a HEPACAM2 biomarker may be measured at different time points, e.g., before and after therapy. By “level of expression” is meant the level of mRNA, as well as pre-mRNA nascent transcript(s), transcript processing intermediates, mature mRNA(s), and degradation products, encoded by a gene in the cell, and/or the level of protein, protein fragments, and degradation products in a cell.

The difference in quantity or frequency or both of a HEPACAM2 biomarker may be measured by any suitable technique, such as a statistical technique. For example, a HEPACAM2 biomarker can be differentially expressed between a neuroendocrine carcinoma sample and a reference or control sample, if the frequency of detecting the HEPACAM2 biomarker in a neuroendocrine carcinoma sample is significantly higher or lower than in the reference or control sample, as measured by standard statistical analyses such as student's t-test, where p<0.05 is generally considered statistically significant. In some embodiments, a HEPACAM2 biomarker is differentially expressed if it is detected at least about 20, 30, 40, 50, 60, 70, 80, 90, 100% or more or 2-, 5-, 10- or more fold more or less frequently in a neuroendocrine carcinoma sample compared to a reference or control sample. Alternatively or additionally, a HEPACAM2 biomarker is differentially expressed if the amount of the HEPACAM2 biomarker in a neuroendocrine carcinoma sample is statistically significantly different, e.g., by at least 20, 30, 40, 50, 60, 70, 80, 90, 100% or more or 2-, 5-, 10- or more fold when compared to the amount of the HEPACAM2 biomarker in a reference or control sample; or if it is detectable in one sample and not detectable in the other. In some embodiments, differential expression may refer to an increase or decrease in expression of at least 20, 30, 40, 50, 60, 70, 80, 90, 100% or more or 2-, 5-, 10- or more fold, in a test sample (e.g., a suspect neuroendocrine carcinoma sample) relative to a reference sample.

A “biological sample” can be any organ, tissue, cell, or cell extract isolated from a subject, such as a sample isolated from a mammal having a neuroendocrine carcinoma or at risk for a neuroendocrine carcinoma (e.g., based on family history or personal history, such a heavy smoking). For example, a biological sample can include, without limitation, cells or tissue (e.g., from a biopsy or autopsy) solid lung tumors, sputum, cough, bronchoalveolar lavage, bronchial brushings, buccal mucosa, peripheral blood, whole blood, red cell concentrates, platelet concentrates, leukocyte concentrates, blood cell proteins, blood plasma, platelet-rich plasma, a plasma concentrate, a precipitate from any fractionation of the plasma, a supernatant from any fractionation of the plasma, blood plasma protein fractions, purified or partially purified blood proteins or other components, serum, tissue or fine needle biopsy samples, and pleural fluid, etc. isolated from a mammal with a neuroendocrine carcinoma, or any other specimen, or any extract thereof, obtained from a patient (human or animal), test subject, healthy volunteer, or experimental animal. A “biological sample” may also include sections of tissues such as frozen sections taken for histological purposes. A “biological sample” may also be a cell or cell line created under experimental conditions, that is not directly isolated from a subject.

As used herein, a “control” or “reference” can refer to a biological sample obtained for use in determining base-line expression or activity, e.g., of HEPACAM2. Accordingly, a control sample may be obtained by a number of means including from non-cancerous cells or tissue, e.g., from cells surrounding a tumor or cancerous cells of a subject; from subjects not having a neuroendocrine carcinoma; from subjects not suspected of being at risk for a neuroendocrine carcinoma; or from cells or cell lines derived from such subjects. A control can also include a previously established standard, such as a previously characterized SCLC, NSCLC including SQC, AC and NSCLC with or without neuroendocrine origin. Accordingly, any test or assay conducted according to the present disclosure may be compared with the established standard and it may not be necessary to obtain a control sample for comparison each time.

As used herein, “increased” or “elevated” or “decreased” can refer to expression level of a HEPACAM2 biomarker in a biological sample as compared to a reference or control level representing the same biomarker or a different biomarker. In certain aspects, the reference or control level may be a reference or control level of expression (e.g., HEPACAM2 expression) from a non-cancerous tissue from the same subject or from a cancerous tissue that is not a neuroendocrine carcinoma. Alternatively, the reference or control level may be a reference or control level of expression (e.g., HEPACAM2 expression) from a different subject or group of subjects. For example, the reference or control level of expression (e.g., HEPACAM2 expression) may be an expression level obtained from a biological sample (e.g., a tissue, fluid or cell sample) of a subject or group of subjects without neuroendocrine carcinoma, with neuroendocrine carcinoma, with a neuroendocrine carcinoma that has not undergone transdifferentiation, or an expression level obtained from a non-cancerous tissue of a subject or group of subjects with neuroendocrine carcinoma. The reference or control level may be a single value or may be a range of values. The reference or control level of expression (e.g., HEPACAM2 expression) can be determined using any method known to those of ordinary skill in the art. The reference or control level may also be depicted graphically as an area on a graph. In certain embodiments, a reference or control level is a normalized level.

As used herein, the term “overall survival” or “OS” can refer to the patient remaining alive for a defined period of time, such as 1 year, 5 years, etc. from the time of diagnosis or treatment of a neuroendocrine carcinoma. In one example, OS can be determined from the date of diagnosis (with a neuroendocrine carcinoma) to the date of death and be censored at the date of the last follow-up for survivors.

As used herein, the term “progression-free survival” or “PFS” can refer to the patient remaining alive, without the neuroendocrine carcinoma progressing or getting worse. In one example, PFS can be determined from the date of diagnosis (with a neuroendocrine carcinoma) to the date of either disease progression or the date of death, whichever occurs first, and be censored at the last follow-up for those (patients) alive without disease progression.

As used herein, the term “treatment” can refer to clinical intervention designed to alter the natural course of the individual or cell being treated during the course of clinical pathology (e.g., a neuroendocrine carcinoma). Desirable effects of treatment include decreasing the rate of disease progression, ameliorating or palliating the disease state, and remission or improved prognosis. For example, a subject is successfully “treated” if one or more symptoms associated with a neuroendocrine carcinoma are mitigated or eliminated, including, but are not limited to, reducing the proliferation of (or destroying) cancerous cells, decreasing symptoms resulting from the disease, increasing the quality of life of those suffering from the disease, decreasing the dose of other medications required to treat the disease, and/or prolonging survival of individuals. In some instances, “treating” a disease such as a neuroendocrine carcinoma refers to delaying progression of the disease, i.e., deferring, hindering, slowing, retarding, stabilizing, and/or postponing development of the disease. This delay can be of varying lengths of time, depending on the history of the neuroendocrine carcinoma and/or individual being treated. As is evident to one skilled in the art, a sufficient or significant delay can, in effect, encompass prevention, in that the individual does not develop the neuroendocrine carcinoma. For example, a late-stage neuroendocrine carcinoma, such as development of metastasis, may be delayed.

As used herein, the term “therapeutically effective amount” can refer to the amount of an agent (e.g., an agent that effectively inhibits or decreases an expression level of a HEPACAM2 biomarker in vivo or ex vivo, as compared to a control level of a HEPACAM2 biomarker) determined to produce any therapeutic response in a subject (e.g., prolong the survivability of the subject, and/or inhibit overt clinical symptoms). Treatments that are therapeutically effective within the meaning of the term as used herein can include treatments that improve a subject's quality of life even if they do not improve the disease outcome per se. Such therapeutically effective amounts are readily ascertained by one of ordinary skill in the art. Thus, to “treat” can mean to deliver such an amount. Thus, treating can prevent or ameliorate any pathological symptoms of a disease or disorder (e.g., a neuroendocrine carcinoma disclosed herein.

As used herein, the term “sequence identity” can refer to the extent to which two optimally aligned polynucleotide or polypeptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, N.J. (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “percent sequence identity” or “percent identity” can refer to the percentage of identical amino acids in an amino acid sequence as compared to a reference polypeptide. The phrase “substantially identical,” or “substantial identity” in the context of two polypeptide or protein sequences, can refer to two or more sequences or subsequences that have at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% amino acid residue identity, when compared and aligned for maximum correspondence, as measured using a known sequence comparison algorithm or by visual inspection.

As used herein, the term “anti-tumor effect” can refer to a biological effect observed when treating solid tumors which manifests in a variety of ways, including, for example, by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the engineered immune effector cells and expression systems of the present disclosure in prevention of the occurrence of tumor in the first place.

As used herein, the term “high-risk”, when used with reference to a subject or population of subjects at risk for developing a neuroendocrine carcinoma (e.g., SCLC) can refer to subjects with specific characteristics that increase their likelihood of developing a neuroendocrine carcinoma (e.g., SCLC) or having a more aggressive form, such as a history of smoking, exposure to carcinogens like asbestos, or certain genetic factors. In one example, a patient (or population of patients) at high risk of developing a neuroendocrine carcinoma (e.g., SCLC) has a history of smoking, including past use as well as current use and extensive history (e.g., high pack-years).

### Overview

As discussed in greater detail below, the inventors surprisingly found that HEPACAM2 has markedly higher expression in SCLC than all other cancers. Biotin-pulldown experiments demonstrated that HEPACAM2 was present on the cell surface, as evidenced by the localization of immunofluorescence signal to the plasma membrane immunofluorescence signal. Knockdown of HEPACAM2 in SCLC cells decreased in ASCL1 and MYC expression, cell proliferation, and migration. Gene Set Enrichment Analyses (GSEA) of HEPACAM2 overexpression demonstrated the dysregulation of the extracellular matrix pathway. In patients with SCLC, it was found that HEPACAM2 expression negatively correlates with progression-free survival (PFS) and overall survival (OS) and has little or no difference between stages of SCLC nor between primary and metastatic sites. In treatment-emergent prostatic small cell carcinoma, HEPACAM2 expression increased with the progression from adenocarcinoma to adenocarcinoma with NE features and NE prostatic carcinoma. Remarkably, the inventors herein provide the first description of HEPACAM2, its remarkable high expression, especially its specificity and overall biology in small cell carcinoma. Given its high level and specificity in all stages of SCLC and its potential to be detected extracellularly, HEPACAM2 advantageously provides a critical diagnostic marker to screen high-risk populations for SCLC for its early presence before metastatic spread and at the beginning of relapse to improve its poor clinical outcome.

### Methods

The present disclosure provides a HEPACAM2 biomarker, e.g., nucleic acid molecules and expression products thereof, that are differentially expressed in neuroendocrine carcinoma cells, compared to normal cells derived from subjects without neuroendocrine carcinoma.

As such, one aspect of the present disclosure can include a method of diagnosing a patient with a neuroendocrine carcinoma based on a detected expression level of a HEPACAM2 biomarker in a biological sample previously obtained from the patient. In one example, the subject can be diagnosed with SCLC, such as early-stage SCLC (e.g., Stage I and/or Stage II, based on the TNM staging system). In another example, the subject can be diagnosed with any one of Stage 0, Stage I, Stage II, Stage III, or Stage IV SCLC (based on the TNM staging system) based on a detected expression level of a HEPACAM2 biomarker in a biological sample previously obtained from the patient. In another example, the subject can be diagnosed with SCLC, such as limited stage (LS) or extensive stage (ES) based on a detected expression level of a HEPACAM2 biomarker in a biological sample previously obtained from the patient.

In some instances, the patient can be a member of a high-risk population for SCLC and is free or substantially free of metastases. In other instances, the patient does not exhibit a high or increased expression level (e.g., a nucleic acid molecule or expression product(s) thereof) of delta-like ligand 3 (DLL3), seizure-related gene 6 (SEZ6) and/or trophoblast cell surface antigen 2 (TROP2) as compared to a control expression level of DLL3, SEZ6 and/or TROP2.

One step of the method for diagnosing a subject with a neuroendocrine carcinoma can include assaying for an expression level of a HEPACAM2 biomarker in a biological sample previously obtained from the patient.

In some instances, the biological sample is assayed for an expression level of a HEPACAM2 biomarker comprising HEPACAM2 mRNA. In one example, the HEPACAM2 mRNA can have the nucleic acid sequence shown by SEQ ID NO: 8, or has at least 80%, 90%, 95%, 96%, 97%, 98%, or 99% identity to the nucleic acid sequence shown by SEQ ID NO: 8.

In other instances, the biological sample is assayed for an expression level of a HEPACAM2 biomarker comprising a HEPACAM2 protein (e.g., a HEPACAM2 protein expressed and localized at a surface of a neuroendocrine carcinoma cell). In one example, the HEPACAM2 protein can have the amino acid sequence of the HEPACAM2 protein is shown by SEQ ID NO: 9, or has at least 80%, 90%, 95%, 96%, 97%, 98%, or 99% identity to the amino acid sequence shown by SEQ ID NO: 9.

In one aspect, one or more assays to determine an expression level of a HEPACAM2 biomarker can be performed on one or more extravesicles secreted by a cell comprising a neuroendocrine carcinoma cell, or a cell suspected of comprising a neuroendocrine carcinoma cell.

Generally speaking, the expression level of a HEPACAM2 biomarker mentioned herein can be determined using procedures done at the protein or the nucleic acid level.

Methods to determine HEPACAM2 biomarker expression level can include: an immunoassay, wherein the HEPACAM2 biomarker expression level is determined using antibodies that specifically bind to a HEPACAM2 protein encoded by a HEPACAM2 gene; a hybridization assay, wherein the HEPACAM2 biomarker expression level is determined using a probe that hybridizes to the nucleic acid molecule(s) encoding said gene; immunohistochemistry, wherein the HEPACAM2 biomarker expression level is used for in vitro diagnosis/sub-typing and staging of a neuroendocrine carcinoma; or imaging, wherein the HEPACAM2 biomarker expression level is used for in vivo diagnosis, monitoring of disease progression or treatment response (using, e.g., computed tomography (CT), positron emission tomography (PET) and/or PET-CT).

In one example, extravesicles (or EVs) can be isolated from a biological sample according to the exosome isolation particle described in the Example below. The isolated EVs can then be assay for a HEPACAM2 biomarker expression level using one more assays, e.g., as described below.

In one example, PCR (e.g., qPCR) can be used to assay the expression level of the HEPACAM2 mRNA in a biological sample, e.g., a biological sample comprising one or more extravesicles secreted by a cell comprising a neuroendocrine carcinoma cell, or a cell suspected of comprising a neuroendocrine carcinoma cell.

In another example, immunofluorescence microscopy or a biotin-pulldown technique can be used to assay the expression level of the HEPACAM2 protein in a biological sample, e.g., a biological sample comprising one or more extravesicles secreted by a cell comprising a neuroendocrine carcinoma cell, or a cell suspected of comprising a neuroendocrine carcinoma cell.

The biotin pull-down technique is an affinity-based method to identify molecules (like proteins or nucleic acids) that bind to a known “bait” molecule, which is typically labeled with biotin. A biotinylated bait is first immobilized onto streptavidin-coated beads, and then incubated with a sample containing potential binding partners (the “prey”). After washing away unbound molecules, the bait-prey complex is isolated, and the prey is then released for analysis, often by mass spectrometry. Steps of the technique can be as follows:
bait preparation—the molecule of interest (the bait), such as a protein or RNA, is labeled with a biotin tag;
immobilization—the biotinylated bait is then mixed with streptavidin-coated beads;
incubation with lysate—the prepared bait-bead complex is incubated with a sample containing potential interacting proteins or other molecules (the prey), such as a cell lysate;
washing—the complex is washed to remove any unbound molecules and other non-specific interactions; and
elution and analysis—the interacting “prey” molecules are released (eluted) from the bait and beads. This complex mixture is then analyzed, often by mass spectrometry (MS), to identify the prey molecule(s).

In another aspect, the expression level of the HEPACAM2 biomarker, as detected by the assay(s), can be compared to a control expression level of a HEPACAM2 biomarker. In such instances, a detected expression level of the HEPACAM2 biomarker that is higher than a control expression level of a HEPACAM2 biomarker can be indicative of a neuroendocrine carcinoma in the patient. As discussed below, one or more therapeutic regimens can be administered to such patients.

Another aspect of the present disclosure can include a method of treating a patient having, or suspected of having, a neuroendocrine carcinoma, e.g., wherein the neuroendocrine carcinoma is characterized by an expression level of a HEPACAM2 biomarker that is higher than a control level of a HEPACAM2 biomarker. One step of the method can comprise administering a therapeutically effective amount of a therapeutic regimen to the patient.

In certain aspects, the therapeutic regimen can be a therapy selected from the group consisting of chemotherapy, targeted therapy, radiation therapy, immunotherapy, surgery, and combinations thereof.

In one example, the chemotherapy can include administering one or a combination of pharmaceutical agents including, but not limited to, etoposide, cisplatin or carboplatin, cyclophosphamide, doxorubicin, ifosfamide, and irinotecan. The amount of the pharmaceutical agent delivered to the patient may be variable. In one suitable embodiment, the pharmaceutical agent may be administered in an amount effective to cause arrest or regression of the neuroendocrine carcinoma in a host.

In another example, the targeted therapy can include administering an antibody-drug conjugate (ADC) and/or RNA interfering modality. ADCs consist of monoclonal antibodies that specifically target cancer cells and are attached to cytotoxic drugs, allowing for targeted killing of cancer cells while sparing healthy tissues. In such instances, an ADC according to the present disclosure can comprise an antibody (e.g., monoclonal antibody, such as those described in the Example herein) that specifically binds a HEPACAM2 antigen or epitope and which is conjugated to a known chemotherapeutic agent, such as those discussed above. An RNA interfering modality can include a single or double-stranded RNA consisting of a strand having a sequence complementary to the mRNA of a target gene (e.g., HEPACAM2) and a strand having a sequence complementary thereto that is capable of inducing the degradation of the mRNA of the target gene to thereby inhibit or reduce the expression of the target gene. One example of a RNA interfering modality can include a small interfering RNA or siRNA, which comprises a short dsRNA that mediates efficient gene silencing in a sequence-specific manner.

In another example, the radiation therapy can comprise radiation, such as ionizing radiation. The term “ionizing radiation” or “IR” can refer to radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

In some embodiments, the amount of ionizing radiation is greater than 20 Gy and is administered in one dose. In some embodiments, the amount of ionizing radiation is 18 Gy and is administered in three doses. In some embodiments, the amount of ionizing radiation is at least, at most, or exactly 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, or 40 Gy (or any derivable range therein). In some embodiments, the ionizing radiation is administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some embodiments, the amount of IR may be presented as a total dose of IR, which is then administered in fractionated doses. For example, in some embodiments, the total dose is 50 Gy administered in 10 fractionated doses of 5 Gy each. In some embodiments, the total dose is 50-90 Gy, administered in 20-60 fractionated doses of 2-3 Gy each. In some embodiments, the total dose of IR is at least, at most, or about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 (or any derivable range therein). In some embodiments, the total dose is administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein. In some embodiments, at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some embodiments, at least, at most, or exactly 1, 2, 3, 4, 5, 6, 7, 8, 9, 10, 11, or 12 (or any derivable range therein) fractionated doses are administered per day. In some embodiments, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

In another example, the administered therapeutic regimen can comprise immunotherapy, e.g., a cancer immunotherapy. Cancer immunotherapy (sometimes called immuno-oncology, abbreviated “IO”) is the use of the immune system to treat cancer. Immunotherapies can be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g., carbohydrates). Active immunotherapy directs the immune system to attack tumor cells by targeting TAAs. Passive immunotherapies enhance existing anti-tumor responses and include the use of monoclonal antibodies, lymphocytes and cytokines. Some immunotherapies useful in the methods of the present disclosure are described below and can include, for example, antibodies (e.g., atezolizumab or durvalumab) immune checkpoint inhibitors, inhibition of co-stimulatory molecules, dendritic cell therapy, CAR-T cell therapy, bi-specific T-cell engagers, cytokine therapy, adoptive T-cell therapy, and oncolytic virus therapy.

In some embodiments, the therapeutic regimen comprises CAR-T cell therapy. Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signaling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

In one example, CAR-T therapy can include a CAR-T cell having an antigen binding domain that specifically binds to a HEPACAM2 epitope.

In another example, the therapeutic regimen can comprise surgery. Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatments described herein. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

In another aspect, the therapeutic regimen (e.g., a targeted therapy, a chemotherapy, or an immunotherapy) can be administered subsequent to the beginning of a relapse episode. The term “relapse episode” can be used interchangeably with “cancer recurrence” and refer to a clinical manifestation of parameters of the cancer(s) after a disease-free survival time of the patient, such as the occurrence of tumor tissue and/or metastases and/or a detected expression level of the HEPACAM2 biomarker that is higher than a control level of a HEPACAM2 biomarker. In one example, the therapeutic regimen can be administered subsequent to the beginning of a relapse episode in a patient who was previously diagnosed with, and treated for, a neuroendocrine carcinoma (e.g., SCLC) and whom had some period of disease-free survival time following the diagnosis and treatment prior to the relapse episode.

Another aspect of the present disclosure can include a method of determining a survival prognosis in a patient diagnosed with a neuroendocrine carcinoma. The method can include the following steps: assaying an expression level of a HEPACAM2 biomarker in a biological sample previously obtained from the patient; and calculating a survival prognosis, based on the assayed expression level of the HEPACAM2 biomarker, comprising an overall survival (OS) and/or a progression-free survival (PFS).

Methods for assaying an expression level of a HEPACAM2 biomarker in a biological sample previously obtained from the patient are discussed above.

Calculating a survival prognosis, based on the assayed expression level of the HEPACAM2 biomarker, comprising an OS and/or a PFS can be performed. Methods for determining OS and/or PFS are described, for example, by Ardeshir-Larijani et al., Clinical Lung Cancer, July 2018, Vol. 19, No. 4, Pgs. e489-e501 and Dowlati et al., Ann Oncol., 22 Jan. 2016, Vol. 27, No. 4, Pgs. 642-647.

In certain aspects, a detected expression level of the HEPACAM2 biomarker that is higher than a control level of a HEPACAM2 biomarker can be indicative of a lesser OS and/or a lesser PFS as compared to a control patient. In such instances, a patient determined to have a lesser OS and/or a lesser PFS can be administered a therapeutic regimen, as discussed above.

### Compositions

Another aspect of the present disclosure can include a composition comprising an agent that effectively inhibits or decreases an expression level of a HEPACAM2 biomarker in vivo or ex vivo (e.g., in vitro), as compared to a control level of a HEPACAM2 biomarker.

In one example, the agent can effectively inhibit or decrease the expression level of a HEPACAM2 biomarker in a neuroendocrine carcinoma cell.

In another example, the agent can effectively inhibit or decrease HEPACAM2 mRNA expression, such as a RNA interfering modality (discussed above). Methods for preparing siRNAs to treat SCLC, for example, are disclosed in U.S. Patent Pub. No. US 2009/0317392 A1.

In another example, the agent can comprise a CAR-T cell having an antigen binding domain that specifically binds to a HEPACAM2 epitope. Such CAR-T cells can be prepared according, e.g., to U.S. Pat. Pub. No. 2022/0249563 A1.

In another example, the agent can comprise an ADC (as discussed above). Methods for making ADCs are disclosed, for example, in PCT Pub. No. WO 2018/156634 A1.

In some instances, the agent can be formulated with a pharmaceutically acceptable carrier. Pharmaceutically acceptable carriers can include any and all solvents, dispersion media, coatings, antibacterial and antifungal agents, isotonic and absorption delaying agents, and the like that are physiologically compatible. Preferably, the carrier is suitable for intravenous, intramuscular, subcutaneous or parenteral administration (e.g., by injection). Excipients can include pharmaceutically acceptable stabilizers and disintegrants.

The agents of the present disclosure can be administered by the same route of administration or by different routes of administration. In some embodiments, the therapeutic regimen is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of neuroendocrine carcinoma to be treated, severity and course of the disease, the clinical condition of the patient, the patient's clinical history and response to the treatment, and the discretion of the attending physician.

The treatments disclosed herein may include various “unit doses”. Unit dose is defined as containing a predetermined-quantity of a therapeutic composition, e.g., including an agent disclosed herein. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In practice, and in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of a composition of the present disclosure is one which can provide a blood level of about 1 μM to 150 μM. In another embodiment, the effective dose provides a blood level of about 4 μM to 100 M.; or about 1 μM to 100 μM; or about 1 μM to 50 μM; or about 1 μM to 40 μM; or about 1 μM to 30 μM; or about 1 μM to 20 μM; or about 1 μM to 10 M; or about 10 UM to 150 μM; or about 10 UM to 100 UM; or about 10 UM to 50 μM; or about 25 μM to 150 UM; or about 25 M to 100 μM; or about 25 UM to 50 UM; or about 50 UM to 150 UM; or about 50 UM to 100 μM (or any range derivable therein). In other embodiments, the dose can provide the following blood level of the agent that results from the agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the agent that is administered to a subject is metabolized in the body to a metabolized therapeutic agent, in which case the blood levels may refer to the amount of that agent. Alternatively, to the extent the agent is not metabolized by a subject, the blood levels discussed herein may refer to the unmetabolized agent.

Precise amounts of the composition of the present disclosure also depend on the judgment of the practitioner and are peculiar to each patient. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

Doses for humans or other mammals can be determined without undue experimentation by the skilled artisan, from this disclosure, the documents cited herein, and the knowledge in the art. The dose of the agent(s) and compositions thereof appropriate to be used in accordance with various aspects of the present disclosure will depend on numerous factors. The parameters that will determine optimal doses to be administered for primary and adjunctive therapy generally will include some or all of the following: the disease or condition being treated and its stage; the species of the subject, their health, gender, age, weight, and metabolic rate; the patient's co-morbidities (if any); other therapies being administered; and expected potential complications from the patient's history or genotype. Additional parameters include co-administration with other factors (such as growth factors and cytokines).

### Kits

Another aspect of the present disclosure can include a kit comprising: one or more reagents to determine an expression level of a HEPACAM2 biomarker in a biological sample; and written instructions for use of the kit in detection, subtyping, diagnosis and/or treatment of a neuroendocrine carcinoma.

In certain aspects, a kit of the present disclosure can include one or more reagents corresponding to the HEPACAM2 biomarkers described herein, e.g., antibodies that specifically bind the biomarkers secreted as antigens in body fluids, recombinant proteins that bind biomarker-specific antibodies, nucleic acid probes or primers that hybridize to the biomarkers, etc. In some embodiments, a kit of the present disclosure can include a plurality of reagents, e.g., on an array, corresponding to the HEPACAM2 biomarkers described herein. The kit can include detection reagents, e.g., reagents that are detectably labeled. The kit can include written instructions for use of the kit, e.g., according to the methods described herein, and may include other reagents and information such as control or reference standards, wash solutions, analysis software, etc.

Certain aspects of the present disclosure can include a kit containing compositions of the present disclosure or compositions to implement methods of the present disclosure. In some embodiments, a kit can be used to evaluate one or more HEPACAM2 biomarkers. In certain embodiments, a kit can contain, contains at least, or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein. In some embodiments, there are kits for evaluating HEPACAM2 biomarker expression in a cell (e.g., a neuroendocrine carcinoma cell, either in vivo or in vitro).

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

In certain aspects, negative and/or positive control nucleic acids, probes, and inhibitors are included in some kit embodiments.

### Exemplary Aspects

In view of the described compositions and methods and variations thereof, herein below are certain more particularly described aspects of the present disclosure. These particularly recited aspects should not, however, be interpreted to have any limiting effect on any different claims containing different or more general teachings described herein, or that the “particular” aspects are somehow limited in some way other than the inherent meanings of the language literally used therein.

Aspect 1: A method of diagnosing a patient with a neuroendocrine carcinoma, comprising assaying for an expression level of a hepatocyte cell adhesion molecule 2 (HEPACAM2) biomarker in a biological sample previously obtained from the patient, wherein a detected expression level of the HEPACAM2 biomarker that is higher than a control level of a HEPACAM2 biomarker is indicative of a neuroendocrine carcinoma in the patient.

Aspect 2: The method of Aspect 1, wherein the neuroendocrine carcinoma is small cell lung carcinoma (SCLC).

Aspect 3: The method of any one of Aspects 1-2, wherein the SCLC is early-stage SCLC.

Aspect 4: The method of any one of Aspects 1-3, wherein the patient is a member of a high-risk population for SCLC and is free of metastases.

Aspect 5: The method of any one of Aspects 1-4, wherein the patient does not exhibit a high expression level of DLL3, SEZ6 and/or TROP2 as compared to a control expression level of DLL3, SEZ6 and/or TROP2.

Aspect 6: The method of any one of Aspects 1-5, wherein the expression level of a HEPACAM2 biomarker is expression of a HEPACAM2 mRNA.

Aspect 7: The method of any one of Aspects 1-6, wherein the nucleic acid sequence of the HEPACAM2 mRNA is shown by SEQ ID NO: 8, or has at least 80%, 90%, 95%, 96%, 97%, 98%, or 99% identity to the nucleic acid sequence shown by SEQ ID NO: 8.

Aspect 8: The method of any one of Aspects 1-7, wherein the expression level of a HEPACAM2 biomarker is a HEPACAM2 protein.

Aspect 9: The method of any one of Aspects 1-8, wherein the HEPACAM2 protein is expressed and localized at a surface of a neuroendocrine carcinoma cell.

Aspect 10: The method of any one of Aspects 1-9, wherein the amino acid sequence of the HEPACAM2 protein is shown by SEQ ID NO: 9, or has at least 80%, 90%, 95%, 96%, 97%, 98%, or 99% identity to the amino acid sequence shown by SEQ ID NO: 9.

Aspect 11: The method of any one of Aspects 1-10, wherein the assay is performed on one or more extravesicles secreted by a cell comprising a neuroendocrine carcinoma cell or a cell suspected of comprising a neuroendocrine carcinoma cell.

Aspect 12: The method of any one of Aspects 1-11, wherein qPCR is used to assay the expression level of the HEPACAM2 mRNA.

Aspect 13: The method of any one of Aspects 1-12, wherein immunofluorescence microscopy or a biotin-pulldown technique is used to assay the expression level of the HEPACAM2 protein.

Aspect 14: The method of any one of Aspects 1-13, further comprising administering a therapeutically effective amount of a therapeutic regimen to a patient diagnosed with a neuroendocrine carcinoma.

Aspect 15: The method of any one of Aspects 1-14, wherein the therapeutic regimen is a therapy selected from the group consisting of chemotherapy, targeted therapy, radiation therapy, immunotherapy, surgery, and combinations thereof.

Aspect 16: The method of any one of Aspects 1-15, wherein the targeted therapy comprises an antibody-drug conjugate.

Aspect 17: The method of any one of Aspects 1-16, wherein the immunotherapy comprises a CAR-T cell therapy.

Aspect 18: The method of any one of Aspects 1-17, wherein the targeted therapy comprises a RNA interfering modality.

Aspect 19: The method of any one of Aspects 1-18, wherein the therapeutic regimen is administered subsequent to the beginning of a relapse episode.

Aspect 20: A method of determining a survival prognosis in a patient diagnosed with a neuroendocrine carcinoma, the method comprising: assaying an expression level of a hepatocyte cell adhesion molecule 2 (HEPACAM2) biomarker in a biological sample previously obtained from the patient; and calculating a survival prognosis, based on the assayed expression level of the HEPACAM2 biomarker, comprising an overall survival (OS) and/or a progression-free survival (PFS); wherein a detected expression level of the HEPACAM2 biomarker that is higher than a control level of a HEPACAM2 biomarker is indicative of a lesser OS and/or a lesser PFS as compared to a control patient.

Aspect 21: The method of Aspect 20, wherein the neuroendocrine carcinoma is small cell lung carcinoma (SCLC).

Aspect 22: The method of any one of Aspects 20-21, wherein the patient is diagnosed with Stage 0, Stage I, Stage II, Stage III, or Stage IV SCLC, based on the TNM staging system.

Aspect 23: A composition comprising an agent that effectively inhibits or decreases an expression level of a hepatocyte cell adhesion molecule 2 (HEPACAM2) biomarker in vivo or ex vivo, as compared to a control level of a HEPACAM2 biomarker.

Aspect 24: The composition of Aspect 23, wherein the agent effectively inhibits or decreases HEPACAM2 mRNA expression.

Aspect 25: The composition of any one of Aspects 23-24, wherein the agent comprises a RNA interfering modality.

Aspect 26: The composition of any one of Aspects 23-25, wherein the agent comprises a CAR-T cell having an antigen binding domain that specifically binds to a HEPACAM2 epitope.

Aspect 27: The composition of any one of Aspects 23-26, further comprising a pharmaceutically acceptable carrier.

Aspect 28: The composition of any one of Aspects 23-27, wherein the agent effectively inhibits or decreases the expression level of a hepatocyte cell adhesion molecule 2 (HEPACAM2) biomarker in a neuroendocrine carcinoma cell.

Aspect 29: A method of treating a patient having, or suspected of having, a neuroendocrine carcinoma, the method comprising administering a therapeutically effective amount of a therapeutic regimen to the patient, wherein the neuroendocrine carcinoma is characterized by an expression level of a hepatocyte cell adhesion molecule 2 (HEPACAM2) biomarker that is higher than a control level of a HEPACAM2 biomarker.

Aspect 30: The method of Aspect 29, wherein the therapeutic regimen is a therapy selected from the group consisting of chemotherapy, targeted therapy, radiation therapy, immunotherapy, surgery, and combinations thereof.

Aspect 31: The method of any one of Aspects 29-30, wherein the targeted therapy comprises an antibody-drug conjugate.

Aspect 32: The method of any one of Aspects 29-31, wherein the immunotherapy comprises a CAR-T cell therapy.

Aspect 33: The method of any one of Aspects 29-32, wherein the targeted therapy comprises a RNA interfering modality.

Aspect 34: The method of any one of Aspects 29-33, wherein the therapeutic regimen is administered subsequent to the beginning of a relapse episode.

Aspect 35: The method of any one of Aspects 29-34, wherein the neuroendocrine carcinoma is small cell lung carcinoma (SCLC).

Aspect 36: The method of any one of Aspects 29-35, wherein the patient is diagnosed with Stage 0, Stage I, Stage II, Stage III, or Stage IV SCLC, based on the TNM staging system.

Aspect 37: A kit comprising: one or more reagents to determine an expression level of a hepatocyte cell adhesion molecule 2 (HEPACAM2) biomarker in a biological sample; and written instructions for use of the kit in detection, subtyping, diagnosis and/or treatment of a neuroendocrine carcinoma.

Aspect 38: The kit of Aspect 37, wherein the written instructions recite the method of any one of Aspect 1, Aspect 20 or Aspect 29.

The following Example is for the purpose of illustration only and is not intended to limit the scope of the claims, which are appended hereto.

### Example

In this Example, the inventors identify, for the first time, HEPACAM2 as a gene that is specifically and highly expressed in SCLC relative to all other cancers. The specificity of HEPACAM2 for SCLC relative to all other cancers in the CCLE databases is greater than that of the three neuroendocrine (NE) genes (ASCL1, INSM1, SCG3) and DDL3, which demonstrated more significant enrichment in SCLC vs. NSCLC. More compellingly, many of the non-SCLC cell lines in the CCLE that express high levels of HEPACAM2 are also annotated as having “small cell” characteristics. Therefore, HEPACAM2 expression represents the most specific marker of small cell histology identified to date, regardless of the tissue of origin or stages of the disease.

### Materials and Methods

### Cell Culture: Cell Lines and Reagents

All cell lines were purchased from the American Type Culture Collection (ATCC). Cells were grown in a medium recommended by the supplier. Cells were periodically validated by short tandem repeat profiling. We used the CellPlayer NucLight Red lentivirus (Essen Bioscience) to generate RFP stable cell lines using an MOI of 3. After 48 hr, 0.5 g/ml puromycin was added for selection, and mixed populations of resistant cells expanded.

Lentivirus vector production, concentration, and generation of stable cell lines Lentiviral encoding C-terminal, FLAG-tagged HEPACAM2 and its empty control pReceiver-158 were purchased from GeneCopoeia (NM_001039372.3). Since the predicted N-terminal signal sequenced of HEPACAM2 may be disrupted by a peptide tag or make the tag susceptible to removal during the post-translational process, a FLAG-tag was placed at the C-terminus was that HEPACAM2 has a predicted N-terminal signal sequence that may be disrupted by the FLAG tag or make the tag susceptible to removal during co-translational processing. Three cell lines were transduced (A549, H1299, and H841) with an MOI of 5, and mixed populations resistant to 1 mg/ml G-418 were expanded.

For HEPACAM2 knockdown, SCLC cells with stable HEPACAM2 knockdown was obtained using Genscript's CRISPR Guide 4 in pLentiCrispr V2 against the human genome sequence ENST00000453812.2. H1694 and H69 cells were transduced with lentivirus, and mixed populations were selected with 0.5 μg/ml puromycin. 0.5 g/ml puromycin.

RNA-Seq Analyses and q-PCR

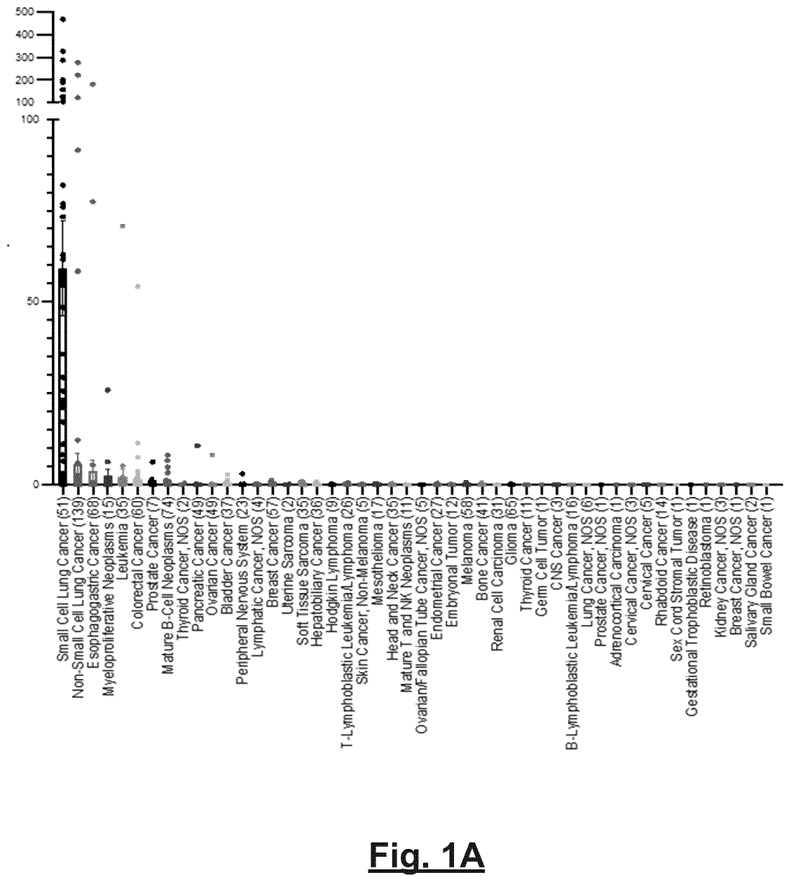

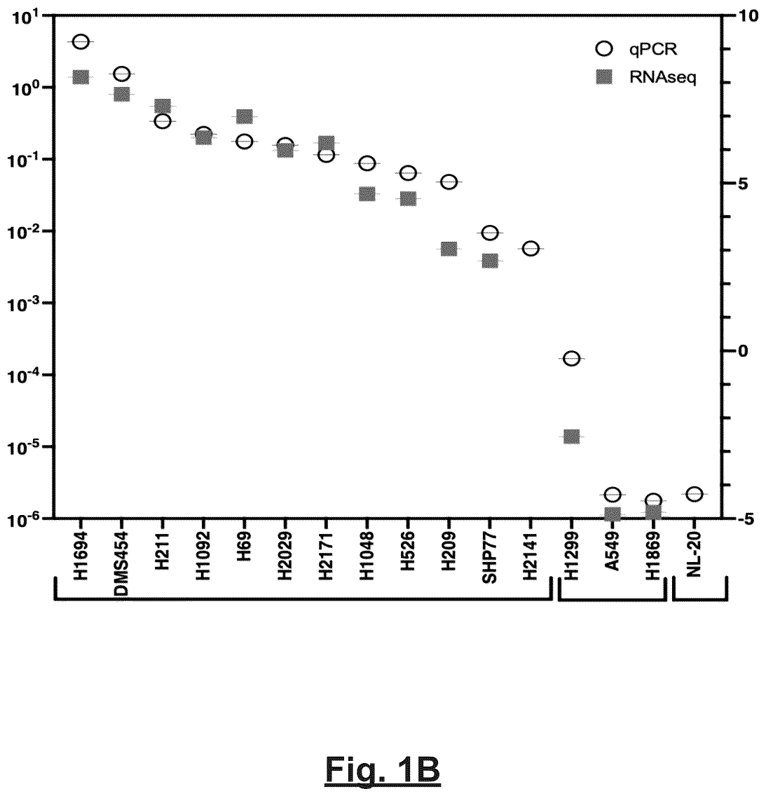

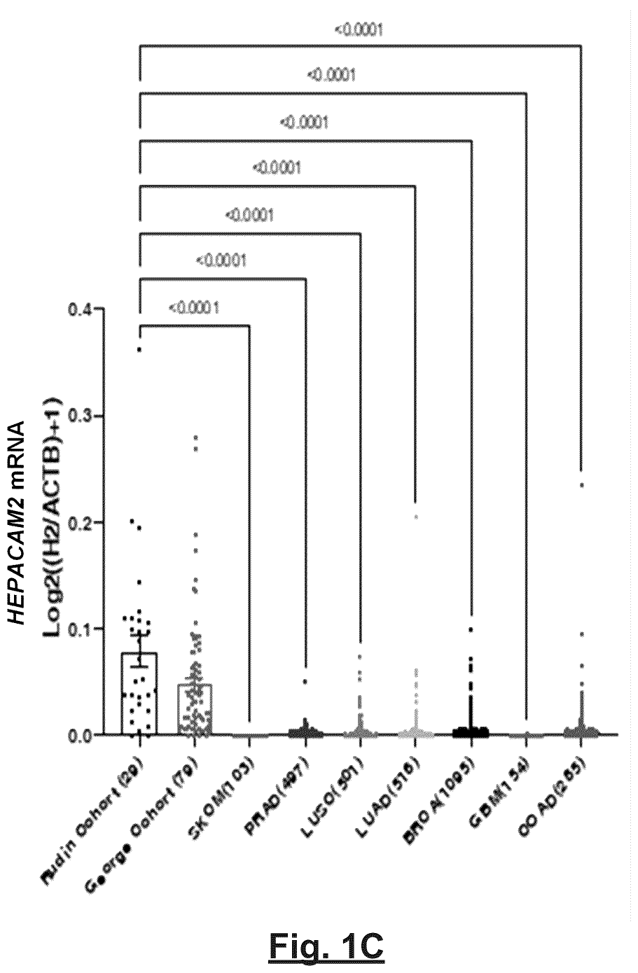

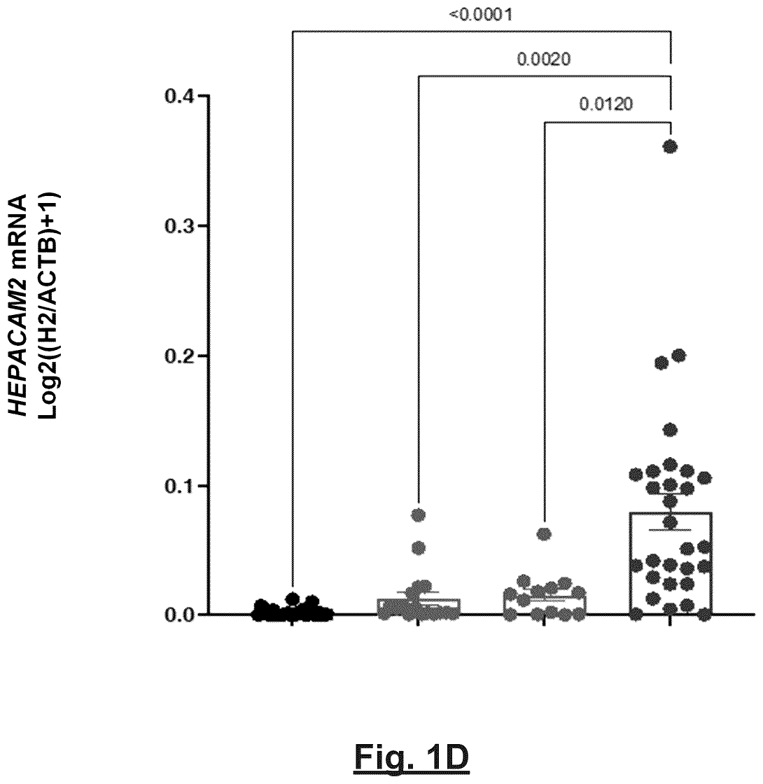

The available CCLE RNA-seq expression data was downloaded from http://broadinstitute.org/ccle to obtain FIG. 1A. Typical and atypical carcinoid RNA-seq raw data files were downloaded from the Gene Expression Omnibus (GEO, accession number GSE118131). Cell line RNA for qPCR experiments was isolated using the Qiagen RNeasy Kit, and 500 ng of RNA was reverse transcribed using the High-Capacity cDNA Reverse Transcription Kit (Thermo Fisher). The resulting cDNAs for measuring endogenous Hepacam2 were amplified using Universal TaqMan master mix and primer/probe sets specific for the genes of interest (Applied Biosystems) before being performed on a Lightcycler 480 II. The probe sets used were: Hs01650957_m1 (HEPACAM2) and Hs00404147_m1 (HEPACAM); with 4333762F (ACTB) was used for normalization. cDNAs for measuring Hepacam2 knockdown cells were amplified with SyBr Green and the following primer sets: Hepacam2 Forward:

Hepacam2
Forward:
(SEQ ID NO: 1)
CTCTACCTACCCGTCCACTATG,
Reverse:
(SEQ ID NO: 2)
TGTGGGGTCTCTCAAATAGCC;
ASCL1
Forward:
(SEQ ID NO: 3)
CGCGGCCAACAAGAAGATG;
Reverse:
(SEQ ID NO: 4)
CGACGAGTAGGATGAGACCG,
Myc
Forward:
(SEQ ID NO: 5)
GGTGCTCCATGAGGAGACA,
Reserve:
(SEQ ID NO: 6)
CCTGCCTCTTTTCCACAGAA.

SCLC tumor RNA-seq data in the study by Rudin et al. ((2012) Comprehensive genomic analysis identifies SOX2 as a frequently amplified gene in small-cell lung cancer. Nat Genet 44, 1111-1116) was downloaded from the European Genome database and analyzed as previously described (McColl et al., (2017) Reciprocal expression of INSM1 and YAP1 defines subgroups in small cell lung cancer. Oncotarget 8, 73745-73756) to obtain FIG. 1C. Briefly, Tophat was used to do the alignment; then Cufflinks was employed to obtain the FPKM (fragments per kilobase per million) values. The transcriptome files of the other cancers (breast: BRCA, colorectal adenocarcinoma: COAD, brain glioblastoma: GBM, lung adenocarcinoma: LUAD, lung squamous cell carcinoma: LUSC, prostate: PRAD and skin: SKCM) were downloaded from the Cancer Genome Atlas (TCGA) data portal (https://gdc.cancer.gov/). The FPKM values were extracted from those files, and boxplots comparing expression values were generated with R, version 3.2.2.

### RNA-Seq and Data Analysis

RNA-seq was performed using Qiagen kit-purified RNA. Integrity and quantification of RNA was assessed using an Agilent 2100 Bioanalyzer (Agilent Technologies, Santa Clara, CA). All the samples have RIN score ≥9.0. Three independent biological replicates were used for RNA-seq. After the QC, mRNA was enriched using oligo (dT) beads. Strand-specific libraries were prepared using the Illumina TruSeq stranded RNA library prep kit (Illumina, San Diego, CA) according to the manufacturer's protocols. Sequencing was performed using Illumina NovaSeq platforms with paired-end 150 bp sequencing format. The raw reads were checked for quality using FastQC and the adapter sequences were trimmed using TraimGalore v0.6.7 (https://github.com/FelixKrueger/TrimGalore). The raw data was then mapped to the GENCODE version 44 hg38 genome. Transcript abundances were quantified using RSEM v1.3.3. Differential gene expressions were performed on the genes using the DESeq2 R package in R 4.2.2 with an adjusted P-value <0.05 considered statistically significant. Pathway analysis was performed using ClusterProfiler. RNA-seq data have been deposited at the Gene Expression Omnibus (GEO) under accession number GSE149035. RNA-seq data from GEO accession number GSE118131, as reported by Laddha et al. ((2019) Integrative Genomic Characterization Identifies Molecular Subtypes of Lung Carcinoids. Cancer Res 79, 4339-4347), was analyzed as described above.

### Protein Lysate Preparation, Western Blotting, and Biotinylation

Whole-cell lysates were prepared as described previously (McColl et al., (2017) Reciprocal expression of INSM1 and YAP1 defines subgroups in small cell lung cancer. Oncotarget 8, 73745-73756) and analyzed using 4-20% Criterion gels (Bio-Rad). Antibodies were purchased from ProSci (HEPACAM2, #7111) and Sigma (Actin, #A5441, and Flag, #F1804).

### Biotinylation

A Cell Surface Protein Isolation Kit (Pierce 89881) was used according to directions. Briefly, 40 million cells were labeled with Sulfo-NHS-SS-Biotin while in culture, lysed with a mild detergent, sonicated, and then incubated with NeutrAvidin Agarose for an hour. Complexes were washed extensively, eluted from the beads, and western blotted as indicated.

### Tunicamycin Treatment

Tunicamycin was purchased from Sigma (TXFR7765). Stocks were made in DMSO, and cells were treated for 24 hr with a 1.0 μg/ml concentration. Cells were processed for western blotting as described above.

### Immunofluorescence and Confocal Microscopy

To investigate the localization of HEPACAM2, we plated 2×10⁵ cells overnight on 35 mm glass-bottom microwell dishes. Cells were washed 3× with Hanks-Buffered Saline Solution (HBSS), fixed for 10 min in 4% methanol-free formaldehyde, washed 3× with HBSS, permeabilized for 10 min with 0.2% Triton X-100 and washed 3× with Phosphate-Buffered Saline with Tween 20 (PBS-T). Cells were blocked for one hr with normal goat serum and incubated overnight at 4° C. with HEPACAM2 Antibody (0.02 μg/μl; ProSci, #7111). Cells were washed 3× with PBS-T, incubated with goat anti-rabbit Alexa 488 for 1 hr, washed 3× with PBS-T, and incubated with Hoechst dye for 10 min. Fluorescence images were acquired using an Olympus BX-60 upright microscope and a Leica DMI 6000B epifluorescence microscope or an Olympus FV1200 IX-83 confocal microscope equipped with an UPlanSApo 60× objective, N.A. 1.35 oil.

### EVs Isolation and Characterization

All the tested cell lines were seeded in 3× T175 flasks. At 70% confluency, the cells were washed in PBS and starved with FBS-depleted media without FBS for 48 hr. EVs were isolated from 80 ml of cell-conditioned media following the exosome isolation protocol modified by Thery et al. ((2006) Isolation and characterization of exosomes from cell culture supernatants and biological fluids. Curr Protoc Cell Biol Chapter 3, Unit 3 22). The cell-conditioned media was centrifuged at 300×g, 4° C. for 10 min to remove living cells. The supernatant was then spun at 2000×g, 4° C. for 10 minutes to remove dead cells. A third centrifugation of the supernatant was performed at 10,000×g, 4° C. for 30 minutes to remove cell debris. The supernatant was filtered through a 0.2 μm filter and ultracentrifuged at 100,000×g, 4° C. for 70 min. The pellet containing the isolated EVs was washed in PBS and ultracentrifuged a second time at 100,000×g, 4° C. for 70 minutes. EVs were resuspended in 100 μl of PBS 1× with 10 μl used for EV characterization by Nanoparticle Tracking Analysis (NTA). NTA analysis was performed using the NanoSight NS300 system (Malvern, Great Malvern, UK) as previously described (Romano et al., (2020) MIR-124a Regulates Extracellular Vesicle Release by Targeting GTPase Rabs in Lung Cancer. Front Oncol 10, 1454).

RNA Extraction from EVs and qRT-PCR

Seventy μl of EVs were used for RNA purification using RNA Cleanup and Concentration Kit (Norgen-Thorold, ON, Canada) following the manufacturer's instructions. The concentration of the purified RNA was determined by Nanodrop, and 200 ng of RNA were retrotranscribed using High-Capacity cDNA Reverse Transcription Kit (Applied Biosystem). qRT-PCR was performed using TaqMan® reagents (TaqMan™ Fast Advanced Master Mix, Thermo Fisher). The probes used were obtained from TaqMan® (#4331182-HEPACAM2 ID: Hs01650957_m1; β-Actin ID: Hs99999903_m1). All the assays were carried out in three technical replicates. Technical replicates in which the Ct value was >38 or below detection were adjusted to 40 for graphical purposes. β-actin expression was used as a housekeeping gene, and relative gene expression was calculated by the 2\hat{ }(−Δct) method for statistical analysis. For the qRT-PCR data, normal distribution was first assessed, and a non-parametric Mann-Whitney test was performed using GraphPad Prism version 10.0 for Windows, GraphPad Software, San Diego, California USA, www.graphpad.com.

### Cell Proliferation Assays

Cell proliferation assays were performed as previously described (Alhazmi et al., (2020) The promyelocytic leukemia protein isoform PML1 is an oncoprotein and a direct target of the antioxidant sulforaphane (SFN). Biochim Biophys Acta Mol Cell Res 1867, 118707), 1.5-3.0×10³ of HEPACAM2 knockdown H69 cells (Guide 4) or control (pLCP) were seeded onto 96-well plates and allowed to grow for the next 72 hr before assessment with a Cell Counting Kit-8 (CK04; Dojindo Molecular Technologies, USA). OD values at 450 nm were measured using a SpectraMax M2 plate reader.

### Transwell Cell Migration Assay

All the test cell lines (5.0×10⁵ cells/well) were seeded using a transwell insert (8 μm pore size) with 100 μl of FBS-deficient DMEM/F12 and incubated for 10 min at 37° C. FBS-containing media (600 μl) was added to the lower chamber's bottom. The transwell insert was then removed from the 24-well after 48 hr. Cells were collected from the lower chamber and visually counted with a brightfield microscope.

### Cell Attachment Assays

Cell attachment assays were performed following viral transduction (pLCP vs. CRISPR gRNA #4). Briefly, an equal number of control and HEPACAM2 knockdown SCLC cells (H1694) were seeded and permitted to grow for the next 72 hr. The medium was then removed, washed with 1×PBS, and centrifuged before manually counting with a brightfield microscope.

### MMP ELISAS

Spent tissue culture medium from an overnight incubation was concentrated from 100% confluent cells to 10× with an Amicon 30 kD spin concentrator (Sigma UFC803024). Thereafter, the ELISA kit instructions for MMP2 (Invitrogen #KHC3081) and MMP9 (Invitrogen #BMS2016-2) were essentially followed. Briefly, concentrated supernatant or kit standards were incubated in wells that were coated with capture MMP2 or MMP9 antibody for 2 hr, wells washed and incubated with biotin-conjugated detection antibody for 1 hr. After washing, wells were incubated with streptavidin-HRP for 30 min, all at room temperature. Wells were developed and read on a Promega GloMax plate reader. Kit standards were used to construct a standard curve to determine MMP9 or MMP2 concentrations in the concentrated supernatant.

### Visium (10× Genomics, Pleasanton, CA) Spatial Transcriptomic Study

We utilized the VISIUM Spatial sequencing platform to examine the mixed histological subtypes of a combined-SCLC patient tumor and assess their specific gene expression profiles. Since the different histologies of combined-SCLC were very inter-dispersed, each VISIUM capture area contained several cell clusters of each histology. In essence, spatial transcriptomic analyses allowed us to obtain differentially expressed gene profiles of individual clusters of similar (NSCLC vs. NSCLC, SCLC vs. SCLC) or different (NSCLC vs. SCLC) histologic subtypes to identify intratumor heterogeneity in both within and between histologies. The VISIUM technology obtained RNA expression data at a 55 μm resolution across a 6.5×6.5 mm tissue section, and the resulting raw RNA-seq data was then analyzed and visualized using company-supported Space Ranger and Loupe Browser software, respectively. Gene expression, including HEPACAM2, Napsin A Aspartic Peptidase (NAPSA), and Synaptophysin (SYP), was investigated spatially and quantitatively.

### Statistical Analysis

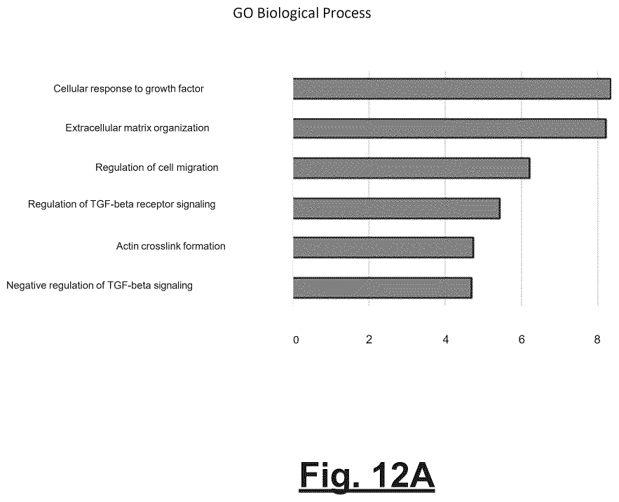

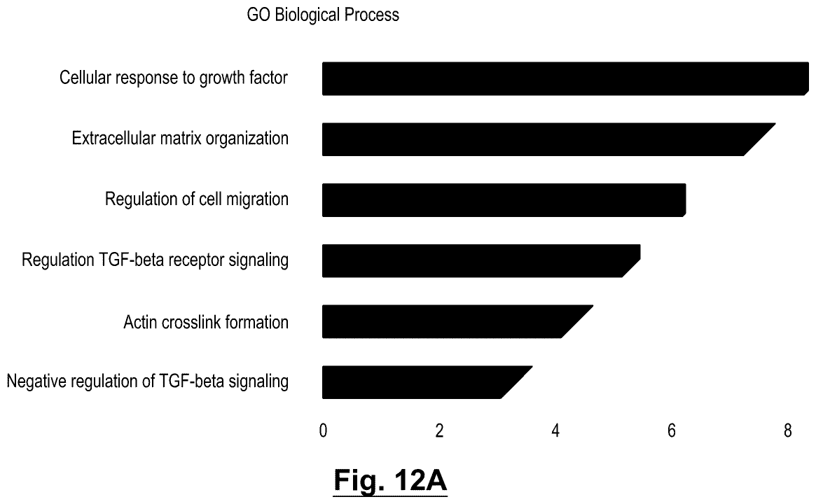

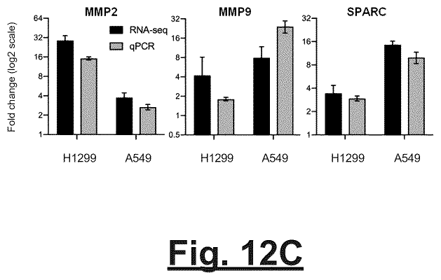

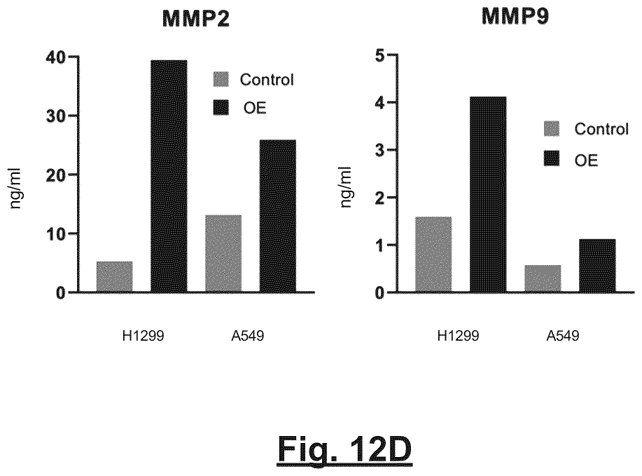

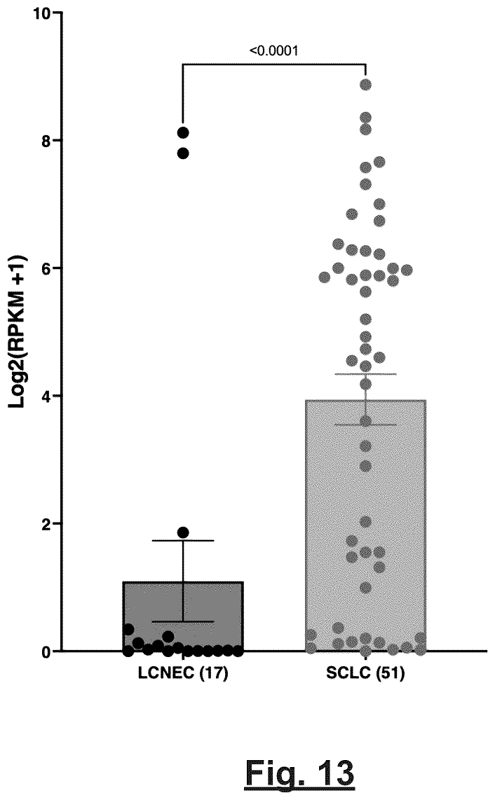

RT-qPCR results represent the mean±standard deviation (SD) of the replicates. RNA-seq analyses were performed on three biological replicates for each cell line per condition (FIGS. 12A-D). Hepacam2-overexpressing tumor growth rate was calculated relative to the tumor volume of the Lv158 (tumor volume on day x/mean tumor volume of control/treated on day 1). All statistical analyses were performed using GraphPad Prism version 10.1.0 for macOS, GraphPad Software, Boston, Massachusetts USA, www.graphpad.com, and R 4.2.2.

### Ethical Approval

All NRG mice (6 wk old; Jackson Lab, Bar Harbor, ME) were kept on 12-hr light/dark cycles in the Animal Resource Core Facility of Case Western Reserve University (CWRU). The Institutional Animal Care and Use Committee of CWRU approved all protocols.

### Results

### HEPACAM2 Expression in SCLC Cells and Tumors

We performed qPCR to validate the mRNA expression levels of HEPACAM2 among a myriad of lung cancer and normal lung cell lines (FIG. 1B). Our results substantiated that SCLC cell lines distinctly express much higher levels of HEPACAM2 compared to NSCLC, and normal lung cells, with the rank order of expression generally paralleling that of the CCLE data. To determine the degree and specificity of HEPACAM2 expression in human SCLC tumors, we compared RNA-seq data from SCLC tumors (Rudin et al., (2012) Comprehensive genomic analysis identifies SOX2 as a frequently amplified gene in small-cell lung cancer. Nat Genet 44, 1111-1116) to that of other solid tumor types in TCGA database. We found that HEPACAM2 mRNA expression is highest in SCLC tumors (FIG. 1C), validating our results from cell lines. Further analyses of RNA-seq datasets (Rudin et al., (2012) Comprehensive genomic analysis identifies SOX2 as a frequently amplified gene in small-cell lung cancer. Nat Genet 44, 1111-1116; and Laddha et al., (2019) Integrative Genomic Characterization Identifies Molecular Subtypes of Lung Carcinoids. Cancer Res 79, 4339-4347) revealed that HEPACAM2 mRNA expression in SCLC tumors was significantly higher than in normal lung, as well as typical and atypical lung carcinoids (FIG. 1D and FIG. 14). The latter are slower-growing, low-grade NE tumors of the lung, which are far less aggressive than SCLC. Thus, only SCLC cell lines and tumors demonstrate consistently high-level expression of HEPACAM2 mRNA.

### Protein Expression and Localization of HEPACAM2

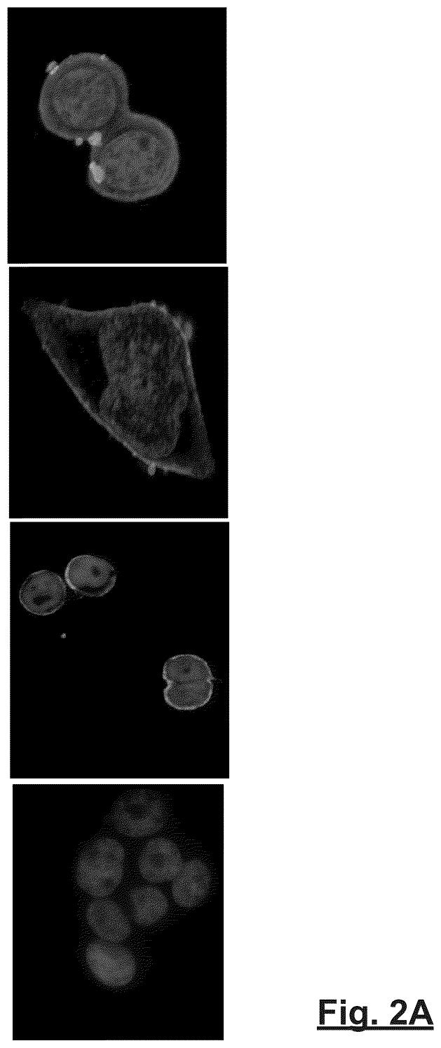

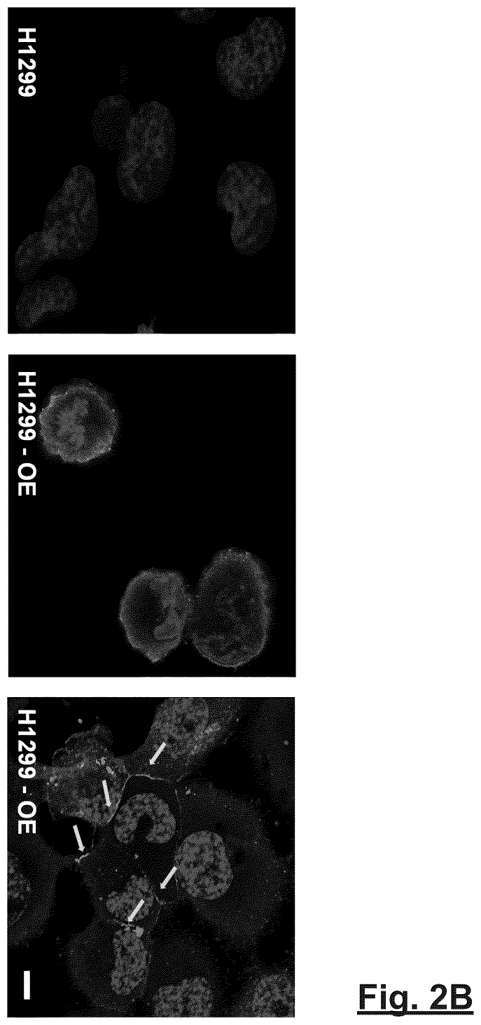

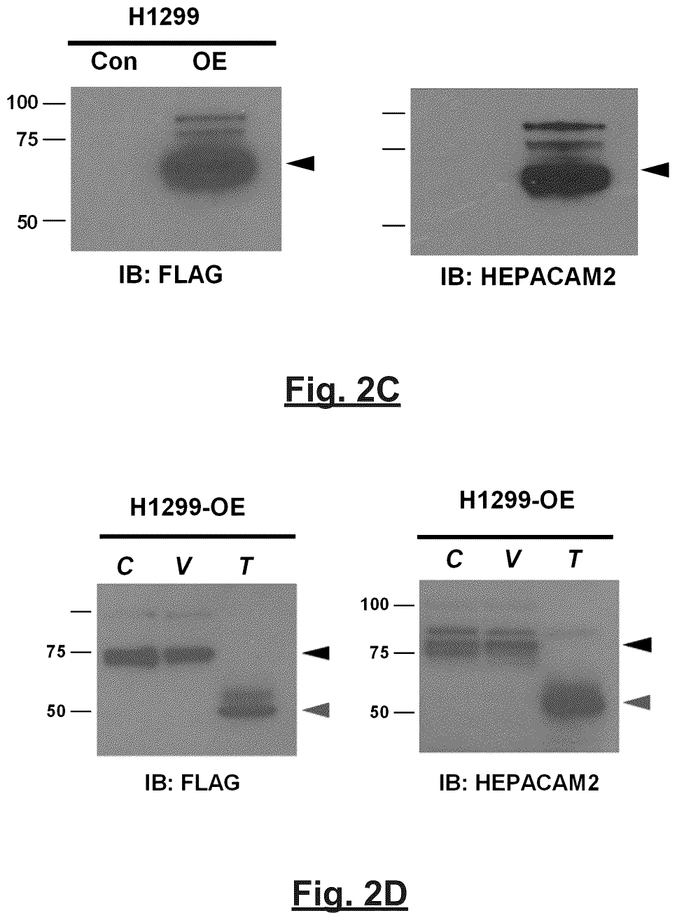

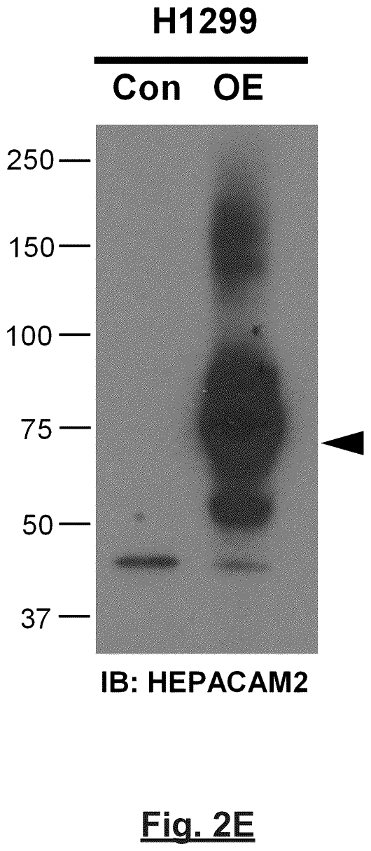

To validate the high and specific expression of HEPACAM2 in SCLC at the protein level, we performed immunofluorescence microscopy on SCLC cell lines (DMS454, H1048, and H2029). Compared to an NSCLC cell line (H1299) with no endogenous HEPACAM2 expression (H1299), all SCLC cell lines express a high endogenous level of HEPACAM2, which appears to be localized to the plasma membrane (FIG. 2A). Moreover, when HEPACAM2 was ectopically expressed in H1299, confocal microscopy confirmed its localization to the plasma membrane (FIG. 2B). We initially observed a ˜70 kDa band in our stably HEPACAM2-overexpressing H1299 cells on western blot, (FIG. 2C), plus two minor bands of higher molecular weight. All these bands, however, migrated more slowly than the predicted HEPACAM2 protein size of ˜50-52 KDa. Since HEPACAM2 is predicted to have six N-linked glycosylation sites, we sought to determine if glycosylation was responsible for the slower migration of HEPACAM2 detected in western blots.

Therefore, we treated HEPACAM2-overexpressed H1299 cells with tunicamycin to disrupt the initial step in glycoprotein synthesis. Using either anti-FLAG or anti-HEPACAM2 antibodies resulted in the appearance of a ˜50 kDa doublet band (FIG. 2D), representing the predicted size for the unmodified HEPACAM2 protein. Finally, to further validate the high confidence with which HEPACAM2 is predicted to be a cell-surface protein (http://wlab.ethz.ch/surfaceome/) and to corroborate our confocal study (FIG. 2B; far right panel), we used a biotin-pulldown technique to determine if HEPACAM2 can be detected on the external cell-surface membrane. With the anti-HEPACAM2 antibodies, the overexpressed protein was detected by this technique (FIG. 2E). Once again, several higher molecular weight bands are also seen, likely representing more fully glycosylated forms of HEPACAM2. Our results suggest that overexpressed HEPACAM2 is primarily a glycosylated protein localized to the plasma membrane.

### HEPACAM2 is Secreted Extracellularly by SCLC Cell Lines

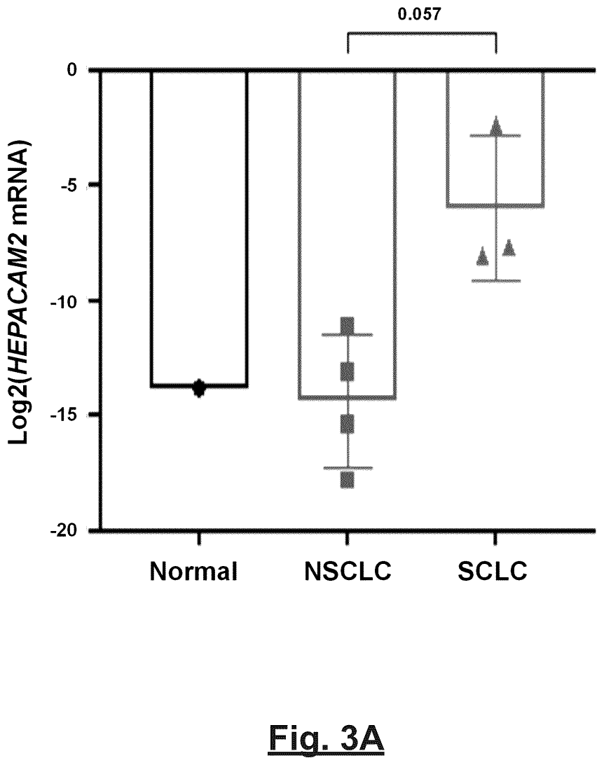

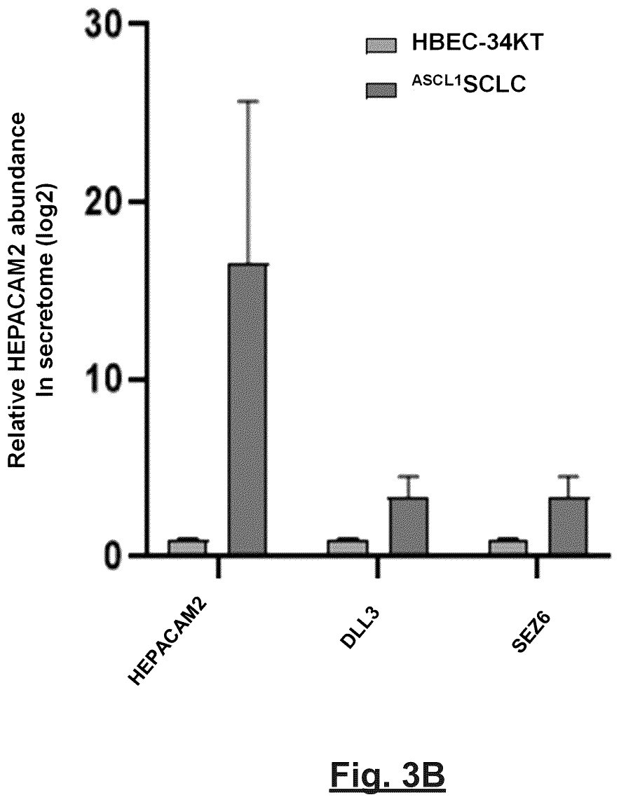

Due to its highly expressed, glycosylated, and cellular localization characteristics, we hypothesized a role for HEPACAM2 in the extracellular space, specifically in SCLC. We thus sought to determine if HEPACAM2 mRNA or protein could be found extracellularly by isolating and interrogating the EVs produced by SCLC cell lines. HEPACAM2 mRNA could be detected at higher levels in the EVs of SCLC cell lines (H526, H209, and H510A) than those from NSCLC cell lines (PC9, H1299, HCC15, and A427) and normal (BEAS2B) cell line (FIG. 3A). Correspondingly, previous analysis of the secretome in SCLC cell line cultures found very high levels of HEPACAM2 as demonstrated by LC-MS/MS, compared to HBEC-34KT, a normal epithelial cell line of the lung (FIG. 3B). In addition, secreted HEPACAM2 protein in these NE-specific SCLC cell lines (ASCLISCLC), is relatively more abundant than some current ADC targets for SCLC, such as DLL3 and SEZ6. Overall, our results confirm that not only are HEPACAM2 mRNA and protein high intracellularly but are also secreted outside the cell.

### HEPACAM2 in Human Small Cell Lung Cancer and Treatment-Emergent Prostatic Small Cell

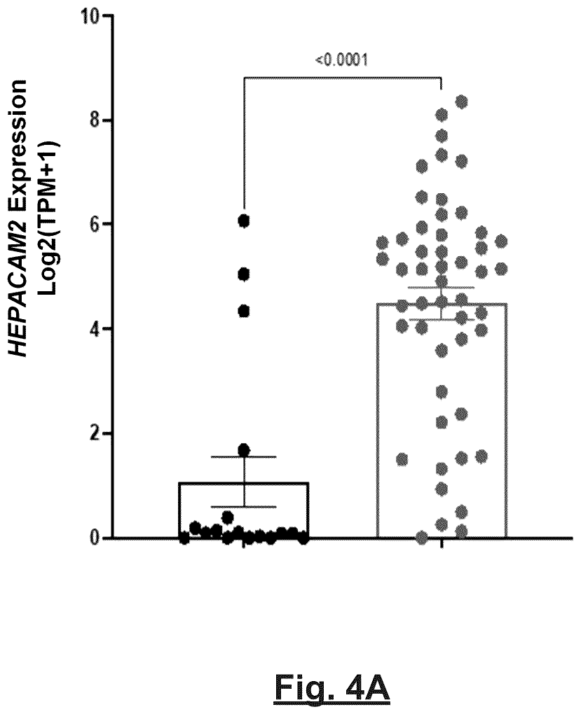

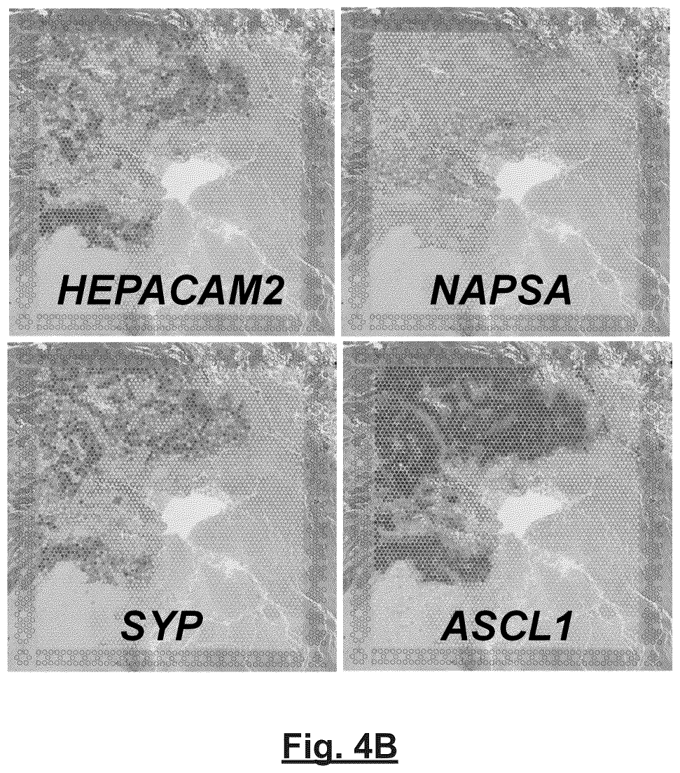

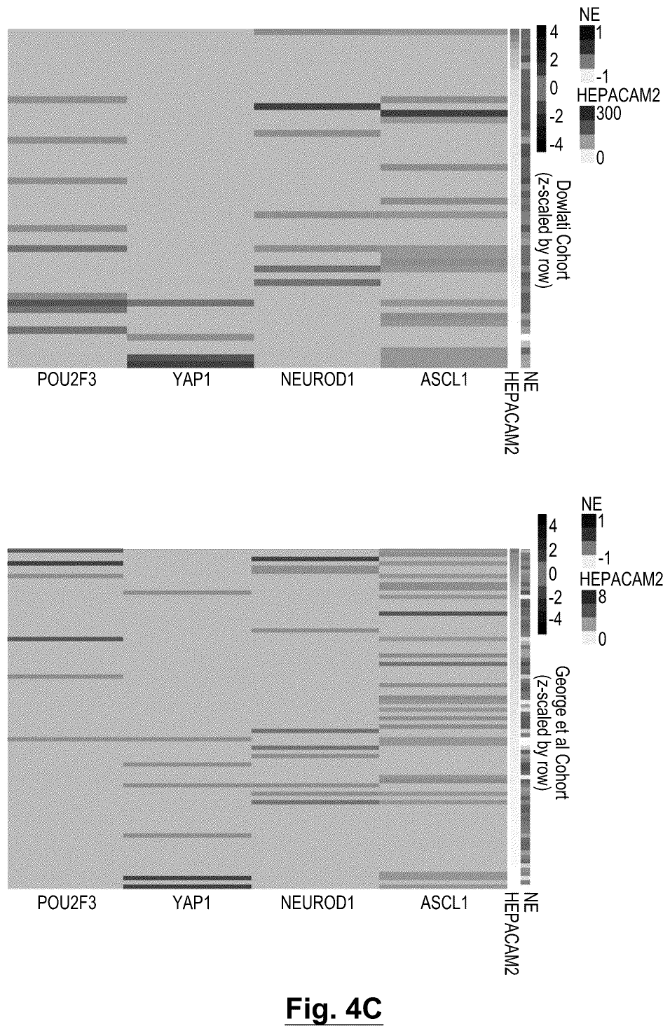

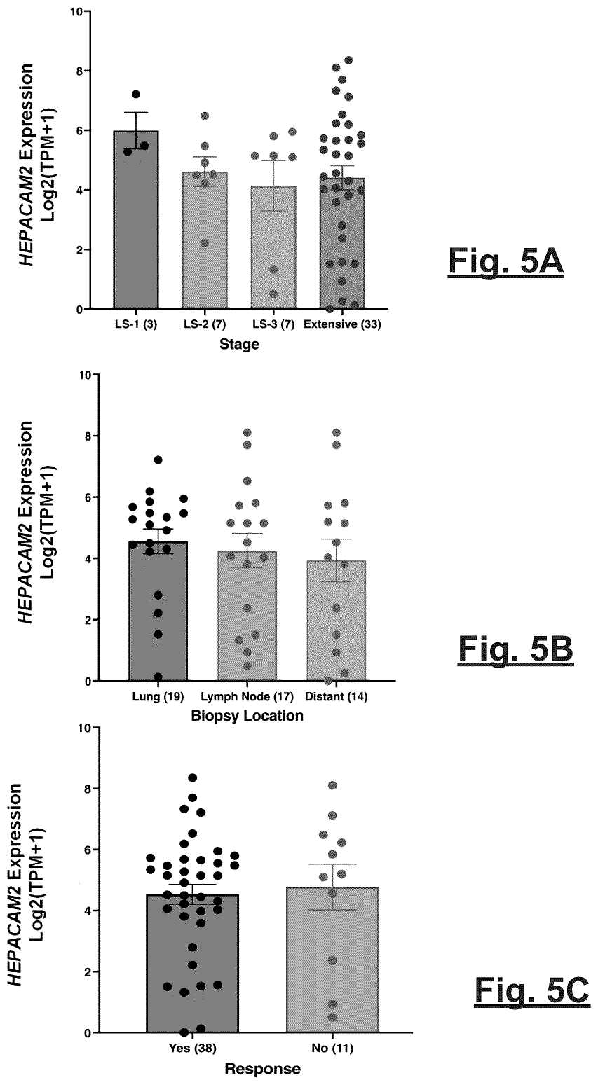

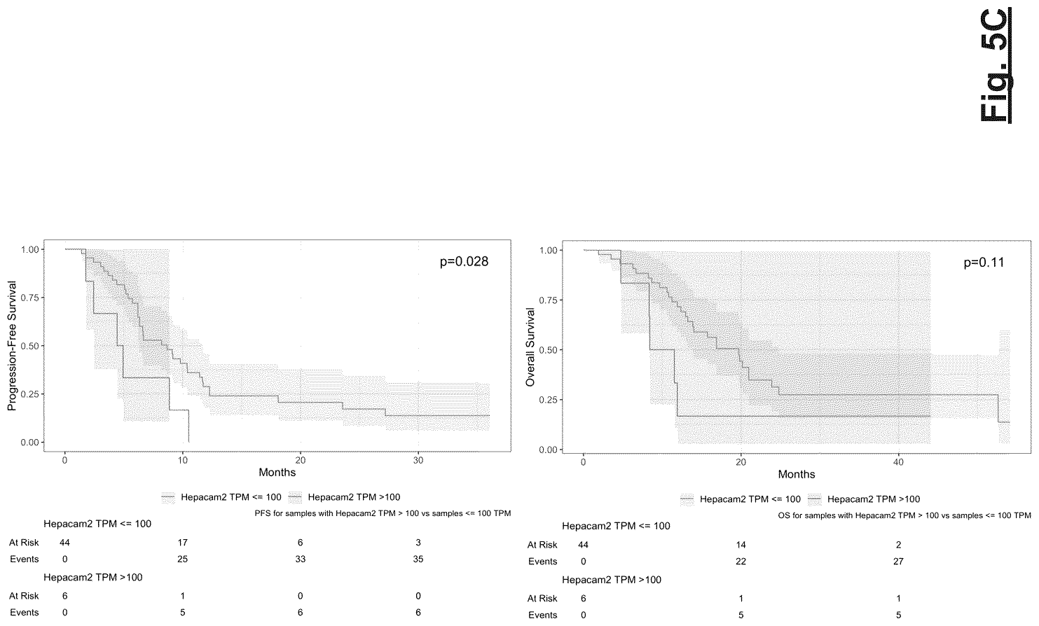

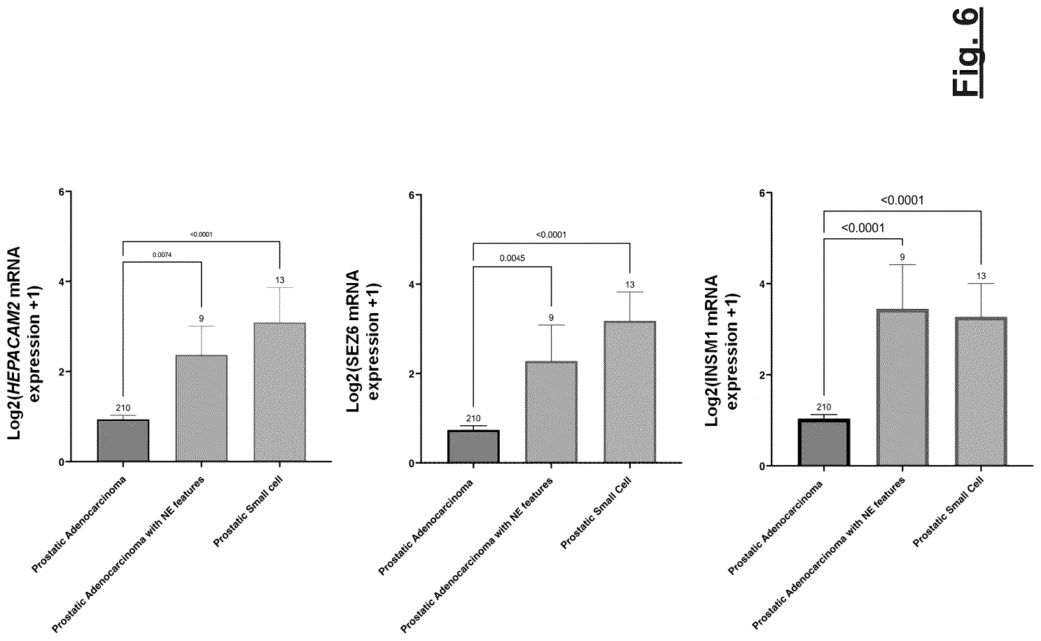

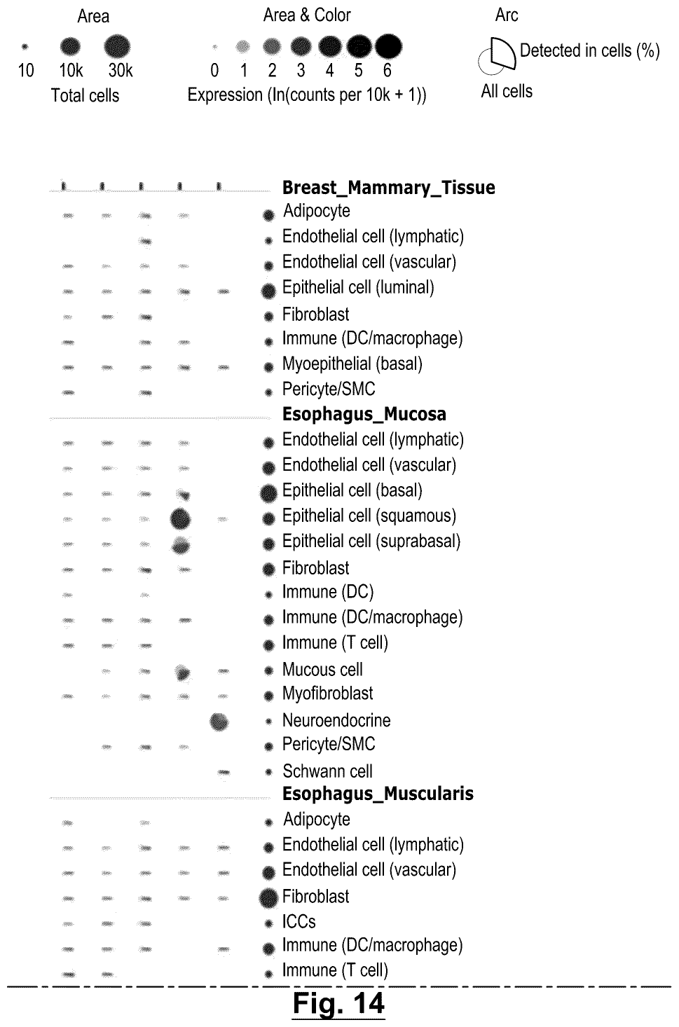

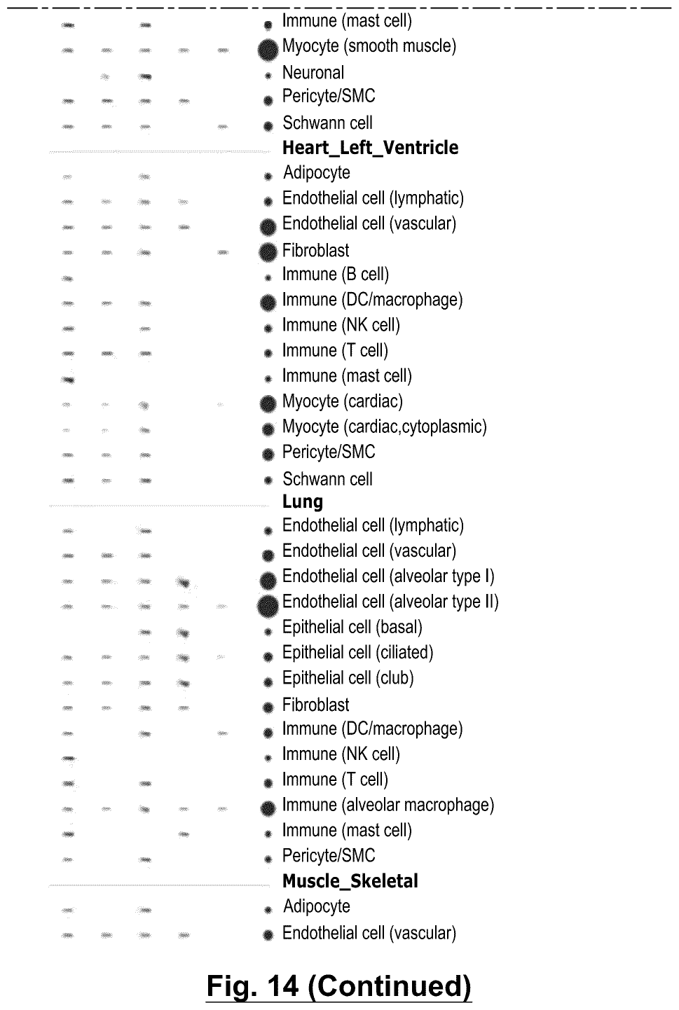

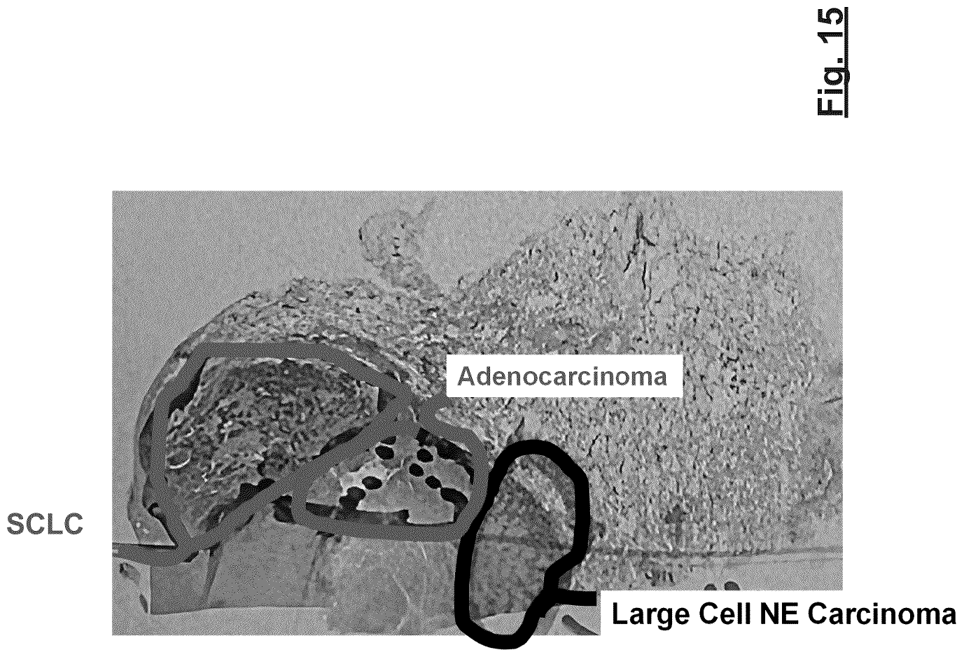

To investigate the potential role HEPACAM2 plays in human SCLC, we used an extensive large collection of our patient samples to confirm further the high level of HEPACAM2 expression by bulk RNA-seq. As expected, our cohort demonstrated very high levels of HEPACAM2 expression in SCLC compared to the NSCLC samples we obtained (FIG. 4A), with a 3.4-fold increase compared to NSCLC patient samples. Moreover, we used spatial transcriptomic analysis of a single case of combined SCLC patient tumor samples to validate the specificity of SCLC. This rare “combined” SCLC biopsy has both small cell and non-small cell elements within the tumor (FIG. 15). The high HEPACAM2 expression is specific to the SCLC region and overlaps with the NE markers of SYP and ASCL1. However, it is not seen in the NSCLC area, i.e., high NAPSA-expressed region of adenocarcinoma (FIG. 4B). Furthermore, heatmaps generated for RNA-seq datasets from two different SCLC cohorts indicate that HEPACAM2 expression correlated with the NE subtype of SCLC and is associated more commonly with ASCL1; rather than NeuroD1, POU2F3, and YAP1 (FIG. 4C). Evaluating HEPACAM2 levels in various stages of SCLC, we found no difference comparing stages 1, 2, 3 (limited stages) and stage 4 (extensive stage SCLC), suggesting that HEPACAM2 expression occurs in an early-stage SCLC, not just in the metastatic disease in our cohort (FIG. 5A). This feature may have importance in early diagnosis strategies as HEPACAM2 elevation is seen in earlier as well as later stages of SCLC. In addition, we evaluated HEPACAM2 expressions in various SCLC biopsy sites. We found that there was no difference in where the tissue originated from, indicative of its persistence in SCLC tumors in even metastatic tissue (FIG. 5B). We did not observe a difference in response to chemotherapy in patients with SCLC based on HEPACAM2 expression levels (FIG. 5C); however, we found a diminished PFS and OS in patients with high HEPACAM2 expression (FIG. 5D) when we stratified these patients into a well-defined high (n=6) vs. low (n=44) HEPACAM2 levels within the same cohort (FIG. 16). Finally, we investigated whether HEPACAM2 expression was specific to SCLC or could be seen in small cells of extrapulmonary origin by analyzing RNA-seq datasets (Abida et al., (2019) Genomic correlates of clinical outcome in advanced prostate cancer. Proc Natl Acad Sci USA 116, 11428-11436) from 3 groups of prostate cancer patients, which included adenocarcinoma, adenocarcinoma with NE features, and prostatic small cell carcinoma. HEPACAM2 expression rose progressively between the three subtypes, suggesting that as adenocarcinoma of the prostate transforms into a more NE phenotype, HEPACAM2 expression increases (FIG. 6).

High HEPACAM2 Expression is not Synonymous with Other Current Targets of Antibody-Drug Conjugate Therapy

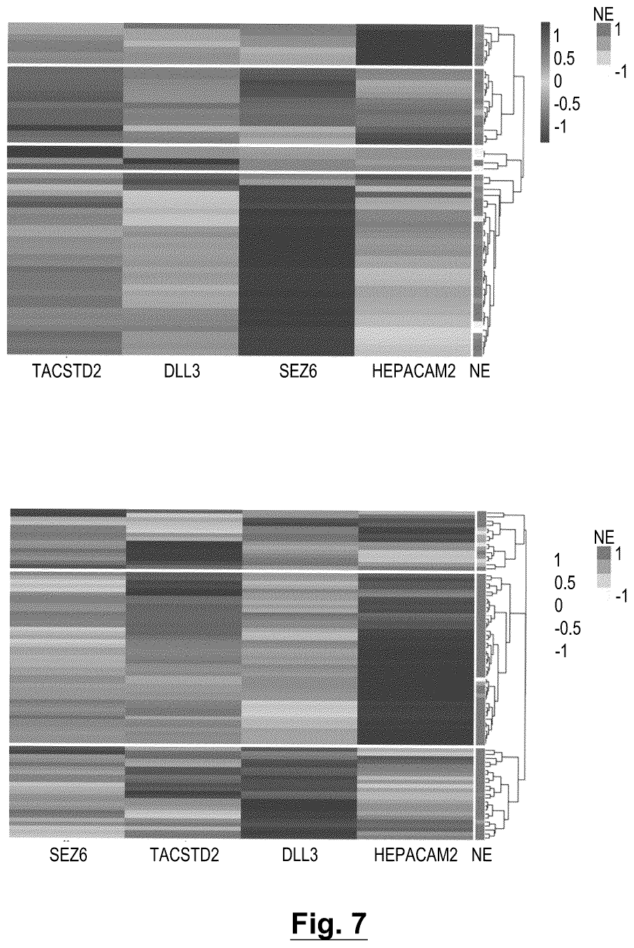

HEPACAM2, with its high expression and specificity for the cell surface of SCLC cells, might help to expand and improve the therapeutic options presented by antibody-drug conjugates (ADC). As such, we further analyzed the available RNA-seq datasets (George et al., (2015) Comprehensive genomic profiles of small cell lung cancer. Nature 524, 47-53) and assessed how HEPACAM2 compares to existing or proposed targets for SCLC ADC therapy, such as DLL3, SEZ6, or TROP2 (aka, TACSTD2 gene). Importantly, we demonstrated that high HEPACAM2 expression only partially overlaps with other ADC targets and identifies a discrete cohort of SCLC patients that are not marked by high expression of alternative targets (FIG. 7), making it a potential and specific therapeutic target, resulting in less potential toxicities from non-target tissue binding.

### Functional Effects of High HEPACAM2 Expression

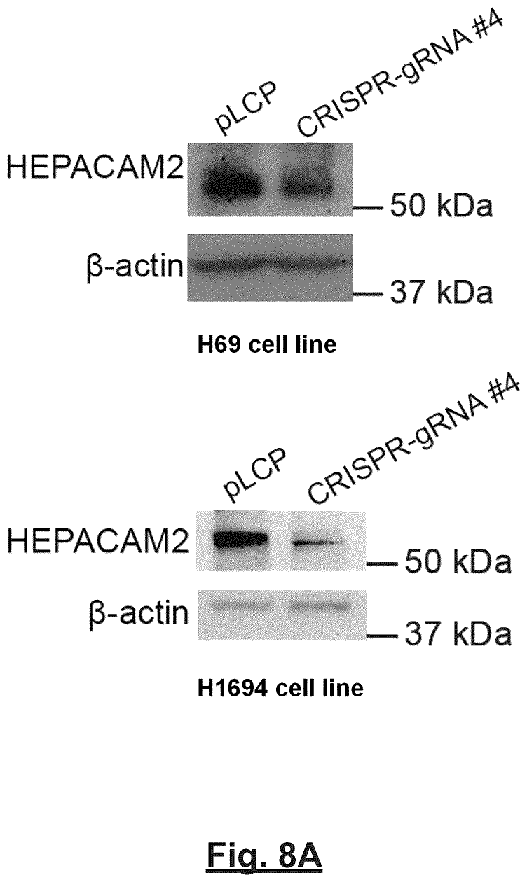

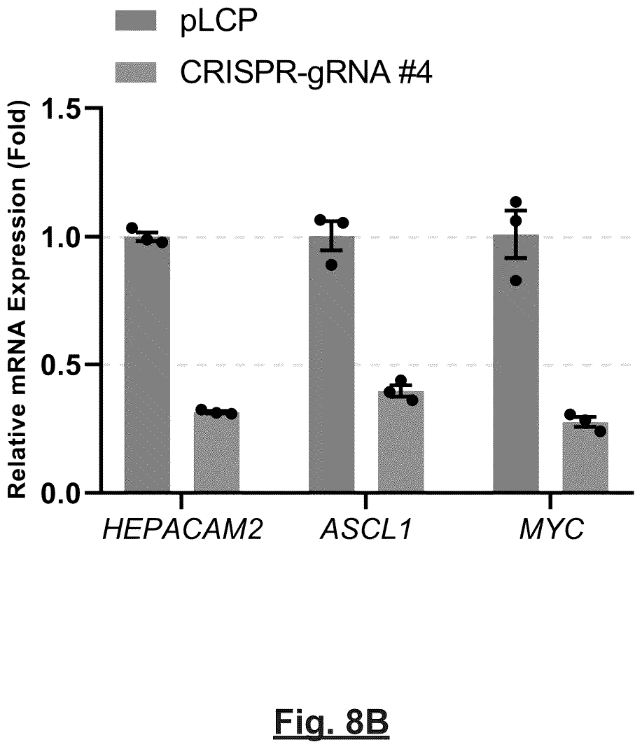

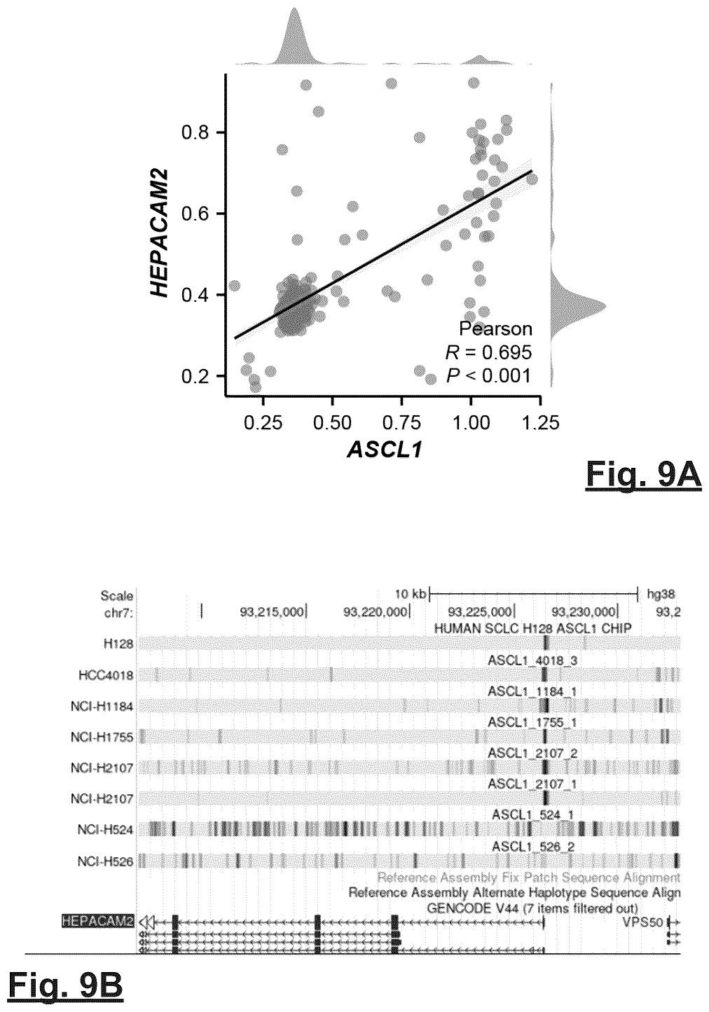

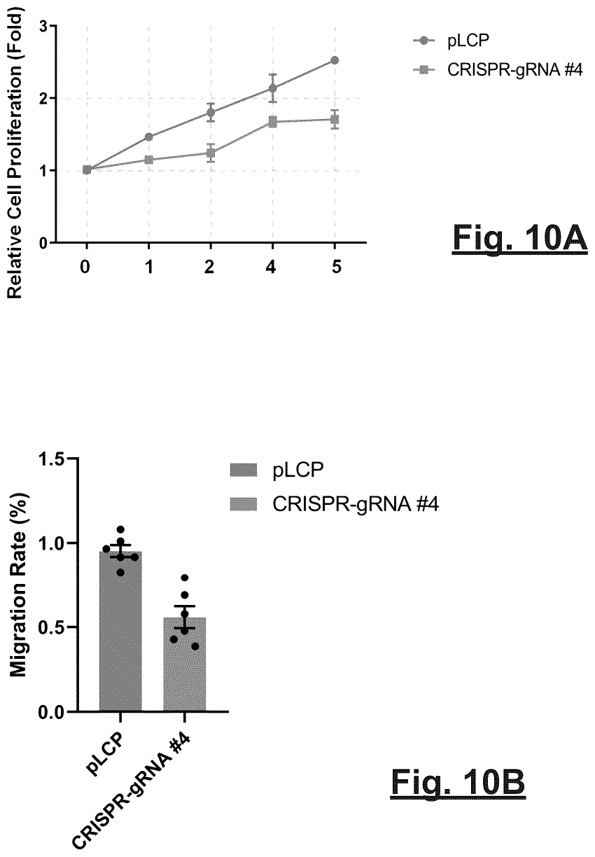

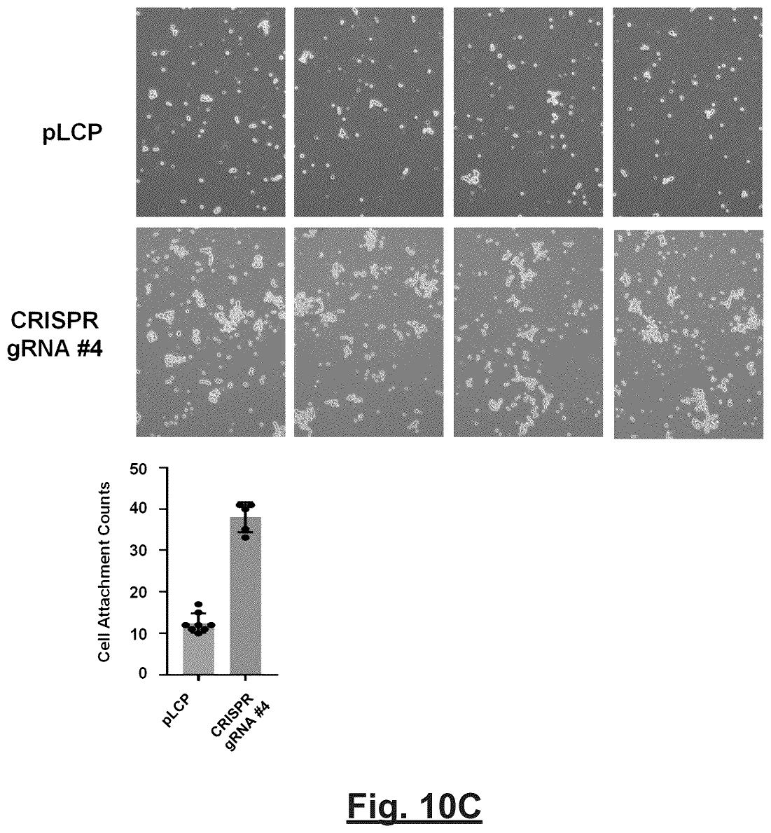

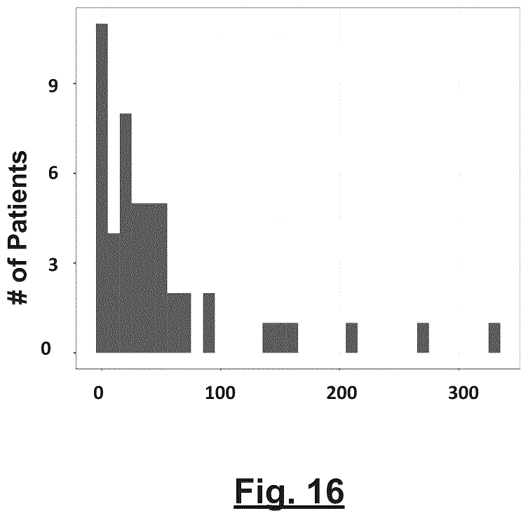

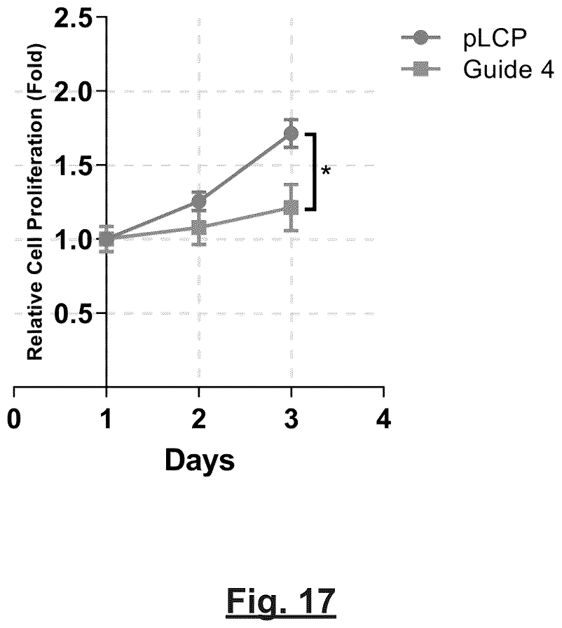

To establish HEPACAM2 as an ideal target for antibody-drug conjugate therapy, in addition to its high expression levels, cell surface localization, and specificity in SCLC, understanding the biology of HEPACAM2 is important. Therefore, we examined the functional effects of the HEPACAM2 expression in vitro by examining two SCLC cell lines expressing endogenous HEPACAM2. Western blots revealed a significant HEPACAM2 knockdown in H69 and H1694 cells (FIG. 8A). In these knockdown cell lines, we also observed a substantial reduction in mRNA levels of ASCL1 and MYC (FIG. 8B), suggesting a potential link to the more aggressive nature of cells with higher HEPACAM2 expression. Moreover, the level of ASCL1 is positively and associated with HEPACAM2 expression (FIG. 9A) with a Pearson Correlation Coefficient of 0.695, leading us to postulate whether ASCL1 regulates HEPACAM2 or vice versa. Consistently, the ENCODE database indicated that ASCL1 has a putative binding site to the HEPACAM2 promoter in several SCLC cell lines, suggesting that ASCL1 regulates HEPACAM2 expression (FIG. 9B). This data aligns with the patient data, which reveals a positive correlation between ASCL1 and HEPACAM2 mRNA expression (FIG. 4B). Notably, the knockdown of HEPACAM2 also significantly reduces ASCL1 mRNA expression (FIG. 8B). Thus, our data may have uncovered a positive feedback loop between ASCL1 and HEPACAM2. With lower ASCL1 expression in HEPACAM2 knockdown, we next investigated whether decreased HEPACAM2 expression can lead to phenotypic alterations in these SCLC cells. We found that knocking down HEPACAM2 decreased cell proliferation (FIG. 10A and FIG. 17) and migration significantly (FIG. 10B). Furthermore, HEPACAM2-knockdown SCLC cells displayed increased attachment to the plates, a behavior similar to NSCLC (FIG. 10C) cells. These findings suggest that high HEPACAM2 levels in these SCLC cells are associated with NE characteristics, as evidenced by the significant increase in cell migration and proliferation.

To correlate our HEPACAM2 knockdown experiments (FIGS. 10A-C and FIG. 17), we examined how HEPACAM2 expression may lead to changes in cell-matrix and cell-cell interactions, we performed RNA-seq analyses and identified common features in the genes similarly regulated by HEPACAM2 overexpression in A549 and H1299 cells. Interestingly, overexpressing HEPACAM2 in NSCLC cell lines did not affect ASCL1 expression. This analysis revealed recurrent hits in genes associated with extracellular matrix organization, focal adhesion, and cytoskeletal organization (FIG. 12A). These findings were more evident when the gene datasets were thresholded to include only those genes commonly changed by at least two-fold, which caused a 20-fold reduction in shared genes and was biased towards up-regulated genes (FIG. 12B). Specifically, ectopic expression of HEPACAMP2 elevated the levels of MMP2, MMP9, and SPARC mRNAs. This analysis was further validated by qPCR (FIG. 12C) and ELISA (FIG. 12D).

### Additional Results

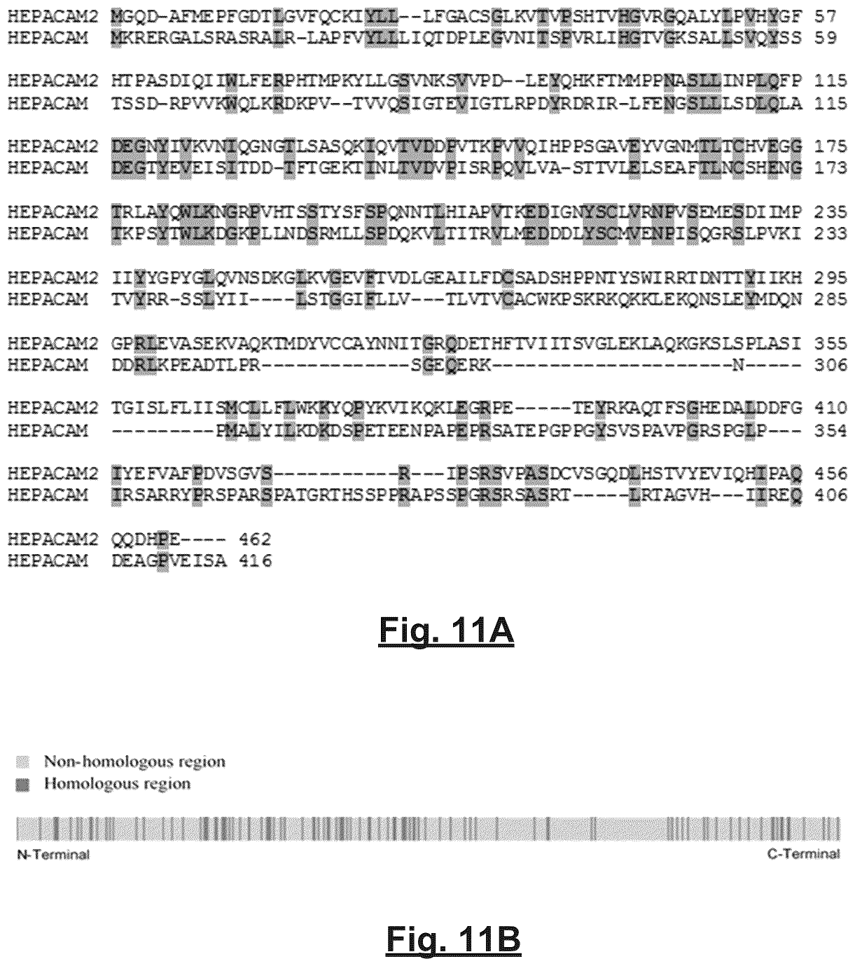

HEPACAM2 shares only 22% protein homology with its related Ig superfamily member, HEPACAM (FIGS. 11A-B).

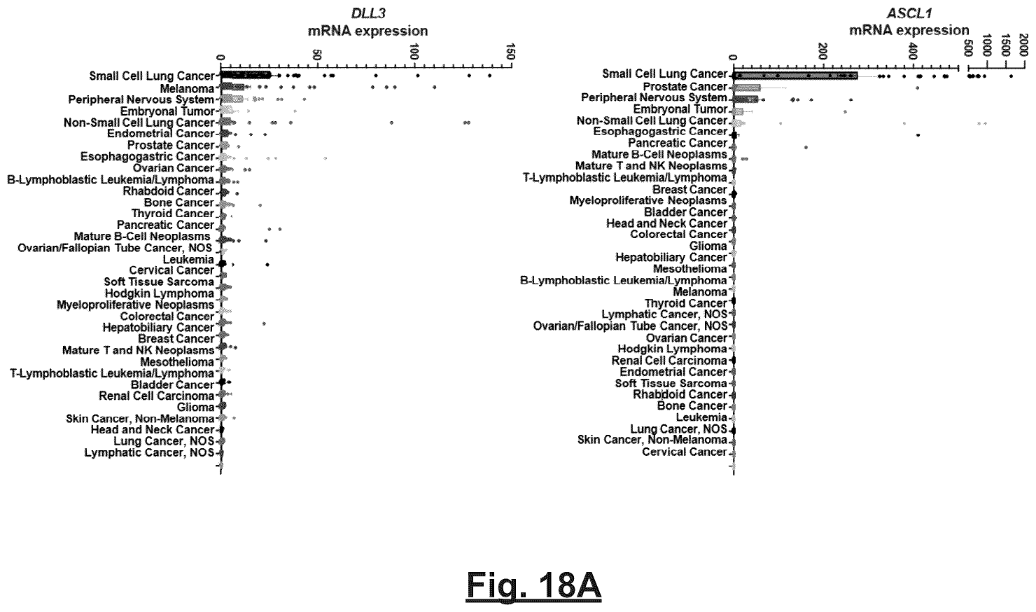

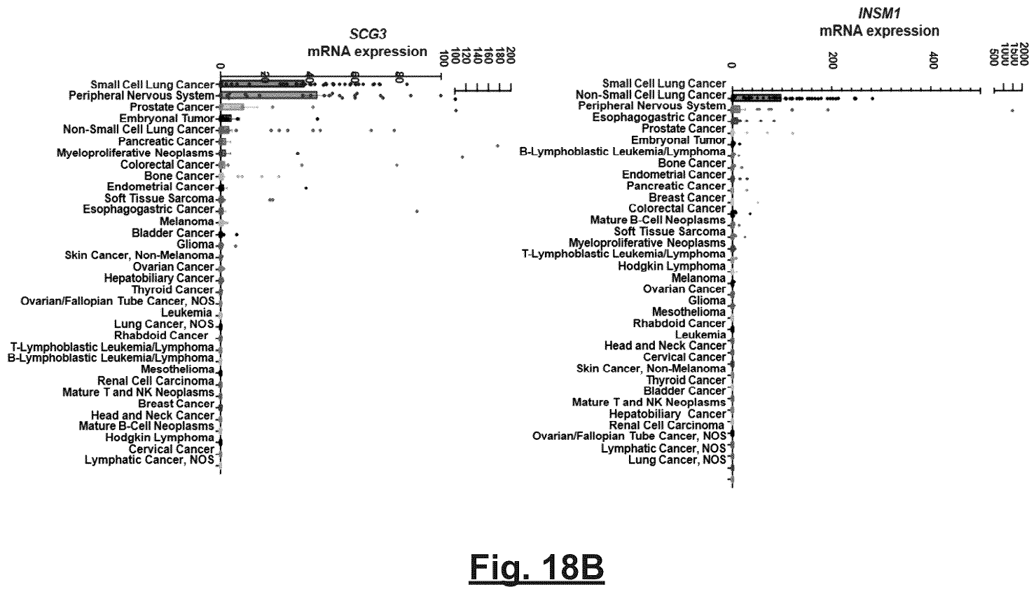

Here, for the first time, we identify HEPACAM2 as a gene that is specifically and highly expressed in SCLC relative to all other cancers. The specificity of HEPACAM2 for SCLC compared to all other cancers in the CCLE is greater than that of the three NE genes (ASCL1, INSM1, and SCG3) and DLL3 (FIGS. 18A-B), which demonstrated more significant enrichment in SCLC versus NSCLC in our initial screen.

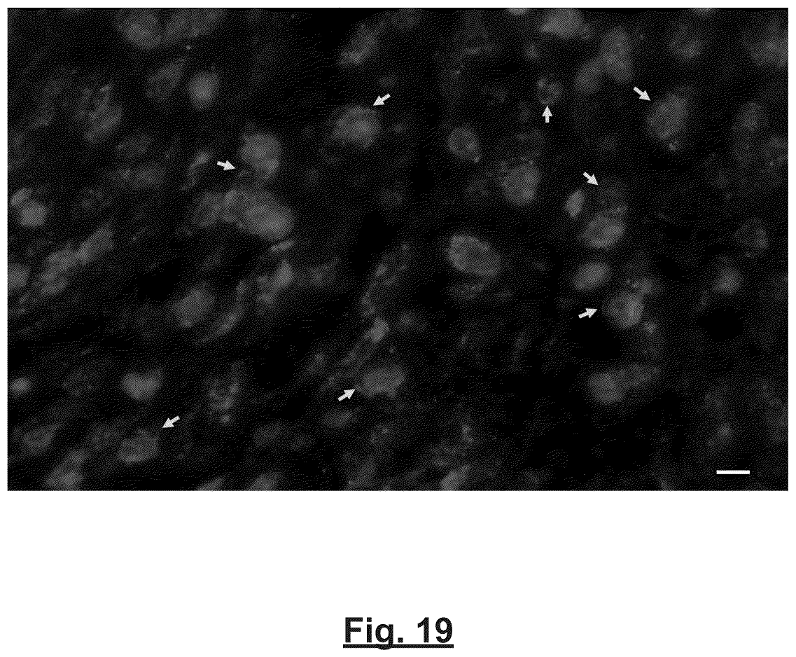

We also validated that HEPACAM2 is glycosylated and localized to the cell surface, in agreement with bioinformatics predictions based on its primary structure, as well as being a consistent feature of most other members of the Ig superfamily. Immunofluorescence staining revealed endogenous HEPACAM2 localizing to the plasma membrane in numerous SCLC cell lines as well as an NSCLC cell line ectopically expressing HEPACAM2 (FIGS. 2A-B and FIG. 19). The high expression, specificity, and cell surface localization of HEPACAM2 in SCLC may make it an ideal target for antibody-drug conjugate (ADC) or chimeric antigen receptor T therapy.

From the above description of the present disclosure, those skilled in the art will perceive improvements, changes and modifications. Such improvements, changes, and modifications are within the skill of those in the art and are intended to be covered by the appended claims. All patents, patent applications, and publications cited herein are incorporated by reference in their entirety.

The following is claimed:

1. A method of diagnosing a patient with a neuroendocrine carcinoma, comprising assaying for an expression level of a hepatocyte cell adhesion molecule 2 (HEPACAM2) biomarker in a biological sample previously obtained from the patient, wherein a detected expression level of the HEPACAM2 biomarker that is higher than a control level of a HEPACAM2 biomarker is indicative of a neuroendocrine carcinoma in the patient.

2. The method of claim 1, wherein the neuroendocrine carcinoma is small cell lung carcinoma (SCLC).

3. The method of claim 2, wherein the SCLC is early-stage SCLC.

4. The method of claim 1, wherein the patient is a member of a high-risk population for SCLC and is free of metastases.

5. The method of claim 3, wherein the patient does not exhibit a high expression level of DLL3, SEZ6 and/or TROP2 as compared to a control expression level of DLL3, SEZ6 and/or TROP2.

6. The method of claim 1, wherein the expression level of a HEPACAM2 biomarker is expression of a HEPACAM2 mRNA and the expression level of a HEPACAM2 biomarker is a HEPACAM2 protein.

7. The method of claim 6, wherein the HEPACAM2 protein is expressed and localized at a surface of a neuroendocrine carcinoma cell.

8. The method of claim 1, wherein the assay is performed on one or more extravesicles secreted by a cell comprising a neuroendocrine carcinoma cell or a cell suspected of comprising a neuroendocrine carcinoma cell.

9. The method of claim 6, wherein qPCR is used to assay the expression level of the HEPACAM2 mRNA and immunofluorescence microscopy or a biotin-pulldown technique is used to assay the expression level of the HEPACAM2 protein.

10. The method of claim 1, further comprising administering a therapeutically effective amount of a therapeutic regimen to a patient diagnosed with a neuroendocrine carcinoma, the therapeutic regimen being a therapy selected from the group consisting of chemotherapy, targeted therapy, radiation therapy, immunotherapy, surgery, and combinations thereof.

11. The method claim 10, wherein the therapeutic regimen is administered subsequent to the beginning of a relapse episode.

12. A method of determining a survival prognosis in a patient diagnosed with a neuroendocrine carcinoma, the method comprising:
assaying an expression level of a hepatocyte cell adhesion molecule 2 (HEPACAM2) biomarker in a biological sample previously obtained from the patient; and
calculating a survival prognosis, based on the assayed expression level of the HEPACAM2 biomarker, comprising an overall survival (OS) and/or a progression-free survival (PFS);
wherein a detected expression level of the HEPACAM2 biomarker that is higher than a control level of a HEPACAM2 biomarker is indicative of a lesser OS and/or a lesser PFS as compared to a control patient.

13. The method of claim 12, wherein the neuroendocrine carcinoma is small cell lung carcinoma (SCLC) and the patient is diagnosed with Stage 0, Stage I, Stage II, Stage III, or Stage IV SCLC, based on the TNM staging system.

14. A method of treating a patient having, or suspected of having, a neuroendocrine carcinoma, the method comprising administering a therapeutically effective amount of a therapeutic regimen to the patient, wherein the neuroendocrine carcinoma is characterized by an expression level of a hepatocyte cell adhesion molecule 2 (HEPACAM2) biomarker that is higher than a control level of a HEPACAM2 biomarker.

15. The method of claim 14, wherein the therapeutic regimen is a therapy selected from the group consisting of chemotherapy, targeted therapy, radiation therapy, immunotherapy, surgery, and combinations thereof.

16. The method of claim 15, wherein the targeted therapy comprises an antibody-drug conjugate or a RNA interfering modality.

17. The method of claim 15, wherein the immunotherapy comprises a CAR-T cell therapy.

18. The method claim 14, wherein the therapeutic regimen is administered subsequent to the beginning of a relapse episode.

19. The method of claim 14, wherein the neuroendocrine carcinoma is small cell lung carcinoma (SCLC) and the patient is diagnosed with Stage 0, Stage I, Stage II, Stage III, or Stage IV SCLC, based on the TNM staging system.


SEQUENCE LISTING (9 sequences)

SEQ ID NO: 1 - DNA, 22 bp, synthetic construct
  source 1..22: mol_type = other DNA
ctctacctacccgtccactatg

SEQ ID NO: 2 - DNA, 21 bp, synthetic construct
  source 1..21: mol_type = other DNA
tgtggggtctctcaaatagcc

SEQ ID NO: 3 - DNA, 19 bp, synthetic construct
  source 1..19: mol_type = other DNA
cgcggccaacaagaagatg

SEQ ID NO: 4 - DNA, 20 bp, synthetic construct
  source 1..20: mol_type = other DNA
cgacgagtaggatgagaccg

SEQ ID NO: 5 - DNA, 19 bp, synthetic construct
  source 1..19: mol_type = other DNA
ggtgctccatgaggagaca

SEQ ID NO: 6 - DNA, 20 bp, synthetic construct
  source 1..20: mol_type = other DNA
cctgcctcttttccacagaa

SEQ ID NO: 7 - DNA, 37936 bp, Homo sapiens
  source 1..37936: mol_type = genomic DNA
actgagaacagggcactgcatgcatgggacaggatgctttcatggagcccttcggtgacacacttggggtctttcagtgcaaaatatacctccttctcttcggtaaggccctgtgtgaattgtgttttagctgtttgttaggttttttttttttaattgtggacataaggtaaatatttgctttaggctgtagatgcattcagataattgaagagcaggtcagaaccttttaacaataacacactgttacagcaaagaagcaggaacatttaagagaaatgagtttcaataaatagattgttattgttttcttggaacaacaattattgtcatagcactcattaagataattcagatatttttaaaagataatgaatttcttcttttagagtttacatataggttcaatagtctttaatgcatttatttcagcaaatattctaataggccatgaaattgtgaccacttagagtgaaactacaaataatttacgaaaattgttttctttttaattaaagatattgttgcaaactgtttactgcttcgttttaagttatctgtctcatcttatgaaatatggaagaaaggtaagattccaaactaacaaaaaaatttcctacacaaaagggagaacgtatttgaaaagattgttactccaaaatgcctttagcttttctctttgcccaatggctagtcctattaagaatttttttctaccttaatattttccctcattctcctctttatcttttgagtttaaaggagtctctttccagcattccaactgctgtctgtcaacattattttcaccagatgttctcatttccattcttcctatttatcagttttaaaagtcaaatcaatttcttgttttgtttatattcaacagataggagttcaattaaggctacattttttaaaaattaagaacagcttagcaaaataagcaatttaacttttaggcttcttcagctatttagagctttactttcactcctatattttgttaaagtttgcattgtaaatgttattattcaaattcatacctactgttgtacctatctcacacatttgaaatgtacagtttgtaaatttttttaagtctttgagtttcccagagttgtgattgttccagttatcacagtatttatgttatcaaatttaagcctataatagatttttgttttctaaaattttttgaaaaagtaaataattattataactgtcactacctttgtgcatttgtcttccttttgaaagtatcctgggtcatcatcccacttatttttatatttaaaacagtaggactactatcattttataagtgttgtttttgaaactggcgttaagaagaacttagagacaatttagttaaggaattcaactaaaataaaacaaatgtacattaggaaaatagcatgttcataaatagctaaagcagtttttataccttaagaccttctctcttctgcaaatttgcttttctccataggcagtgtgttgtaaagaccacagatgacttggcaatctttgcttttaggaatattgaagttttgtttgcttcttctttttaaaaaatttttaaaaatttgaagcctaaaatgtacagtttgtacagtttattcaaaacacatcataatttctattttctctaaagatgcaagattttattttcagtcaacaatacttgtgttatgcttgttaataccacacatgataacagggaagtaaatgccagctattttgagctacagcagaggtcactttgtgaaatctgatgtttatattcaatacaatgaaaccggtctagggagtcctttgttgctggaatatagataaattctacagtaggacttcaagtgtaccttaacactggatacttttttatagtgctattgtcatacaatgtaagagggtctgtaaaggagggatcattgctgcaattttgtatgtcagattatgattagtattataaatgtttctccagatttgtgtaatgccttatatgcaaaaagtcaagatttttttaacagaactataatttataagagtttttgtttgttatactcagaaagtaatagtgaatataataataaaatcgaatttaaatttccgtttaagtccaaatatttggactactaaaaataaatttaaatttcatcaatttttatattatttattatttttgtttttgtattttttcaaatagagacagaggtctcgctgtgttgcccaagctggtcttaaactcctgggctcaagcaatctgccacctcagtcccctaaagtgcctgggattacaggcattagccaccacaaccaacctttttatattatttattttcttgaagtttatctgttcatgcagaggatgattttctaaaatcaataggtttagtcctttgggttttctcatacaactagaaaagagcacttgaaaatttctcaaaatgaagctaagttttctcacattagtaaactactgtgtttgacagcatgaattcatcacatctgtagtgggtgacacatcaatgataagagagtatgtgttcctagaatgtggaggaagcattcaattatctgtttattcccaaatttcactacaagcaaagattgtatcatattgataggttcatgtccaatgaagagattttacgtggctgagataagatactgtataaagaaaatttcattccctgctgacattcttataacttgctacctataatttatctgataaaaaattgtataggcaatctctaagcaaatagttactgtaacagctgtgctatttctcactgtcacacaaacatttgaaaaagcatacatatttgaaaaaaacttttcatttaataagaaagcactgaacctattcagagatattgatggcaatatattatttaaggtattattacatgatgcagtctaaaatagaccccaaaactaattttcccctaaataatgatcacaaacgctatattaaatatctttttctactaatgtagtgttattttttctcagagcttcaatatttgtattgtatttaatatttcattaaataaaattttcctcctataattttagtcatggactctctaaccagtcttttcatatttaagattcaacattctgggtcaagatccaaaagtcatcaacatttctattccagcaatattgtcaccccatagtaatatagtgcctcccaaccatttccagaatattaaataaatctcaatcttttcaaatataactacaaaggaagtcaattacaaaaaaagcagagaaggacttttgcaatggctttcccaaatgacaagcacaaaatgggtcagaatccattcagaccttctcagcctaatggtggtttctaggctgagttgtgaaatgtcctaagtgaaccaacacatccatgacaaagctgctccctattagcagccatgcagctttttccagtgcattcccaaggcaattatacagacttccatcattccccttgatttctttgtaaaaaacacagaattgctgttttattaaaggatatccaaggattgacaacttaatatatgaaccaaaaactgggactcaattcttcaaaattatttaggccctctctcaatttgggtcctctcaaaactctctgggcccttcaccttcaaatataggtagctgctacccttagaaaagcacctcaggttgtcaataacataaaattaagagagtcgagaaaggccttacaccttatctgatattacctcctattcacacatgagaaaactgagtctgaaaattaaaatgacttatttaaggtcacataactgaccaatcaccattttaccccaagtatgtgtcctttactaggacttcatatttcagtagatctttaactttattaaagtaacaagcacagtaatcttcaagttaagagaataggtgcttgaaaatcattattttagagctatttgcaagtgataaatttaagactcaatggcggacatagaacatgggagtccaagatcaggagtgagccatgaaacacttgtaaatattttcatatttacacaagtaaaaaaaaaggagggagcaatacacttaatacagtgaaaggtccaatttacatatcttaagttagtaataacaggtactaagcctatttttttttgttgtcgcttaccaggaacttgtcaggaactgcctatcttcaacttcaatgaaagacatataaatgcattttgttttgtacagaaaacagatagacaaagaggaatcttttcctgtgctgatctcccatccaaaggcccaatgtcttactaaacctatgactttaagagtttcaccccgactgtcagcctctgcttcacatatcttagacatcactaattttgagacatagatttctctctcccatctttctttctctctcctactatttccttgcgaaaaccactacttcttttctgcagtccactttatctcctctttagaattcccagcacccagttttctctttttctaatcactcaaattaggaaaaattccagatttaatagtcttacacttggcagaaaagaatacagaagggaattctcatctttgtaactgctttgttatggaccaatcaccaaagtgtgcaagtaggtccattatcagaagaaaaaaaaattcttagaagttaatcacaaaaacacacagaggccaatgagacaataagacatgtctttatgtttcttttacagtcatctgcttttcagtccatatggtaagtttcagttgcaagtctaggagtctcttctccctctaactaactatcttcctctttggtgttttctcctcttggattctttcctctaacttctctgccaaccccagagtctcactctctactaatagctagccaaaggcccaaactgtgggaatctttgcttctacctccactctgggaaggaaggagataaatttactgttggtagaactggattcctcataggaacaggcctacatctttgagtttttccaaggaaataatcacaaaaggcaaacagatctttctacctttccctttttaaaattgatggcctttcatgacatgacttggatgaagcttgaggactccctttctagtagaatgaaatcaaaaaaatgtagaaaaaaatgaaaaagtggaagcgatcaaagaggatgaacattttgtacttgagaaatttaaaattgtaaaatgcttctatttatatctaatttccatctcttctcacctgcttaaggattttattccactttcacccatctctattctgcctcatcaacatttccctttccagtgaaacattcccaatagcaaaaaactctctttatttcacatttttgatccttctccagcttctgtcccatctctctggtcctcttcacagcaaaacttccctaaagtgttgtctatactcattgtctacagtttcttttcattgtctattttttctctcatcattcaaaatattccacatttaaatttctctcactctctcctctattacgtcaaatgttcctatgatgtaggtatcatattgccccttttaatatgaggattttgaagcaaagagcttccagcaacatggtaacctgagctaaggaagactctcttccccctgcaaatacatagaaatgctaaataaaataaaataatatatcatggcttaaggtaagacggggaaatctccagttgccagaaactgggaagaaacatgaagatacagcagtgaatgcatgggtccacaaatccccagcactgcccagggctatccactgggcagagctccatgccccaaggccctctcaggcccctcattctctcttaaattcacattaatcaggatttcacaacattgcctcttagatgtctaatgggcatctcaaaatgaacgctcccaaaactgaatcctcgtcttccccacaaaaactattctccccacagtcttccccatgattaatttatttactgcaattcctttcttccagttggtccgagtcaagaagcttggcatcctctttgtttctgacaccctcaaagtgtcaccaaatccttttgtcacaatcagcctttttccaaaacgtcttttactaacaccctagtttccagcacagttgtctctggcctattacattgcagtcacctcctaactaagtgttctccctgttcctgctctagccctattcctgtgtattctcacagcagctagactaggtctaataaaacatttaaaaaaatagcccttttctgttcatgaaactcctattgcatccccatcttagagtaaaattgccagagtttttcccgtgatcctaaggtcctgccagtctgtcacagccacctcactgacctcatatcttatcatgctcctcctcctccactgtgctccatctgtacctgcctcttgctgtttctcaaatacaccaaccagatgcctgttctgggtggaaataactggtcatgactgataatggtttaataccactctccttgggatatttgcatttggcaagcatttggctaaaaggaagaacttccaataatggaatattcagtcatttcagacagctttaagggaaactgttgggaggaaataacctctagtatttctcaaatcacatagttagattatgatatgatatgacagacattaatatagtcataattccaaatgttttaatggtttcattggactttagaagtagtaaactttcttagttctccttttcccattattccgtagcaatcgtagaaaataacttctgcttttcctttttatcttgtgcaaccagcacggtctttgggtggagttttttaagtgcagaaggtttcagggacaaatatttacctctatatgtaaccagtactagaatagtcagtacctagaagccactcttctttgaaaaggattatcacctgatcaggttctctctgcatttgcccctttagattgtgaaatgtggctcaaggtcttcacaactttcctttcctttgcaacaggtgcttgctcggggctgaaggtgacagtgccatcacacactgtccatggcgtcagaggtcaggccctctacctacccgtccactatggcttccacactccagcatcagacatccagatcatatggctatttgagagaccccacacaatgcccaaatacttactgggctctgtgaataagtctgtggttcctgacttggaataccaacacaagttcaccatgatgccacccaatgcatctctgcttatcaacccactgcagttccctgatgaaggcaattacatcgtgaaggtcaacattcagggaaatggaactctatctgccagtcagaagatacaagtcacggttgatggtgagtcccctgtgagtgtacgagggcagcagtctagcatggtagttagcaaaggctcaggagactaaactacttcggctttagatcttggcttcacaactttgcacaaatcaattaatcctcaaagcctcaatatctcatggcactaatgacattatctacctcacaaggctttctgaagattaaataagacatgcacataagagatatacctagtgcaataggtaatcaagcactaagtataacttattgtttgtttgcctgggaactatcaagtataatgttgctaatttgatgagatggagaaaatcttattttctcaacaatagactgtaccctgggaagagtcctatttttgtcatgaagatttttgtttcagtacactgtttggaagaggatgagacttgtgcaagaatggtacttaaggacaatggacatgcctggaaatgtcctctgaggaggagtggggtcagctaactttcagacacttattttccttattgaaataagtgacatatgtaaaatatactatgtccaagcccaacttctgatttttgatctctacacctgtttatttgacagtattccccattttaataaatacaaattccattctatcaattgctctagccaaaaccttgtagtcaccgttgacctcctctttttctcacatcccacatccagtccacgggaaagcctattggctttaccttgaaaatatagacaaaatccaaccacttaccacccccagtactgccccactcaataccacaatcagccgcggcctgaactgtcctaatagctttgccacctactctccctgcttctattcctgcctccttcagcttaaaatcctcccattgcttctatctcactcagagtaaaagtcaaattattaacaatctgcaatgccccacatacccagcgcctatctgaccccattttcctatctgcatccccctcactcactcggttctggccatgctggccttcatgcttttcctggaacgggccaaaacacactcctcctcagggcctttgtacttgctgtttgctctacctagacagcttttctccaagatactagcatgctcccctcgtttcctttaggtcttggctcaaatgtcactttatctgtgaagcctttactgaccaccctatataaaatgtaatctcacttctccaacacacacacacacacacacacacacacacacacacacagacacacatgctcagagaaaatagaaagagagagagagagagtcactctctatctccaattctgcttctctctttttcatagcttttattactacctgggcatattatagatttttatttgtttattctttctcttccctacctagaatatgaggtccctaggagcagggactttgtattatttgctgctttgtcctcagtgcttaaaacctttgctgaccactagtagattcagcaactatctgtggaataaatgagtggccacagaggagactggggaggcttcagaggagtaaagatgcagaaggtagaagcaaatgcctagagagcaactgtggggaaaaagaaaagagagatctaaagggaatttccatgaagatttcctatatggtgtcctgtctccctcctaatagaatcctttcttgtgaatcagtagtaaagttgcatctgcttaggaaaatgcaaccaaaaacttagtaaagcctgaatagggttttaggccccatgccaaaattcttctcagaagaaaaacttcttcatctgagttatactgttttaagcaaatgtatgatcacaataccagtgactattctaatgattggttcttctcatacaaaactgttgatgtgttacctatttatctcataccacttacagttccacacacaggtacatttgaaatataagctttttctgcatattaaatgtacacaatttatttttcttttaaaatcataatatcataaacataaagcctttcccagtcttttgttcttgccagggaagatgaaggattaaattaaatagatgagccagttgattacacccttcttgaaaagtgctataagcttcagaattactcgactcccccataagttacctttcgacttcctttctcttatctttcactttcttctatcttctctgggatggagaatttggtcagtttgtgccctaatgcacattttccagtgtagaaaaatatgagcacaagtcaaaataaatctacctgaggaagatgcattgaccctaattaaaagcttgaaaggaaattgaagtctaaacattggacacaatcatctctcacttatgtatcatcctattctctctgtgtcatgtagtcatacatagaaggagattattaaatacacctctctccaaaatgcagaagagaaacattgccagatgtgaagccctcttcattttgctactgactgctgtgtaactctgggcaagccactgtttttatcgattaagtcaagaacttcaatgattaaagaaaattgccagaaatggttgcggattcttatataattctaccatgcttatataatccatcaactttacagttaaaattcagagagcaaatatttactcatttttggaaatttgctttgacaaatattattaagagcctactgtgtggcagatgatgaaataagactattttcctatgacttgactttccaacaggaatttctatcggatttgtatttttttctctaatttgataaaagcccaagaatatattttatcctattttcagactgagtcagtgttgaaataaatgtaggttttggtttgttaaagtgcctaacataatgttacaacacaaatgcaaagcaattaaagtttataattgcttcatgacatacatagaaagaatagttttacatatgtgattcactaaaattatccctcagtttcccaaaagtatagtatatatgttagctgtatattttgcagatgatagaaatttgagttcaccacatccatcccaaccagctgctattgggacattaggacaaatcaatagaggggaataaccataaaaattttgcctgtgaaagtgtacctttatggtgttggtgaaagcacaatgaacaggaaataaggaagctgacattctcagccacctataaagtaaagggcttcattagaccatccttgaaaagtaagttctggaatttgattatctctgactataaatgatattctcatattttgtcattttgcattttccctggctttttattctcttctcttctttagttattacctgagctgtctacatttacagtgtaaaatgctggaataatttctccaggagttcatatttgaaagacctcatagggttttattatatttccatgaaaagaaaaaatcattcatatctctcttaatattgcagatcctgtcacaaagccagtggtgcagattcatcctccctctggggctgtggagtatgtggggaacatgaccctgacatgccatgtggaagggggcactcggctagcttaccaatggctaaaaaatgggagacctgtccacaccagctccacctactccttttctccccaaaacaatacccttcatattgctccagtaaccaaggaagacattgggaattacagctgcctggtgaggaaccctgtcagtgaaatggaaagtgatatcattatgcccatcatatattgtaagtttatttttttttaactcatgagttgttttttgttgttttctgagaatatctcccaggatagtcacagagaatataagtaccagatgtcatttggctttttaccattattgtagttcaatagttctaatcacacctcctaggcactgtcttgtatgaaatatgtctgctagcacaatcctatttcttaaagatcaagttgaaatactggccaggggccactcaggttgtatttcgtgattcagtctattgtgcgacctcatttccagcggaaagcaagattctcaatttcaagaacccagaacaatgcagaggctgcaatgaaaaaaactgaaagaatatcttgagaacttttttcctctccaaatattaccattcctgaatttaacatactttattcttctcttgtaaaatattttctttacatcaattctttcttctagctctttatctgtatgtcttcccatccctacctgcctgtctttaaactctagccataggtttaaatgaaaaagtcctggggaaatttacttaagcaattataggcaaatgcaaatatcatttttgacatatacttttgttttgctatacagttcatattaatctggcaaaatgaaaataacatctttctttcaacatgtgcaagtctatgcaccaatctcattattatcacagaatatgctgtgatacacagtgtggaaaatataaaaatgggagagaaaaccataatcaaacctgcaaatgttaaatattatgctttaggctcctaagccttctttccttcacgatgttatttgaaagaatgagaataccccaatttgagtaagttcacactttacagaatctatcttttgctgacagactgaactttcttctttacattattttaaacattacctatgaaaatctcatagagaaacacctatcacatgcaaagactaacgacagataatagaaagaagaacatttagacaatttacaaatgtggcttccatttacacattgtaaagatgaattatctacatggcttaagaaagattttgactcattaattggtaattgtgaacattgcattactaatttttgtcttcactatttttactttcaaatgcagaattcttcacttggtcaacatctcatcttattgatattcattcattcacttacttattcatttatgcactcattcatttatagattatttgattttaaaaaatgaaacatcagggtcttgtttgtgctaaacaattactacctaccatataacatgtacaaacacaacccagacccattaatttaataatttttcttgctatcactattttagccctgtttttgttctggttggttggttttgttgcttttgagtaacaaaaattaaataaaatatatactgtaatctgaagattggttgtatatcctatctgtggatgtggcatccaaatgtttacaatctttatcattccctgcaagctgggaatatgtgccttggttggtgttattcacacttctctttcttcatctggttgccttcttcttgcctcatgacatcatcatcctttccagaaaggtttacctgcctccacttatctcacctcctcagtgtcactaacaggttcattgtgctccttcagcatcctaggcactctcattccgggtcctcactgctttatattttccttgactcttgtgaacaccccaaacccaaaccagagttctgcattcttcatctttgtatccatagggcaatagctgacagagattaagaaatcaataaagtttcttgaacttaactaaaatggaacagattatctttcttcatatcccttttagattccagtgttacttgacgatgctgtaaaaaggctaaattctcattataaggtgacagcttcttcattatagtgctttggcacttttttttcctgacctttagcaaaatcaagtattctgttttctaaaggcactgtttctgtgaagggttttttcatgaaagtaggaactattttttattttggcagaaaaagtataataaaatctattaagattgactatatacttatgaggttgtgatgtcataatctggtattggccttggtggaatacaatgtcaagccaataactcagacaagtatctacttgcagttcatgatatgccttcaagctccagttgtaaataataacccagtaaccactgcattttgtgtgcaacatatggcaatgctgcagtggagagctctaagtgataaaaattaaaggggaaaaaaggtcattcacagtaaatacttaccatctgctcccaagcagggcggcaaacagcatcatgatgtaaatttaggcactgttcccacacagccataaattgttaaagagaacagatgatatgtgattttacaagtcatttagtaaacagccatccccaaggcaaagggttcatttctattgggagctctctgccaaaggcaagaaagtaaaccataatccaaagtcaaaatggaattatccttctgctttcatcttcagtatcagaggtcactctagttgaatcactccaaatatctccaattggattgcccattcactcgaataatgtactaggctgcccctccaagtagagggatggtcagcctggagttctaatgataaaggaaataacatcatagtagagtcagaagaaagtatattgtaagcaacatgtagagtgtgagatgtagttaaatttgtgtgcatgtgtgataaagagagagagaaaaagatgcattaaagtaagacttgaaaattatccactgaaatgtggatattggttctctgagcaataaaattacaaatgactttcttttcatctttgtgcttctctgaattttaggattttctgcaaaaaaaaaaccgtgcatttcttatataaacagataaactatgttactttttaagaaacatatcagaagcaaggaactgaaatgcttcaaagtgttaggttccaagagtcggaagaggtgttgaggtgggggcaaggaagtttgagggatgtgaggctgcttctgaagagccaaatatagcatataaataaaacagacagaagagaaaaaattatgccaatcatgaaacccaataaatggggaagtaacgaaatcaaaatacacatgattaaggaagataagataatcagattactttctttaaaggtatttttattttaaaccagaatgaacttattaaattaggtaattgaccttatttactagccttggctctaaatgactatgggcttcctctacaaattaaacctaccctctgaaattgaaattgaagaattactgcttgtgagaatactccattatgctgcagggtcctaaggcaactccaaatgaggagttccccaaaaagttttgagcaatgtcagcatgcttgaaaaggagtaacactcatttttatgtgtattaaattactagacagtttgtttttatagccccattctctataccctgaaaaccagggggaagacgaagacctaaattgagtggctgtattttcgtgaccacatttttcttctttatcaagtctgccttgctctactctcagaaagaccctcagactttagattttgtacatagactcaggtctcttgctctcccaaactacggtaaattcctcctgctggtttccttcggtgtgcaccataactgcccgctgcttggtgatctttccttccctaatgttctatcaatgctgacaacatgaagggtcccttctgaaccacagggcctgagataatagccatacatagtatgtggtcaacgtctcagatgtcttcactatgcctagactcataccatgtgcaaagtaatctcaatattgatttcggttgagtgagcaaaaacaaatttttgttcccattacactacttactgtttagtggaattcagaacaaaagagttccatttgacagcacactgttctaaaccttttatcacagtggtttcacctgagactgtgtcacagcctgaaatgagagaacaggcttcttatacagtaacaggaaaggggcagaaatttctccttattttaatcctgtaccttcagttcctgattactttaaattgaaagtactaacttctgtttgctttctgaaagcaggcttatctcatgcaggtttattaataaaaacccatatggttccttcacatttctccatatgtctttttctctagaaagaaaacatagattagtctgttttctagctgaagataatctattgttttgtatattcgttttctgttatagcttagaaaatttgtctcctctgtggcagattggtcagaaaatgctgacatgatagagaaccactctgggaatctgcaaacttgggattggtttgccaatatccaaaatggttttactaggttggtgcaaaagtaatcactgtttttgcctttacttttaatggcatggcaagaatcctggtgtttcaaaggggttagaaagaatcttggcaatcagtcctctaacctgtctttttatattaaaaggtgaggaaaatgagacccaaaaaattcaaatgacttctaaagaatgcctagctagtgattgaaaaaattagttaccctaatgtttagttactacattctatttttctatatgttgagagataaatagcttatacttttaaaaaaatcaggacctttaagattaggccattactgagcaaaccatccatacccatatccagtttgtaaattccctaagaatatgaatgtctcatacctctatctcccgtatactaaatatattatctcatacttagcatcatctctcaaataaatatttattacttgattggatgattacagaattaatattaatatataattagtaattgattgtagaactagtactacagttagacaacaacaaactattatgatatagaaagttataccacttatactttatttctttttagagaagtaatgtgaacgattaagtgcatatattttagaaacccagctctgctatttaatagctatatgaccatgggcaaataaattgtttaaatatctctttcctcatcttaaaattagaataataacatctacctcatggtggttgctctaaatattaaatgttgttatctgcataagcacttaaagtgccttctcatggcagatatccaataaatattggtaatgatcagctgaaaggtgaacataagcactaaaactcaaagatttttagactataagccaggatttagactgaatgagaaaacctatctaggagaagaattttttaaaattttccttattctctgaagaaagcaatgggaaaagtgtcttcacctactggcaacctaacaccatagaaatggtacaatgctacaggagtactgcaatgtggaaaaaaacaccaggggctaagtagttaatgataaagaagatgtaactttttttttcaaatggaatcctaatcttagctggggtgtgggtgggaggattttaaaaattctttaactgttaaaatgttgtaaaaataccaaacacccctatgtctatttgcacagtgtcccatgtgaaacaatgtcttttataaatatttacatggcctaccctagcactgagaagaaaaatagataatactgaaagtatggttgttaaagagaaaatagtggccataatctcttgtaaaaatattttcaagtacaatgactatttcagaaatattaatgtgtagaagctaaaaagttgatctaatagacatagagagtagaatagtagttactagagatggggaagagtggggtggtggggggtagccaaaagttggttaatagacacaaaaatagttaatggacagctagatatgaggaataagttcttgtgttctatagcactagagggtgactataattaacggcaatttatggtgtattttcaaacagcaacaatggtagactttgaatgtacccaacaaaaggaaataataaatgtttgaggtgatggagatactaattagactgatttaatcattacacactgtatacatgtatcaaaacatgtgatcaaaacatcacactgtacctcataaatacgtacgattatcatgtctcaattaaaaataataataaaataaatcagcatataaaaataagctagtcacacaaaaacagcaagattgaatttgtctacaagatggcagatttatcaatacattactcaaaaccttagcatcacaattatgatgacagtatttgtaaaattgtatatgaagtttcaggcttttactatttcagattagttcaaatcactatttcaaatcaaatctcaaataattttcactttcatcgatatcagaataaaacatatttaggtcctcatcatttattgggagtctaagcaaggacctgctcgtccaattctaagatgaaaattttcaaaaacagtaaaatgctactaagctctcccaaactcaaaacctatgttgtgattacttgtgttacttatctatttttttttatattgatgcagatggaccttatggacttcaagtgaattctgataaagggctaaaagtaggggaagtgtttactgttgaccttggagaggccatcctatttgattgttctgctgattctcatccccccaacacctactcctggattaggaggactgacaatactacatatatcattaagcatgggcctcgcttagaagttgcatctgagaaagtagcccagaagacaatggactatgtgtgctgtgcttacaacaacataaccggcaggcaagatgaaactcatttcacagttatcatcacttccgtaggtatgtgtgacatacaaggaagggatcctaataaaacatgaatgcatgctggccttgcacttcccctatcttatacctaggtagaaaagaaaatcattgtgggattattcatatattctaaaaatctctctttccaaagttaactaagccatggtttctatgactcaaagaaaaaaaattcaaaatgaaataggggagaaattcaattcaattgtgaagggctatgaagaaaaagggaattccacaaattacttctaaatgtgcactcttactttcctagttttatgaacaaaaatataagagaaacaggctgttcatttagagggtttatatctaccactgcattaggtcagatgaataagtctccatgagttgggaatctggttttcctacttctatttgattccccttctcactgtctttcatgggctccccttcctcttccaccatttaaacagctgaaatctatggaagttcacaaggggagtctcaacaccttggaaattacaaaattctgtaagtctgtgaaatttggctttctctagaaatcatctgattttcagaaatgtcatcaacccgcaaagattaagaattacggcctaaaatattgatgtccaccaaattctcatccttctacaccttctcttctctctctacagaatggttttattctcctgtatgattttctgtatgatttcattcatttccatgtcttcatgtaacactggtacatcccaaatctatatctttaaagctctaaaaccgtacacccagctgcctctggatgtctctgcttggaagtgccagaagcaccatacaggtctgatatatcaaaactaaaccatcttctttcccccaaaattgctattctgtctgtattgcctatttaagtgaatggccctactaactaccaacctaagacagaaagctaaatgtcatctcagactcattcctcttgcctattccctggtcccacattcagtcaccgagttgtgtccatttccttccttcctatttctcagatacattttcttctctccttcctccttcttttgcctttctttacttatttctttgtattctcttctccttctccctctccccttccctttccctccctactcctctttctccttttcctttttcttcttcctcttctatgactacttctactactgctaagtccttctcagactctcctttgcacacaagtcaaatgccacttgggcaaaatctgggagcaggtttggacaggacatggtggttaaggtacctatgactggtggaagaacaagtttataaaagctactactcattaatatcttttctattgatgccttctctttcttcacctttatagcactggtacctagaaaaagacccataatacagagaaactcaatatgttttgttgaataagtgatgcaacaatataatttcttgtaatccttaggctcagtttttcagtatgttgcccttgggccatctgcatcaagatcacctagggaggtcgctaaaaatgtagcttcattaagacacctcagacctattggatcaggatctttcaggtagcactggagaatctgaatattcagcacatacaagtgtgacaaccacttgtttagtatattttatctccagagtgttttgaatttactaaaaagttcctaaagagccatgaagaattataagactatcgcaaacctacaggttgataaatgtatagtgaatgggtttctcgtaggtactccttggtaccaggaggaaaaatcacctcggtattttgggatgtatatattgtcatggtgggggttttttcatatgaagaaaatgcactgttcattatgatagccactaacctcatttggcttttgagcacttaacacattgagcacttaacacaatgtgcttaacacaactgagaaactgaatttttaattttatttcatctttattaattttaatttgaatgtaaatagccacatgtttagtggttaccatgtcagacaatgcagctctagaatttttacctgagttgcttcatttgggcagtagataataatatatatttcttgtagacccaaaaagcatatgtgttttagggtgttataaattaagcacccattcattcattttttagcactcactatgggccaaatattggagtacgcactaagaataacatatgagaaaggtaattcctcctaataagaattcctgctcacaatctaacatgagtggcaaacacataaacagctgaggtacatcctttgctttccattttattaatttttataggcttaaccagattttcaccccagtagcaaaccaagacataattgtacagcaagccataattaagcacataaaatagtagcaatcctggtatcatcttcagcttgtttctcatgctcactgcaagtgaaaacatggctgtaatgggtagaagtatactacagtaaggctttcccactgcctaaactttctgcattaatatttagctaagatattctcataatatcagaagcccccaatatttgattaagatcttattaccaaggtctaaagacagctacagtcttaacgtcagataattgccttttgcaaaatctaaaaaacctgtttgacctgtacaaagaccaagcatttgttattatagtaaataagaagagcaggtgcttatttttcagagggattcagagctgtcaatattagtgacctctttgtggcttttagagctattttctaacccccaaatgtccacagagctttaactcaaacagcaacaagatggcaaagaaaatgaatagtattgatgtgttttgttaaatatttgttttttctaagtttcaaagttaatgaacctcagcaggacagattaagtatacatcaacctacagttataaatgaccatcacttgcaagtatcagttgagcctactctccatccactctgaggagtaaactcttgagatctgtggagaatttgaaatcatttaaaaggttaaaatacctgcttttgagatgtttccagaaccaaattggtgtttggtgtcgccagtgaagctgtaagtaagagattaacctcttacttcttaataaggcaaaacatttggctgaccaaaaagttttagcaataaaacgtggaagccaaaggaaaacagagccagagatgactccaagttcacagatatggaaagaaaatgacaaggtcaggaaataatcaagttttattggggattttcagtggggagaggaaggctggagagaacaattagttctgtgtttttctctttctaagactacagcaaatggtgactttcccctctttggactatgattcacatctaatttgagatattaattgacctttttgtatctccatggaaatatcctaaagaaaacagagacaacaggcaagttcacacagctagtaagtgataaataatatctgaattcaaagcctttttttttttttttgagatgaagtttcactcttgttgctcaggccaaagtgcagtggcacaatctcggctcactgcaaactctgcctcctgggttcaagtgatcctcttgcctcagtcccccaagtagctgggattaaaggcacgtgccaccatgcctggctaatttttgtatttttagtagagatgaggtttcaccatgttggccaggcttgtctcaaactcctgatctcagttgatctgcccacctcggcctcccaaagtgcagggattacaggcgtgagccaccacacctgacccaaagcccttgtttttaaccactatacaaactgtctcctttgatataactgagaatgttggtgaaaccatatggtctttctaaaaggtaggtatagggtcactttctcatcacctatccaatttaatgctatgtacgtgtactttctcatcacctatctaatttaatgctatgtacgtagagatactctggtttgtctacaatgtgtataatttcatattaatgcaaagttaggaaatacttgtaactgaaaataatgtctatgataacatttcaagaaaagaatagctattaaaataataaattagctttgtgttttgcaaaacaagattttattaccttaattacggacttccttgaatgcagatgtccctactttaaagttttaactgagccaaaattgctattgtgacatgcaatgtatagacagacagacagagagatgatagatagatagatagatagatagatagatagatagatagatagatagagaatgtttatggaaatgtaatcatctgatcagtattcaaatgttcacatttaatgaaggcattcaattgtataaatagaattccctaaggccatgtgggcatagtttgagatttatccataatagaaatgttttcaatttgtacgtgatcctgagaaagctaccattttttacatttatttatatttcccaacttttcttaaagaagagaacaggaaaaagactcaaggcaggaaaatgaatttactaattttttctatctggaaatttctcaagtcaagatgtctaggaaatgtatttcactttgggtctttggaaatgacaattaagatagaactacaaatctgacattcagaccctgagacctaaggtgatgtctccatgagcatccagcactgaacaactgtgtctgtcagtgggcctatgatggagtgtggaaatggcacactcctcaacgtggcaccttcatctccggagggacatgagtcagctgccataatcagtgaacagattggatgagagagatctcaagacttcattttgatgtagaaatggatacacagactggtagcatatggctgtcctgccactgagtgcagtctcaaacagcagggttgcgataccatggataccacacctcttgaccatatttgcacttaacatgttattccattttatttgtttgcctaaggaggaggcaaatagcatttgcactatggctttggcacatgagtccatcatgtttaaataatggggagaatggcatgctatgtgctaaagaatagtataaactctctgaaaggagaaagaaggaagctcctgcaggtgtccagaactgcagaagtgatccctatacattctctatggcaatagggtcagaatttcctgctgtcttattttcaagttcaggttctggaagttcatctttgcaaaaacacttgcctgcagctgagaccacaacaactgtgccccttgatttgatcctttacatatttggcactttttgtgtgtgtgaaaggaacaaatttcaaatgctggacctagtaaatcttgtgtgttctgggatagaggagcagtgttgagagcagagagccagaatgctctagagaagaggactaacaggaaagatcatttcttccgagaaaaggattgtccctattcctcattctccattgggaagactcttggcctttgccacagccgtggttttgggctgacaggcactggtcactacaggaaagctctctgatgtagggcaaacacaggccaagggaagaggcaagagggcaataaggaaggagcctaaagaaataaggaagtaatggaggaaggaaacagtgggatcctctgtaggagagaatgtgtgcaaattaggtcattttagtaaatacatatcaaaagggacaaaaagttaaaaatgaaggagaagggaacatatgtatcttatctagtctaagacatccagtcattgtatattaagcctactgcctgactggagaatcacagaagtgtctggcagagagctgatgccggccagagtgaagcttcactattagaacgggaactgcttggaaatagtaattattagcatatgttcaagtttaagagggaaaaaacagccgagactactcaagtgtttttcagcagttttgctgatggtaaagcacattgcaaaaacttgaatagaataaattagcattgaattcttttttgaggagtcttcaggggttatagaattttggtgatggcagtctggctattaagaagtgaaagatttgaagcacatgaagtcgtatgtcagaaaaagttagaggaaaaactgatatgtaagagcctacctactaattggaaaaaccaatctttgaccacagattaacagaaaaaaagttgaacttgcagtcagaaaaaaacctgtctactctcaattctaggacttactgactatggcaaatattttagtttctctgagactctttctttatctgtaactgtaaatggtatgaattttcttttgtaaatggaaaacaaattgtacatttgattttatgtatatataatatttaatatatatttaattgtgcattatctgaaaattcccttagctagtttttattttattgtcagatctaaaatgacgaaatagagaattcacttgctaaaagtggtgcccaaatcatacgatatatgttaaatcttgccactctattatttggtattttttttcaattctttcttttttattatttatttcttaccagtttttctggtttttgtggtttttttttttttttttggtttttttttttgcttttttatcactgtcactgtctcagagagcattctatttgcctttatcttattgacaaccgcagtgaagtatgggtccttctaaatttatgtcttagaaatgaaattaaattttgttgggcttccaaaagtgcacctctaggatggtagagaatctggaatccagttatgtaaagaacaacaaaatgatcttgggatgctgaatctgaagaagaggattctcagggggagatgtgagtttcaccaaacaattaagatgacatcatatacagagaggatttgatttatttgtcattgcttcaattggtagaactgagtagatttgggcttcatttgggagaaggactttgtttcttctataatgattaggtaagagtggtggaaagagcatggaattgttgttagacattgctaaattcaaatttgagtcctggagtcttagatttcccaactgtaatatgaaactaattattgtatcatagaagcagtaagtggtataactattgattagaatttttaatccctaaagtctaaaagacaaataaaaatgtataactaacaaggaaatagagaagaaaagataaataaaattctgattaatctaaattaggacaagaaaggagagagcaaaggaataaaaaggtaggaaatatagaaaaaataataagattgcagatctaaactgaaatatattactaatcatactaggtcttagtgcgctaaatattccaagtaaaaaacaaatgtcaaactggatacacaccttatgtatgaagatactgaaaggttttaattaaatagatgaacagataaaccatacaaaaaagaaagctagcatagctatatagcaggtcttctaataatgttatttcatttcaacatcatttcattataatattgatgagaaaaaatatcaattcccactggggccactgtgtgtgtgcagtttgcactttctccccctacctacatgggttttctccaggtattccagcttcctcccacatcccaaagatgtgcacattcagtgaactgtcatatctgcatagtcactgtctgagttagtgtgggtatgtgtgtgagtgtgccctgaaatgggatggtaccctgtccagggctagttccggccttgtgtcctgggcttccaggataggattctagccacctgcaagcctgaactagaataagcaggttggagaatgaatgaatgaatacgaatgattgtcaaataaaaatttataaagactaggataatcatataaatgcatgacaataaatgatgtgatatgaaagtgctccgcaagactaccagatttgtgatgttagtttttgaactgcatgatgctaggaggtgctttttacaactttcactttgtaaacatttattccttgatttaacccattaccactaccactgccatcattcacagattcaccaaaaattgagtaaataacgatcttacttgttttattaatctttattcaatgtatgtattcattcaatttatttcagtgtttgatattagaagtgtttgggatctttatgtagaactctgatatttttctgaccagaaatatgttgtagagacttagctcttgtttatatcaattagccaatggttaaattgatttcattatttgttgttttgctcagtcacagtttccaagaatcagctgatgttaagtgaggacttaccgtactggtataagataatgtacataaaaattagatgaattagataaattcttcaagacaagaagcattaggaagtgcacctaataaatagcaaaaattgaccgaaaaaaatcacaaagtgggagattttaacatatttatttcagtaactaataaaacagacaaaaactcagtaggaatagagaaaacctgcctcacctagttgaaataatataaatactacatacaacaacagcagaatacacccttttcatatgactatttgtatagtcattaaaaaaaatcaacaaacttcaaaggattgccctctaacaagaatgaaattaaaccaaaaatcaataatagaaacaccactagaaaactcccaaacttaagaaaattaagcaacagacttctaaacccataggtcaaataagaaattgcagtgaaaattagaaaacattttggacttaatgataatgaaattactatgttttaaaactcagaaaaatgttgctctataattacattgcctacacttgaggaaatgagggaaaaaaatagtggaaattaaacttaaaagtgtattgtgtgtgtttataaggtgtgttaacatatatatgtgcaattttttgaagactttgttactagcatgccacagatatagggtcctatttacataacttcatgtaaacttctctggctcccccaagtcatagagtatcagagcaagaagagaacttaaatgccagtcactctttcactcttgcctcctcattatactaatgtgaaaatcaaaaccctgagagtcaaggggctcacccaaagttacacagctctttagagagaacaatcccacaagagaataatatctgagaaaaggctaagatctcacaaagagactacccaagtgcaaagtggttccaaactaaaaggtcagccttctaacagtgggagaatataaattattctacaactttttatgaggcaaaaataatctaaaaagcttcatgatgttctagtctatatctaggatcaataaattgccattctagttcatttgaagctgccttgcatttatttcttctagccaaggtttgaaagagtttaatttaaaatctgtttaaaggcaataagcaggacacagatattataatcccatagaataacattttaagaatgcagttctctgaacgcatgtcagaaatttataaactccccatcagaaatggtgaagatttttgataatgaaaaacaaccttgtcgaactctttaggacttaagggaaatatgtgaatggtgctgtattgggaaattagaagagataaccgatttccatgatgtgtttactcgatttttgcacatatgatctacactaccttattaaaagattcctgtaagattaagaaatggaagtattctgagtggaaaagccaagagagttaatgtaggttcatactgagagaaagaaaagattaaggcttcctcccttattttggctgttggagatctcagcagagattttatgatgaaagatgacacagtgcaggctggatatttattgcagagctgtgtagccaccatggtctttaccacacccagattttccaggctgttcaccattgctgaatggaaatggcatcacatgactttcactcacacattaatctggacagagacaagcaactgccatgatttatttccacacccatatctgattcacatggtaatatggaagacatctgttactttcatttgttggaggttaaaactacttcctattatatagattgactatataactaaacatattataacctgaagctggtgttaacatagccacagttaataagttgtgttaaaattatattaaagggaggcatcattaagtgttaagagtctggggtaacatctggattctaccacttataacccaagtgaccttgggcaagactcagcttcctcatctattggaagggatgataaaataatctctccctggtaggattgttgtaaagattcagtgagttaatatgtatcatgcacccttagaacagagcagatgttattagcaccgtattttatatagtgatattaataaagggaatgactgataaaacaaagtgaattctagtcaaaaggtgggactttaaatatttttttgaaaaatcacacatacatcctattttaagaattgacatttcttttttctttttaatttttttcatcatttatggtaagagacatttctttattggccaaaaattactaaagtgtgtttgaaatgaacacctcccctcttgcacctttaatcacataccatctccctagaaaattcacatacttgaattatgctaaaaaagcaaaatggaaggaaaatgagagaacaacttagcatttaggaatgtagagacaatgagatgaaacagatctaaggtggcaaacatcactgagaagtacgacatgagatattggctacacctagtacaacacagtcagttttaacttgggcagtgcaccacgtttccaatcacaggtatccctagggagtttagtgtgtgttctaaaataagtgcattccttccatttggtctctctctctctctgtgtgtctctaatatacgtgtttatatatttttttcaatagcgtctcatttaaaatacggcatttaaaaagtcaagttatatactgtagcaatttattgctaaaaatatgtttatctttttatttaggactggagaagcttgcacagaaaggaaaatcattgtcacctttagcaagtataactggaatatcactatttttgattatatccatgtgtcttctcttcctatggaaaaaatatcaaccctacaaaggtaggttaaaattacaaaaaaaaaattgaagctgtatatatgtgtactaaaaaaaaccttaccatttttcttctctcttgattttagttataaaacagaaactagaaggcaggtaatgcatctgtttctatgaaaatagttgttttccttagatgcttattcctcaattattatccctgacaaaaatacctattttgtcttttcaggccagaaacagaatacaggaaagctcaaacattttcaggtaatggccaagctgaaaagggctattatcagttttaatgtatgttaattagttgtgttaatattattatatattattaatgtaatataattaatatgagctaaaattaatataacagtctcttacctttgagttagtgccttcactttacaaagtgctgtcttctgtattggttttatataagtcacacaagtctgtaatatcaggcctggaagcactcttaactccattttacagtcaagagaactaataatcaaggagattaagtcacttgtccacagtcagtaggatcaaagaaattagttctgctaactcattgatcctattattattttgaaaatgccaaagggttctatcactttgttcattcctaaggtaggtttcatgctggcagctccatcccaaagagaagaggaaaagaagatttggcaggggccaggattgcttctttgtccccactgtaaccctcattatcatcaatattgactgtactgacttattataagttaacaaagttttggctcaggccacaaaatcacagggagccaagtttcagttttatttcccctcatgtcaccttaggaaaattatttttcttaaccgcaacttcctcttctatataatagggataaaaacttcccccttaaggttgctatgagaattaaataaaatgatataggtggagtgcctgatatgatgcctggatgtgtaataaatgtgggccttcttcctcttttctagcccttcctgacttataagtgacagaatgtcagagctggcaagggccccagaggctatctggttcctcctaaccacctcccccgccatcattctatagatgatagtttatgtggattgatagtgtaatgtattttactttttatgtatgggaagcttcagttagctatttactctttttgttctgatgatctgttacatctgttagatctgttagatctaacagggtgcagtagatattacgggtggatcaagaactggtgtgaccttcaggcttaaaccctggtgaccatgagtctctggtaactgcaagttacaaaggccaaggttgcccacctgtgtgtatttaaagatgtaacaggctattctcacgtctggctttaagaataattgttccaattcagtttcgattctcagcttcagaaagagtgatttccaacactgcttaatggcaatacgactataggaattaggaaatgttaaattcattttgtacctataccatttaaaacagacaataaagataagttttacaaaggtttaaaaaacaacagcaaatcaaggcattcggtttgtaagcagtatttgttttacatttcaggccatgaagatgctctggatgacttcggaatatatgaatttgttgcttttccagatgtttctggtgtttccagggttggttttcctagtggctgattaaccgagaagtagaattctgcttcaccagaggtgtaaaaagcatttgtttaagcactgggcagtgtggtcaaatggctgcattacccattactgcattaagcagtgttgacttactccgtaatgaatggcattgccagatttgggaagctaaagcatcttacttcgcctttataagtaaagctaacacaaataaggaagttatgctaattcataaatagtctttacctggtaatctcagtgaagagagaactaactaggttgaaagtcaagagaactgaataaacaagctgggcgtggtggctcacatctgtattcctagggctttgggaggctgagatggaaagatcacttgagcccaggagtttgagatcagcctgggcaaagaccccatcccaacaaatatttaaaaattagccaagcatggttatgcatgcctctggtcccagcttcttgggagaccgaggcaggaggatcattgagcccaggagttcaaggctacagtgagccgtgatcacactaccgcactccagcctgggcagcagagtaagaccctgtctcaaataaataaatactgaataaacagcggatcaggagaccctaatttaagtcattctgcatcacttgggtgactgcaacccatcactttactaaactctctctgttcctcagttttccctttattgccccctcatatcttttgacaacattgcaaagacaattgatcacacccttgaagatacctcatgcttcagaaggaagttaccatataggcccaaagtatgcaagtatacatctttattattgccctaaaggtgcatgagatggcaaaggagctttcatatagttgaagtcatttcagtccggaaaaaaaaaaaaaaaaaagatcttttaaagtaatcagtttttcttaacagacatgcagaatgtttctgtgagcaaatcagattattaacatttttatagatttgtatagaaccaagttttcctctgcttccttattctgctatgagagtccatttgaggaagggcaccataaaaacagattacttgcttccaggcattctcatttccatttgttaaaaagactatttgccattagggaatcgtgacttctcttgttcagaggattctatcagtgtataaggttcatctagtaagcttcctcgccacagagtttatttgagaaatgtcatgtgagtaatggagagggactgattggatttcatcctccctaagggtgagtcagtaacagtcacagatgaagcaaagaactactgtgcaaaggctttgcaataccctattcaatctcaggcaggattggctcaccttcacttccacaaccaaaagacaaaaacaaatatttgatggatcagttatttcaaacagcttctcacgatggtagaaagcaccactctgaggtgtggcttcaaattggagagcaggaaactgaagagctaagacttataattgaatttttgaataaggagtttcagtttttttatttaaaataatgcaaacatttattcctaggaattcactggaaaggagtgagatttatgtaagaaattttaaatcttaccatgtgattaatcactactcaattttggagttttactgggaggacctcaattggctcaaaatattatagccctgaagaacttaagcagagaaataggggctttaattacatagtagaagccctgcttatttctatcagaaacagaataacttgatttggagccatgtccatcttcaaagctgcaagctcctgaggaatctgctgaggttcttaattggccattgcaggtgctttggggaagaagtgagaaatcaagaccaacaaccaaaggaggaggatgaggacagggacatgaggaggaggatctgcatgagaagccagaatggcagctatgtatacgtagctatgtaatggaagattagcagccaaatgcaatggcctttagaaaagaaggaaaggtgataataaaaccaagatctcaaaactgaacttgagaagggaaataaattaaattattaactttctgagtttttaaaaatcaaaataagctaggtgccacggtgtgcacctgtagtcccagctgttcaggaagctgagttaggaggatcacttgaacccaggagttcaagaccaacctgagtaacatagcgagaccctgactcaaaaaaagataatcaaaataatctcttggcatattgtgtggtatgtacacaaatggattacttctgtctctaccaattgcacatttcattgtccatttagcacctctttcaggagttagacagaaaacaattgcattaagtgagcaagtttattatttaacagctctaatattaggttaggtaagttagtgatcatcaatagattgcttttaccgtagctcctgaataccttcatacaacaaatatcaagagctgatctcaaggatagatcgtaaaccaaaatttccttgacacaaaaatgacagaagaaatcacatatactaaggcttcatgtgagagctatagcaagtggaatggaaattagtggtttctagaaaaacaaaaagctaagatgttccactaaaacctagcaggtcaaattaacatgagatagtgaactcttcagagttatcaagagcaaagagccctcgaggtgaaggtgtataaatatgaaggactgaaagtggacagaagtaggattaaaataggaagcatttgggatggggaggaaaactagacttcggctaaataattgggagggagtgtttataggaagaaggaagttatgaaatataaggaatgggaattaataaaacacaaataccactttcaataaaaatacctgttggtttttaccagccataaatacactatcctttccacattcacctaagcatttagtttttacagtccattactacatttaaaccaaaacattttcctttgagtgttccacattccttggtactgtctttacttatttgcaatatgatgagggatttgggctgtatattgttcttttcttttaagccacccttgcgcacataattggcaaatagtgtcaaaagggtaactgaggtatagaaaagataatgatatatgctatctgattctaagatatcctgatatcttctcttttgctattttttctttctataatatttcctttacagtactgccagctttcttcttaaatgaagaaaaaatccatagctttgtcaagctatccttcttctgaccttatagctatgctgctgcacaaactgccatagggtaagtgcagaaaagtgtagcaaggaggaagtcaactttctagggaagataaatagttctaattctctaatcatcactactgccagttgttttcaactatgcaacatagtggaaaaactatttactagtcttgagataatttatttttaatgtatcacattttctagatcccaagcaggtctgttccagcctctgattgtgtatcggggcaagatttgcacagtacagtgtatgaagttattcagcacatccctgcccagcagcaagaccatccagagtaagtacgaatctgcatttgatggtgctatggagaggctaagcatttagcttttgctttccccagaactgattaaaaggcacagaagaaaaataccaaaacaagaaattatagcttctgtacatttgaacacacttccccacctgagcataattttgagaaacaaaacaggcaaacacacaaatatcttcgagggccatgcacttgggccagtggactagatacaaaactctagaatcaactttctcttgtgtacattagtttctcacaatatttccccaaggaatccacatctgggtatgaaaggaatgttcaggctaattctgtagccatcttaatgattacttagtgattttccagtggctatggaagttttatggatgatttgtcactaaaaataggaccagtaaaataaaagcctacagagggtccacagcacagtcagcaccaatgccaatcctcttattcaaagtagcctattcccaattgagtggaaccccattgaggactggcctctcacccatagaggacaaaacctgtactgaggacaaagctgggaacctatttatccagttaagctcaaggctgtcactaagaagtctccctttcctccatgccgtggagaaataaagcctacaatcttttgaaataataaaatgaacttctcttttttgtgggctatagctgcctgcagtcataactgccacagatcatcttccatcatttctttgtcacttattgactttgaaaagaacatcaataaccacatacagaaacttgccctctggcaaaataaacagtttcagcaaagtaattcatgcacacttctgggtaaataccaaaaataacagctaggaatgaacaaatccaggaaaaatcatcttatggtgaggaaacctagtgctggggggactgtgcgtcatttaaggactaaataagtggcaagatacttcagagaacctcatcagagaaaatttacctagagtcacataaataaggaacagactgttggagttaggaacttctagtttgaaaaaaaaatgttgtaaagcattgcattatcaatctagtagaacacaagagtaggatattattacatgatattttaagaataaaaactctaaaatgatacctaattctctttactgtctataatagaaaactaggggaagtttgtttgctgttaaatataaggcattgaaagaattttgagattatgtctcctttctaagatttaatctaattaaaatttctatcctatttaagttcatttgtttttctatgtatcctttcttctaactatcaatttttcacattagaatgtaaacaaaccaaatgacaacttggggtttatcttttaaggttattaagaacgaaataaattttaagtcaaaattgtgaatatggcagctacaatttaagaacacaaaataaactgattgggttgttttgcgtgtcaaaaaactgattcttaaaaaaaaaacaccctttgaaactagatgacttttagacactagcaacatcttttgactttcttaagaatataaaagtttatttcataaataagatttgagaattgaaaacaaagcagaaatgttctttttcaggcattcaaaactgctatttaaaaagtattcaaaaccgctattgggacatggtcctccaaaaggcagtcatcttcaaaaccccagctcatgctaccagcccaaactgagtgacatctccacctcccacagcctctctcttctcaccttcttcttctgtcttctcaatcttttttgtgtttgtcagttcatctatgtagatctttgtcttccagtggctctctcttcttttaccttcatcctttgaatccctcattttctgagtctacccttctcttttcctctccttctccataactgtctcccttatccgtaatatccttcttaaaattttcctcctctgtctgtcttccagcattatctaaacggggagccaatattttctaaagtgttattttatttgaattcagatttatgttactaattctaaatttttttaataaataagtgaaatgcttcctttttctttatcagaaactattttgcacttattttacttataatcacttcacaaacatttccaggacatttactatgcgctagagcaagacgttaaataagactcgatcttttaaagatttcctagtatagtgggctgctgtttacttattttaactaaagattagtgattcttagtaaaataaatatttctcagtagaataaatgccacctatagatgcagagagcatttttctttcctgattatctgtggttttattctcctctaggtccaattaaattcaaagatcaacttcatgagtaatgcattattataggtggttggtgactctctcccatctattcccaacttgaatgtgccagctctggggaaagaatcctaacttctctgctttcttttatttggcagtcacttctgagtaggaaagacagccaaacaattttattctttggctcttagactgttgtaaaataacagtgttattttctttacttcatgtgtctattacctaattggactgctgtttaacaaactggtaaattggtttgtgttcatattctagtactcagcccaattatttatttagtgaacttatggctggatgtatttcaatgttgtctattgacctcattaaatgctcttggtagggtttatattgaacccaagactgacgttagtatattgaaaacaggactttcaagttaagtaaacctttagtttcgaaagtaaattatctacaaaatttaataattgctgactcttatatttatttgttgataaatgtaattttcttgagctagctccaaacattcttctaatctgtagccctggaaagcaataaaagaatttaaagttcttttacctgcagacctgaaaaatctatagaactgtttacatattttgtatatctcttcactaggtgaactttcatgggctaaacagtacattcgagtgaaattctgaagaaacattttaaggaaaaacagtggaaaagtatattaatctggaatcagtgaagaaaccaagaccaacacctcttactcattattcctttacatgcagaatagaggcatttatgcaaattgaactgcaggtttttcagcatatacacaatgtcttgtgcaacagaaaaacatgttggggaaatattcctcagtggagagtcgttctcatgctgacggggagaacgaaagtgacaggggtttcctcataagttttgtatgaaatatctctacaaacctcaattagttctactctacactttcactatcatcaacactgagactatcctgtctcacctacaaatgtggaaactttacattgttcgatttttcagcagactttgttttattaaatttttattagtgttaagaatgctaaatttatgtttcaattttatttccaaatttctatcttgttatttgtacaacaaagtaataaggatggttgtcacaaaaacaaaactatgccttctcttttttttcaatcaccagtagtatttttgagaagacttgtgaacacttaaggaaatgactattaaagtcttatttttatttttttcaaggaaagatggattcaaataaattattctgtttttgcttttatgtggtcttaattctttgtcttgtagagtaaaaatattacataaaaatcaga

SEQ ID NO: 8 - RNA, 2145 nt, Homo sapiens
  source 1..2145: mol_type = genomic RNA
actgagaacagggcactgcatgcatgggacaggatgctttcatggagcccttcggtgacacacttggggtctttcagtgcaaaatatacctccttctcttcggtgcttgctcggggctgaaggtgacagtgccatcacacactgtccatggcgtcagaggtcaggccctctacctacccgtccactatggcttccacactccagcatcagacatccagatcatatggctatttgagagaccccacacaatgcccaaatacttactgggctctgtgaataagtctgtggttcctgacttggaataccaacacaagttcaccatgatgccacccaatgcatctctgcttatcaacccactgcagttccctgatgaaggcaattacatcgtgaaggtcaacattcagggaaatggaactctatctgccagtcagaagatacaagtcacggttgatgatcctgtcacaaagccagtggtgcagattcatcctccctctggggctgtggagtatgtggggaacatgaccctgacatgccatgtggaagggggcactcggctagcttaccaatggctaaaaaatgggagacctgtccacaccagctccacctactccttttctccccaaaacaatacccttcatattgctccagtaaccaaggaagacattgggaattacagctgcctggtgaggaaccctgtcagtgaaatggaaagtgatatcattatgcccatcatatattatggaccttatggacttcaagtgaattctgataaagggctaaaagtaggggaagtgtttactgttgaccttggagaggccatcctatttgattgttctgctgattctcatccccccaacacctactcctggattaggaggactgacaatactacatatatcattaagcatgggcctcgcttagaagttgcatctgagaaagtagcccagaagacaatggactatgtgtgctgtgcttacaacaacataaccggcaggcaagatgaaactcatttcacagttatcatcacttccgtaggactggagaagcttgcacagaaaggaaaatcattgtcacctttagcaagtataactggaatatcactatttttgattatatccatgtgtcttctcttcctatggaaaaaatatcaaccctacaaagttataaaacagaaactagaaggcaggccagaaacagaatacaggaaagctcaaacattttcaggccatgaagatgctctggatgacttcggaatatatgaatttgttgcttttccagatgtttctggtgtttccaggatcccaagcaggtctgttccagcctctgattgtgtatcggggcaagatttgcacagtacagtgtatgaagttattcagcacatccctgcccagcagcaagaccatccagagtgaactttcatgggctaaacagtacattcgagtgaaattctgaagaaacattttaaggaaaaacagtggaaaagtatattaatctggaatcagtgaagaaaccaagaccaacacctcttactcattattcctttacatgcagaatagaggcatttatgcaaattgaactgcaggtttttcagcatatacacaatgtcttgtgcaacagaaaaacatgttggggaaatattcctcagtggagagtcgttctcatgctgacggggagaacgaaagtgacaggggtttcctcataagttttgtatgaaatatctctacaaacctcaattagttctactctacactttcactatcatcaacactgagactatcctgtctcacctacaaatgtggaaactttacattgttcgatttttcagcagactttgttttattaaatttttattagtgttaagaatgctaaatttatgtttcaattttatttccaaatttctatcttgttatttgtacaacaaagtaataaggatggttgtcacaaaaacaaaactatgccttctcttttttttcaatcaccagtagtatttttgagaagacttgtgaacacttaaggaaatgactattaaagtcttatttttatttttttcaaggaaagatggattcaaataaattattctgtttttgcttttatgtggtcttaattctttgtcttgtagagtaaaaatattacataaaaatcaga

SEQ ID NO: 9 - protein, 462 aa, Homo sapiens
MGQDAFMEPFGDTLGVFQCKIYLLLFGACSGLKVTVPSHTVHGVRGQALYLPVHYGFHTPASDIQIIWLFERPHTMPKYLLGSVNKSVVPDLEYQHKFTMMPPNASLLINPLQFPDEGNYIVKVNIQGNGTLSASQKIQVTVDDPVTKPVVQIHPPSGAVEYVGNMTLTCHVEGGTRLAYQWLKNGRPVHTSSTYSFSPQNNTLHIAPVTKEDIGNYSCLVRNPVSEMESDIIMPIIYYGPYGLQVNSDKGLKVGEVFTVDLGEAILFDCSADSHPPNTYSWIRRTDNTTYIIKHGPRLEVASEKVAQKTMDYVCCAYNNITGRQDETHFTVIITSVGLEKLAQKGKSLSPLASITGISLFLIISMCLLFLWKKYQPYKVIKQKLEGRPETEYRKAQTFSGHEDALDDFGIYEFVAFPDVSGVSRIPSRSVPASDCVSGQDLHSTVYEVIQHIPAQQQDHPE